(19) United States
(12) Patent Application Publication
(10) Pub. No.: US 2023/0114408 A1
(43) Pub. Date: Apr. 13, 2023

(54) METHODS OF MODULATING T-CELL ACTIVATION USING ESTROGEN RECEPTOR BETA (ERB) AGONISTS

(71) Applicant: Ohio State Innovation Foundation, Columbus, OH (US)

(72) Inventors: Shyam S. BANSAL, Lewis Center, OH (US); Richard Joseph GUMINA, Columbus, OH (US)

(21) Appl. No.: 17/910,158

(22) PCT Filed: Mar. 11, 2021
(86) PCT No.: PCT/US2021/021903

§ 371 Date: Sep. 8, 2022

Related U.S. Application Data
(60) Provisional application No. 62/988,251, filed on Mar. 11, 2020.

(51) Int. Cl. A61K 31/05; A61P 9/04

(52) U.S. Cl. CPC A61K 31/05; A61P 9/04

(57) ABSTRACT

Disclosed are method of modulating immune response in a subject using ERβ agonists. The ERβ agonists can selectively inhibit the activation and/or proliferation of T-cells, reducing circulating T-cell Levels in a subject without significantly affecting circulating levels of neutrophils, monocytes, or B-cells. As a result, the ERβ agonists can be used in therapeutic and/or prophylactic applications, including to treat or prevent chronic heart failure (CHF) in a subject post-myocardial infarction (MI) and to treat or prevent graft-versus-host disease (GVHD), multiple sclerosis (MS), and/or experimental autoimmune encephalomyelitis (EAE) in a subject.

### CROSS-REFERENCE TO RELATED APPLICATIONS

This application claims the benefit of priority to U.S. Provisional Application No. 62/988,251, filed Mar. 11, 2020, which is hereby incorporated herein by reference in its entirety.

### STATEMENT OF GOVERNMENT SUPPORT

This invention was made with government support under R00 HL132123 awarded by the National Institutes of Health. The government has certain rights in the invention.

### BACKGROUND

Activation of innate and adaptive immune cells underlies inflammatory responses in many chronic diseases. Monocytes, macrophages, and dendritic cells (DCs) mediate innate immune responses, whereas CD3⁺CD4⁺ helper and CD3⁺CD8⁺ cytotoxic T-cells arbitrate adaptive immunity. While innate immune cells comprise the first line of defense against acute injury, chronic inflammation often implies activation and clonal expansion of specialized effector T-cells following antigen presentation. In chronic heart failure, the importance of activated monocytes, macrophages, DCs and T-cells has been increasingly recognized. However, the molecular mechanisms that are involved in pathological immune cell activation, and the specific roles of such alterations in the progression of adverse remodeling and inflammation in chronic heart failure, are unknown.

### SUMMARY

Disclosed herein are methods of modulating immune response in a subject using estrogen receptor beta (ERβ) agonists. The ERβ agonists can selectively inhibit the activation and/or proliferation of T-cells, reducing circulating T-cell levels in a subject without significantly affecting circulating levels of neutrophils, monocytes, or B-cells. As a result, the ERβ agonists can be used in therapeutic and/or prophylactic applications, including to treat or prevent chronic heart failure (CHF) in a subject post-myocardial infarction (MI) and to treat or prevent graft-versus-host disease (GVHD), multiple sclerosis (MS), and/or experimental autoimmune encephalomyelitis (EAE).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Gene expression for estrogen receptors (ERs) α and β (ERα and ERβ) in the ovaries (positive control), hearts from both males and females (M+F) and male spleens.

FIG. 2. Representative flow cytometric histograms for ERα (top panel) and ERβ (bottom panel) expression in different circulating and splenic immune cells in a male mouse.

FIG. 3. Representative flow cytometric histograms for cell trace violet labeled (CTV; a cell proliferation dye) CD4+ T-cells either unstimulated or CD3/CD28 TCR stimulated and treated with either Estradiol (5 and 50 nM) or Compound 1 (5 μM) or both are shown in FIG. 3. Peak patterns from high to low fluorescence intensity in stimulated cells represent halving of dye concentration in the cell membranes of the daughter cells with every successive cell division.

FIG. 4. Group quantitation for cell proliferation (%) measured as dye dilution with every successive cell division for stimulated groups. Mean values from 3-separate experiments conducted in quadruplicate by isolating splenic CD4+ T-cells from 3-male mice are reported. One way Anova was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 5. Group quantitation for cell proliferation (%) measured as dye dilution with every successive cell division for non-stimulated group. Mean values from 3-separate experiments conducted in quadruplicate by isolating splenic CD4+ T-cells from 3-male mice are reported. One way Anova was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 6. Cell survival of CD3/CD28 mediated in-vitro TCR stimulation with and without Compound 1 treatment. Mean values from 3-separate experiments conducted in quadruplicate by isolating splenic CD4+ T-cells from 3-male mice are reported. One way Anova was used for the data analysis.

FIG. 7. Representative flow cytometric histograms for TNFα expression in CD4+ T-cells either unstimulated or CD3/CD28 TCR stimulated and treated with either Estradiol (5 and 50 nM) or Compound 1 (5 μM) or both.

FIG. 8. Group quantitation for TNFα expressing CD4+ T-cells in stimulated groups. Mean values from 3-separate experiments conducted in quadruplicate by isolating splenic CD4+ T-cells from 3-male mice are reported. One way Anova was used for the data analysis. **p<0.01, and ****p<0.0001 represent significance with respect to non-stimulated group whereas ^{$}P<0.05, and ^{$$$}p<0.001 represent significance with respect to stimulated group without any other treatment.

FIG. 9. Group quantitation for TNFα expressing CD4+ helper T-cells in unstimulated control groups. Mean values from 3-separate experiments conducted in quadruplicate by isolating splenic CD4+ T-cells from 3-male mice are reported. One way Anova was used for the data analysis. **p<0.01, and ****p<0.0001 represent significance with respect to non-stimulated group whereas ^{$}P<0.05, and ^{$$$}p<0.001 represent significance with respect to stimulated group without any other treatment.

FIG. 10. Representative flow cytometric histograms for IFNγ expression in CD4+ T-cells either unstimulated or CD3/CD28 TCR stimulated and treated with either Estradiol (5 and 50 nM) or Compound 1 (5 μM) or both.

FIG. 11. Group quantitation for IFNγ expressing CD4+ T-cells in stimulated groups. Mean values from 3-separate experiments conducted in quadruplicate by isolating splenic CD4+ T-cells from 3-male mice are reported. One way Anova was used for the data analysis. *p<0.05 and **p<0.01 represent significance with respect to non-stimulated group.

FIG. 12. Group quantitation for IFNγ expressing CD4+ T-cells in unstimulated control groups. Mean values from 3-separate experiments conducted in quadruplicate by isolating splenic CD4+ T-cells from 3-male mice are reported. One way Anova was used for the data analysis. *p<0.05 and **p<0.01 represent significance with respect to non-stimulated group.

FIG. 13. Group quantitation for cell-survival of unstimulated or CD3/CD28 TCR stimulated T-cells isolated from female mice and treated with either Estradiol (5 and 50 nM) or Compound 1 (5 μM) or both. Mean values from 2-separate experiments conducted in quadruplicate by isolating splenic CD4+ T-cells from 2-female mice are reported. Statistical analysis could not be conducted on these data sets as these experiments were repeated from 2-mice only. However, T-cells from female mice exhibited similar trends as were observed with T-cells isolated from male mice.

FIG. 14. Group quantitation for proliferation of unstimulated or CD3/CD28 TCR stimulated and treated with either Estradiol (5 and 50 nM) or Compound 1 (5 μM) or both. Mean values from 2-separate experiments conducted in quadruplicate by isolating splenic CD4+ T-cells from 2-female mice are reported. Statistical analysis could not be conducted on these data sets as these experiments were repeated from 2-mice only. However, T-cells from female mice exhibited similar trends as were observed with T-cells isolated from male mice.

FIG. 15. Group quantitation for TNFα expressing CD4+ helper T-cells either unstimulated or CD3/CD28 TCR stimulated and treated with either Estradiol (5 and 50 nM) or Compound 1 (5 μM) or both. Mean values from 2-separate experiments conducted in quadruplicate by isolating splenic CD4+ T-cells from 2-female mice are reported. Statistical analysis could not be conducted on these data sets as these experiments were repeated from 2-mice only. However, T-cells from female mice exhibited similar trends as were observed with T-cells isolated from male mice.

FIG. 16. Group quantitation for IFNγ expressing CD4+ helper T-cells either unstimulated or CD3/CD28 TCR stimulated and treated with either Estradiol (5 and 50 nM) or Compound 1 (5 μM) or both. Mean values from 2-separate experiments conducted in quadruplicate by isolating splenic CD4+ T-cells from 2-female mice are reported. Statistical analysis could not be conducted on these data sets as these experiments were repeated from 2-mice only. However, T-cells from female mice exhibited similar trends as were observed with T-cells isolated from male mice.

FIG. 17. Group quantitation for cell-survival of CD4+ T-cells either unstimulated or stimulated with PMA/Ionomycin and treated with Compound 1 (5 μM). Mean values from 3-separate experiments conducted in quadruplicate by isolating splenic CD4+ T-cells from 3-male mice are reported. Two-way Anova was used for the data analysis.

FIG. 18. Group quantitation for TNFα expressing CD4+ helper T-cells either unstimulated or stimulated with PMA/Ionomycin and treated with Compound 1 (5 μM). Mean values from 3-separate experiments conducted in quadruplicate by isolating splenic CD4+ T-cells from 3-male mice are reported. Two-way Anova was used for the data analysis.

FIG. 19. Group quantitation for IFNγ expressing CD4+ helper T-cells either unstimulated or stimulated with PMA/Ionomycin and treated with Compound 1 (5 μM). Mean values from 3-separate experiments conducted in quadruplicate by isolating splenic CD4+ T-cells from 3-male mice are reported. Two-way Anova was used for the data analysis.

FIG. 20. Group quantitation for CD69+CD4+ activated helper T-cells either unstimulated or stimulated with PMA/Ionomycin and treated with Compound 1 (5 μM). Mean values from 3-separate experiments conducted in quadruplicate by isolating splenic CD4+ T-cells from 3-male mice are reported. Two-way Anova was used for the data analysis.

FIG. 21. Body weight (g) of sham-operated and myocardial infarction (MI) mice treated with either vehicle control or Compound 1. Treatment was started at 7 days post-infarction (designated as day 0 in the graph). For clarity of data, SD is shown only for heart failure group treated with vehicle and was comparable in all groups.

FIG. 22. Kaplan-Meier curve to show mortality rate in myocardial infarction and sham-operated mice treated with vehicle or drug. Treatment was started at 7 days post-myocardial infarction (designated as day 0 in both the graphs).

FIG. 23. Body weight (g) of sham-operated and myocardial infarction (MI) mice treated with either vehicle control or Compound 1. Treatment was started at 28 days post-infarction to inhibit immune activation in the chronic phase associated with left-ventricular remodeling. For clarity of data, SD is shown only for heart failure groups treated with either vehicle or the Compound 1 and was comparable in all groups.

FIG. 24. Tibia normalized heart weights (mg/mm) of sham-operated and myocardial infarction (MI) mice treated with either vehicle control or Compound 1. Treatment was started at 28 days post-infarction to inhibit immune activation in the chronic phase associated with left-ventricular remodeling. Two-way Anova was used for the data analysis: *p<0.05, and ***p<0.001.

FIG. 25. Levels of circulating CD4+ Helper T-cells (per μL blood) and its subsets viz CD4+Foxp3+(Tregs), CD4+TNFα+ cells, CD4+IFNγ+(Th1), CD4+IL-4+(Th2) and CD4+IL-17+(Th17) T-cells at 8 weeks post-surgery in mice treated with either vehicle or Compound 1 from 4 to at 8 weeks post-surgery. Two-way Anova was used for the data analysis.

FIG. 26. Quantitative group data for changes in left ventricular end-systolic volume (ESV, left panel), end-diastolic volume (EDV, middle panel), and ejection fraction (EF, right panel) in ligated mice before (4 weeks post-myocardial infarction) and after (8 weeks post-myocardial infarction) treatment with either vehicle or Compound 1. Students unpaired 2-tailed T-test was used for the data analysis.

FIG. 27. Schematic showing kinetics of CD4+ T-Cells in the myocardium at different time intervals post-myocardial infarction.

FIG. 28. Experimental design for study #1. An attrition rate of 10 and 40% for sham and heart failure groups is considered. LAD: Left-anterior descending coronary artery ligation.

FIG. 29. Experimental design for study #2. An attrition rate of 10 and 40% for sham and heart failure groups is considered.

FIG. 30: Schematic to show experimental protocol. At 8 weeks post-MI, CD4+ T-cells from the failing hearts (150 cells) and mediastinal lymph-nodes (300 cells) were flow sorted and the RNA sequencing was conducted to identify differential gene expression changes.

FIG. 31: IPA analysis identified SIRT1 activation as a positive upstream regulator in cardiac CD4+ T-cells vs lymph nodes. The strongest activation node downstream of SIRT1 was found to be ESR1 (ERα).

FIG. 32: Predicted ESR1 dependent gene expression changes in the dataset are indicated.

FIG. 33: Gene expression ERα and ERβ in female ovaries, hearts from males and females and spleens from males. *P<0.05, **P<0.01, ***p<0.001, and ****p<0.0001 represent significance with respect to groups shown.

FIG. 34: Gene expression of ERα and ERβ in splenic T-cells isolated from naive mice. *P<0.05, **P<0.01, ***p<0.001, and ****p<0.0001 represent significance with respect to groups shown.

FIG. 35: Representative flow cytometric histograms for ERβ expression in different circulating (left) and splenic (right) immune cells in male mice.

FIG. 36: Group quantitation for ERβ expression in different circulating (left) and splenic (right) immune cells in male mice. Data was analyzed using 1-way ANOVA with correction for multiple comparisons using Two-stage method of Benjamini, Krieger and Yekutieli by controlling the false discovery rate. *p<0.05, **p<0.01, ***p<0.001, and ****p<0.0001 represent significance with respect to CD19+ B-Cells, ^{$}P<0.05, ^{$$}p<0.01 and ^{$$$}p<0.001 represent significance with respect to CD4+ T-cells, ^{##}P<0.05 represent significance with respect to Ly6G+ neutrophils, and ^{@}P<0.05 represent significance with respect to Ly6C^{low} monocytes.

FIG. 37: Representative flow histograms showing ERα and ERβ expression in cardiac T-cells at 3 days post-MI.

FIG. 38: Group quantitation for ERα expression in cardiac T-cells at 3 d and at 8 w post-MI. Data was analyzed using two-tailed students T-test. *P<0.05 represents significance with respect to groups shown.

FIG. 39: Group quantitation for ERβ expression in cardiac T-cells at 3 d and at 8 w post-MI. Data was analyzed using two-tailed students T-test. *P<0.05 represents significance with respect to groups shown.

FIG. 40: Representative flow histograms showing ERβ expression in different splenic (left) and cardiac (right) immune cells at 3 d post-MI.

FIG. 41: Group quantitation for mean fluorescence intensity (MFI) of ERβ in CD19⁺ B-cells, CD4⁺ T-cells, CD11b⁺Ly6G⁺ neutrophils and CD11b⁺Ly6G⁻Ly6C⁺ monocytes in the spleens at 3d (left) and at 8 w post-MI (right). Data was analyzed using 1-way ANOVA with Tukey's post-hoc test. ****p<0.0001 represent significance with respect to CD19+ B-Cells, ^{$$}p<0.01, ^{$$$}p<0.001 and ^{$$$$}p<0.0001 represent significance with respect to CD4+ T-cells, ^{&&}P<0.01 represent significance with respect to Ly6C^{low} monocytes, and ^{####}P<0.01 represent significance with respect to Ly6G+ neutrophils.

FIG. 42: Group quantitation for mean fluorescence intensity (MFI) of ERβ in CD19⁺ B-cells, CD4⁺ T-cells, CD11b⁺Ly6G⁺ neutrophils and CD11b⁺Ly6G⁻Ly6C⁺ monocytes in the hearts at 3d (left) and at 8 w post-MI (right). Data was analyzed using 1-way ANOVA with Tukey's post-hoc test. ****p<0.0001 represent significance with respect to CD19+ B-Cells, ^{$$}p<0.01, ^{$$$}p<0.001 and ^{$$$$}p<0.0001 represent significance with respect to CD4+ T-cells, ^{&&}P<0.01 represent significance with respect to Ly6C^{low} monocytes, and ^{####}P<0.01 represent significance with respect to Ly6G+ neutrophils. *P<0.05, **P<0.01, and ***p<0.001 represent significance with respect to groups shown.

FIG. 43: Group quantitation for mean fluorescence intensity (MFI) of ERβ in CD4⁺ T-cells in the spleen, blood, and the hearts at 3 d. Data was analyzed using 1-way ANOVA with Tukey's post-hoc test. *P<0.05, **P<0.01, and ***p<0.001 represent significance with respect to groups shown.

FIG. 44: Group quantitation for mean fluorescence intensity (MFI) of ERβ in CD4⁺ T-cells in the spleen, blood, and the hearts at 8 w post-MI. Data was analyzed using 1-way ANOVA with Tukey's post-hoc test. *P<0.05, **P<0.01, and ***p<0.001 represent significance with respect to groups shown.

FIG. 45: ERβ expression (mean fluorescence intensity) in circulating, splenic, and cardiac CD19+ B-cells at 3d post-MI.

FIG. 46: Representative flow cytometric histograms for cell trace violet (CTV) labeled CD4+ T-cells either non-stimulated or stimulated with anti-CD3 and anti-CD28 antibodies in the absence and presence of different concentrations of compound 1. Peak patterns from high to low fluorescence intensity in stimulated cells represent halving of dye concentration in the cell membranes of the daughter cells with every successive cell division. Cell proliferation (%) in the presence of different concentrations of the drug is used to derive dose-response curve (lower panel, right).

FIG. 47: Cell proliferation (%) in non-stimulated CD4+ T-cells treated either with the vehicle control or estradiol (5 and 50 nM) in the presence or absence of compound 1. Mean±SD from 3-separate experiments conducted in triplicate by isolating splenic CD4+ T-cells from 3-male mice are reported. Data was analyzed using 1-way ANOVA with correction for multiple comparisons using Two-stage method of Benjamini, Krieger and Yekutieli by controlling the false discovery rate. **p<0.01, and ***p<0.001 represent significance with respect to non-stimulated group whereas ^{$}P<0.05 represent significance with respect to stimulated group treated with vehicle.

FIG. 48: Representative flow cytometric histograms for CTV labeled CD4+ T-cells either non-stimulated or stimulated with anti-CD3 and anti-CD28 antibodies and treated with either Estradiol (5 and 50 nM) or compound 1 (5 μM) or both.

FIG. 49: Group quantitation for % cell proliferation. Mean values from 3-separate experiments (conducted by isolating splenic CD4+ T-cells from 3-male mice) done in triplicate are reported. One way Anova with Tukey's post-hoc test was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 50: Frequency of live cells (% CD4) in stimulated (left) or non-stimulated (right) CD4+ T-cells treated either with the vehicle control or estradiol (5 and 50 nM) in the presence or absence of compound 1. Mean±SD from 3-separate experiments conducted in triplicate by isolating splenic CD4+ T-cells from 3-male mice are reported. Data was analyzed using 1-way ANOVA with correction for multiple comparisons using Two-stage method of Benjamini, Krieger and Yekutieli by controlling the false discovery rate. **p<0.01, and ***p<0.001 represent significance with respect to non-stimulated group whereas ^{$}P<0.05 represent significance with respect to stimulated group treated with vehicle.

FIG. 51: Representative flow histograms showing TNFα expression in non-stimulated and stimulated CD4+ T-cells treated either with estradiol or compound 1 or both. Mean values from 3-separate experiments (conducted by isolating splenic CD4+ T-cells from 3-male mice) done in triplicate are reported. One way Anova with Tukey's post-hoc test was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 52: Group quantitation for frequency of CD4+TNFα+ cells. Mean values from 3-separate experiments (conducted by isolating splenic CD4+ T-cells from 3-male mice) done in triplicate are reported. One way Anova with Tukey's post-hoc test was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 53: Group quantitation for frequency of CD4+IFNγ+ cells. Mean values from 3-separate experiments (conducted by isolating splenic CD4+ T-cells from 3-male mice) done in triplicate are reported. One way Anova with Tukey's post-hoc test was used for the data analysis. **P<0.01, ***p<0.001 and ****p<0.0001 represent significance with respect to non-stimulated group whereas ^{$$}P<0.01, ^{$$$}p<0.001 and ^{$$$$}p<0.0001 represent significance with respect to stimulated group.

FIG. 54: Expression of TNFα in non-stimulated CD4+ T-cells treated either with the vehicle control or estradiol (5 and 50 nM) in the presence or absence of compound 1. Mean±SD from 3-separate experiments conducted in triplicate by isolating splenic CD4+ T-cells from 3-male mice are reported. Data was analyzed using 1-way ANOVA with correction for multiple comparisons using Two-stage method of Benjamini, Krieger and Yekutieli by controlling the false discovery rate. **p<0.01, and ***p<0.001 represent significance with respect to non-stimulated group whereas ^{$}P<0.5 represent significance with respect to stimulated group treated with vehicle.

FIG. 55: Expression of IFNγ in non-stimulated CD4+ T-cells treated either with the vehicle control or estradiol (5 and 50 nM) in the presence or absence of compound 1. Mean±SD from 3-separate experiments conducted in triplicate by isolating splenic CD4+ T-cells from 3-male mice are reported. Data was analyzed using 1-way ANOVA with correction for multiple comparisons using Two-stage method of Benjamini, Krieger and Yekutieli by controlling the false discovery rate. **p<0.01, and ***p<0.001 represent significance with respect to non-stimulated group whereas ^{$}P<0.5 represent significance with respect to stimulated group treated with vehicle.

FIG. 56: Frequency CD69+ cells (% live cells) in stimulated CD4+ T-cells treated either with the vehicle, Estradiol (5 and 50 nM) or compound 1 drug (5 μM) or both. Mean±SD from 3-separate experiments conducted in triplicate by isolating splenic CD4+ T-cells from 3-male mice are reported. Data was analyzed using 1-way ANOVA with correction for multiple comparisons using Two-stage method of Benjamini, Krieger and Yekutieli by controlling the false discovery rate. **p<0.01, and ***p<0.001 represent significance with respect to non-stimulated group whereas ^{$}P<0.5 represent significance with respect to stimulated group treated with vehicle.

FIG. 57: Group quantitation for live cells (% CD4) either unstimulated or CD3/CD28 TCR stimulated and treated with either Estradiol (5 and 50 nM) or compound 1 drug (5 μM) or both. Mean values from 2-separate experiments conducted in quadruplicate by isolating splenic CD4+ T-cells from 2 female mice are reported. Data was analyzed using 1-way ANOVA with correction for multiple comparisons using Two-stage method of Benjamini, Krieger and Yekutieli by controlling the false discovery rate. *p<0.05, **p<0.01 ***p<0.001, and ****p<0.0001 represent significance with respect to vehicle treated non-stimulated group whereas ^{$}P<0.5, ^{$$}P<0.01, and ^{$$$}P<0.001 represent significance with respect to vehicle treated stimulated group.

FIG. 58: Group quantitation for proliferation (% live cells) either unstimulated or CD3/CD28 TCR stimulated and treated with either Estradiol (5 and 50 nM) or compound 1 drug (5 μM) or both. Mean values from 2-separate experiments conducted in quadruplicate by isolating splenic CD4+ T-cells from 2 female mice are reported. Data was analyzed using 1-way ANOVA with correction for multiple comparisons using Two-stage method of Benjamini, Krieger and Yekutieli by controlling the false discovery rate. *p<0.05, **p<0.01 ***p<0.001, and ****p<0.0001 represent significance with respect to vehicle treated non-stimulated group whereas ^{$}P<0.5, ^{$$}P<0.01, and ^{$$$}P<0.001 represent significance with respect to vehicle treated stimulated group.

FIG. 59: Group quantitation for TNFα+ either unstimulated or CD3/CD28 TCR stimulated and treated with either Estradiol (5 and 50 nM) or compound 1 drug (5 μM) or both. Mean values from 2-separate experiments conducted in quadruplicate by isolating splenic CD4+ T-cells from 2 female mice are reported. Data was analyzed using 1-way ANOVA with correction for multiple comparisons using Two-stage method of Benjamini, Krieger and Yekutieli by controlling the false discovery rate. *p<0.05, **p<0.01 ***p<0.001, and ****p<0.0001 represent significance with respect to vehicle treated non-stimulated group whereas ^{$}P<0.5, ^{$$}P<0.01, and ^{$$$}P<0.001 represent significance with respect to vehicle treated stimulated group.

FIG. 60: Group quantitation for IFNγ+ helper CD4+ T-cells either unstimulated or CD3/CD28 TCR stimulated and treated with either Estradiol (5 and 50 nM) or compound 1 drug (5 μM) or both. Mean values from 2-separate experiments conducted in quadruplicate by isolating splenic CD4+ T-cells from 2 female mice are reported. Data was analyzed using 1-way ANOVA with correction for multiple comparisons using Two-stage method of Benjamini, Krieger and Yekutieli by controlling the false discovery rate. *p<0.05, **p<0.01 ***p<0.001, and ****p<0.0001 represent significance with respect to vehicle treated non-stimulated group whereas ^{$}P<0.5, ^{$$}P<0.01, and ^{$$$}P<0.001 represent significance with respect to vehicle treated stimulated group.

FIG. 61: Group quantitation for cell-survival either unstimulated or stimulated with PMA/Ionomycin and treated with compound 1 drug (5 PM). Mean values from 3-separate experiments conducted in quadruplicate by isolating splenic CD4+ T-cells from 3-male mice are reported.

FIG. 62: Group quantitation for TNFα+ either unstimulated or stimulated with PMA/Ionomycin and treated with compound 1 drug (5 μM). Mean values from 3-separate experiments conducted in quadruplicate by isolating splenic CD4+ T-cells from 3-male mice are reported. Two-way Anova with Tukey's post-hoc test was used for the data analysis and respective p-values are shown. *P<0.05, **P<0.01, ***p<0.001, and ****p<0.0001 represent significance with respect to groups shown.

FIG. 63: Group quantitation for IFNγ+ either unstimulated or stimulated with PMA/Ionomycin and treated with compound 1 drug (5 μM). Mean values from 3-separate experiments conducted in quadruplicate by isolating splenic CD4+ T-cells from 3-male mice are reported. Two-way Anova with Tukey's post-hoc test was used for the data analysis and respective p-values are shown. *P<0.05, **P<0.01, ***p<0.001, and ****p<0.0001 represent significance with respect to groups shown.

FIG. 64: Group quantitation for CD69+ helper T-cells (CD4+) either unstimulated or stimulated with PMA/Ionomycin and treated with compound 1 drug (5 μM). Mean values from 3-separate experiments conducted in quadruplicate by isolating splenic CD4+ T-cells from 3-male mice are reported. Two-way Anova with Tukey's post-hoc test was used for the data analysis and respective p-values are shown. *P<0.05, **P<0.01, ***p<0.001, and ****p<0.0001 represent significance with respect to groups shown.

FIG. 65: Principal Component Analysis of RNA transcriptomes of naïve and stimulated CD4+ T-cells treated either with the vehicle (DMSO) or compound 1 (5 PM).

FIG. 66: Volcano plot showing several genes (marked as red) are either upregulated or downregulated by more than 2-fold in stimulated CD4+ T-cells treated with compound 1 (5 μM). Some of the representative genes that showed either very high Log P values or very high fold-changes are shown.

FIG. 67: Ingenuity Pathway analysis of RNA transcriptomes of stimulated CD4+ T-cells treated either with the vehicle control (DMSO) or compound 1 (5 PM) to show activation of ERβ and downregulation of ERα pathway demonstrating specificity of the drug.

FIG. 68: Heat maps depicting genes in the ERβ pathway that are either significantly upregulated or downregulated in stimulated CD4+ T-cells upon treatment with compound 1 (5 μM).

FIG. 69: Heat maps depicting genes in the TCR pathway that are either significantly upregulated or downregulated in stimulated CD4+ T-cells upon treatment with compound 1 (5 μM).

FIG. 70: Depiction of genes that are either significantly upregulated or downregulated in stimulated CD4+ T-cells upon treatment with compound 1 (5 μM).

FIG. 71: Schematic for the experimental plan to test the efficacy of compound 1 during acute phase of MI and during chronic HF.

FIG. 72: Body weight (g) of sham-operated and myocardial infarction (MI) mice treated either with the vehicle control or compound 1 (60 mg/kg/day; gavage). For clarity of data, SD is shown only for HF group treated with vehicle and was comparable in all groups.

FIG. 73: Kaplan-Meier curve to show mortality rate in MI and sham-operated mice treated with the vehicle or drug. Treatment was started at 7d post-MI (designated as day 0 in FIG. 72 and FIG. 73 graphs).

FIG. 74: End-systolic and end-diastolic volumes (ESV and EDV), and ejection fraction (EF) of mice at 4 w post-MI.

FIG. 75: Body weight (g) of sham-operated and MI mice treated either with the vehicle control or compound 1. For clarity of data, SD is shown only for both the HF groups and was comparable in all groups.

FIG. 76: Representative B-mode tracings depicting systole and diastole of failing hearts at 4 w (at the time of randomization) and 8 w post-MI after treatment with either the vehicle or the drug.

FIG. 77: Group quantitation for the change in end-systolic and end-diastolic volumes (ESV and EV), and the ejection fraction (EF) from 4 to 8 w post-MI after treatment with either the vehicle or the drug. Unpaired students two-tailed T-test was used for analyzing data. *P<0.05 and ****p<0.0001 represent significance with respect to groups shown.

FIG. 78: Heart rate (BPM) of mice treated either with the vehicle control or compound 1 at 4 and 8 w post-MI.

FIG. 79: Gravimetric data for tibia normalized heart weights of sham and HF mice treated either with the vehicle or compound 1. Two way Anova with Tukey's post-hoc test was used for data analysis. *P<0.05, **P<0.01, ***p<0.001 and ****p<0.0001 represent significance with respect to groups shown.

FIG. 80: Gravimetric data for tibia normalized LV weights of sham and HF mice treated either with the vehicle or compound 1. Two way Anova with Tukey's post-hoc test was used for data analysis. *P<0.05, **P<0.01, ***p<0.001 and ****p<0.0001 represent significance with respect to groups shown.

FIG. 81: Representative images of LV sections stained with FITC conjugated Wheat-germ agglutinin to show cardiac hypertrophy. Boxed area in the upper panel is shown at its original magnification in the lower panel.

FIG. 82: Group quantitation for cardiomyocyte area. Two-tailed students T-test was used for data analysis. *P<0.05, **P<0.01, ***p<0.001 and ****p<0.0001 represent significance with respect to groups shown.

FIG. 83: Gene expression of cardiac hypertrophy markers in the remote-zone LV of HF mice treated either with the vehicle or compound 1 from 4 to 8 w post-MI. Two-tailed students T-test was used for data analysis. *P<0.05, **P<0.01, ***p<0.001 and ****p<0.0001 represent significance with respect to groups shown.

FIG. 84; Heat map depicting cardiac hypertrophy genes in stimulated CD4+ T-cells upon treatment with compound 1 (5 μM).

FIG. 85: Representative flow scatter plots for CD4+ and CD8+ T-cells and quantitative data for circulating CD4+ Helper T-cells (/μL blood) and its subsets viz CD4+TNFα+ cells, CD4+Foxp3+(Tregs), CD4+IFNγ+(Th1), CD4+IL-4+(Th2) and CD4+IL-17+(Th17) T-cells at 8 w post-MI in mice treated either with vehicle or compound 1 from 4 to 8 w post-surgery. Unpaired students two-tailed T-test was used for data analysis, and *p<0.05, **p<0.01 and ***p<0.001, and ****p<0.0001 were considered significant.

FIG. 86: Representative flow scatter plots depicting CD4+ and CD8+ T-cells in total CD45+ cells.

FIG. 87: Levels of CD4+ Helper T-cells and its pro-inflammatory subsets viz CD4+TNFα+ cells and CD4+IFNγ+(Th1), T-cells at 8 weeks post-MI in mice treated either with the vehicle or compound 1 from 4 to at 8 w post-MI. Unpaired Students 2-tailed T-test was used for the data analysis. *P<0.05, **P<0.01, and ***p<0.001 represent significance with respect to groups shown.

FIG. 88: Representative flow scatter plots for splenic CD4+ and CD8+ T-cells in CD45+ leukocytes.

FIG. 89: Group quantitation for splenic CD4+ Helper T-cells (total cells and frequency) at 8 w post-MI in mice treated either with the vehicle or compound 1 drug from 4 to 8 w post-surgery. Unpaired Students 2-tailed T-test was used for the data analysis. *P<0.05, **P<0.01, and ***p<0.001 represent significance with respect to groups shown.

FIG. 90: Representative flow scatter plots for splenic CD4+ and FoxP3+ T-cells.

FIG. 91: Quantitative data for splenic CD4+FoxP3+ regulatory T-cells (total cells) at 8 w post-MI in mice treated either with vehicle or compound 1 from 4 to at 8 w post-surgery. Students 2-tailed T-test was used for the data analysis and *p<0.05 was considered significant.

FIG. 92: Quantitative data for splenic CD4+FoxP3+ regulatory T-cells (frequency) at 8 w post-MI in mice treated either with vehicle or compound 1 from 4 to at 8 w post-surgery. Students 2-tailed T-test was used for the data analysis and *p<0.05 was considered significant.

FIG. 93: Quantitative data for FoxP3 MFI (protein expression) at 8 w post-MI in mice treated either with vehicle or compound 1 from 4 to at 8 w post-surgery. Students 2-tailed T-test was used for the data analysis and *p<0.05 was considered significant.

FIG. 94: ERβ MFI (protein expression) in CD4+ and CD8+ T-cells (upper), and in different CD4+ helper T-cell subsets viz Tregs, Th1, and Th17 T-cells at 8 weeks post-surgery in mice treated either with vehicle or compound 1 drug from 4 to at 8 weeks post-surgery. Students 2-tailed T-test was used for the data analysis.

FIG. 95: Quantitative data (frequency) for cardiac CD11b+ myeloid cells, CD11b+Ly6G+ neutrophils, CD11b+Ly6G−Ly6C+ monocytes (Ly6C^{high} pro-inflammatory and Ly6C^{low} patrolling), CD19+ B-cells and CD8+ T-cells at 8 w post-surgery in mice treated either with vehicle or compound 1 drug from 4 to 8 w post-surgery. Students 2-tailed T-test was used to compare each cell type.

FIG. 96: Quantitative data (frequency) for circulating CD11b+ myeloid cells, CD11b+Ly6G+ neutrophils, CD11b+Ly6G−Ly6C+ monocytes (Ly6C^{high} pro-inflammatory and Ly6C^{low} patrolling), CD19+ B-cells and CD8+ T-cells at 8 w post-surgery in mice treated either with vehicle or compound 1 drug from 4 to 8 w post-surgery. Students 2-tailed T-test was used to compare each cell type.

FIG. 97: Quantitative data (frequency) for splenic CD11b+ myeloid cells, CD11b+Ly6G+ neutrophils, CD11b+Ly6G−Ly6C+ monocytes (Ly6C^{high} pro-inflammatory and Ly6C^{low} patrolling), CD19+ B-cells and CD8+ T-cells at 8 w post-surgery in mice treated either with vehicle or compound 1 drug from 4 to 8 w post-surgery. Students 2-tailed T-test was used to compare each cell type.

FIG. 98: Tibia normalized thymus weights at 8 weeks post-surgery in mice treated either with vehicle or compound 1 drug from 4 to 8 weeks post-surgery. Students 2-tailed T-test was used to compare each cell type.

FIG. 99: Cell counts (left) and frequency (right) of single positive double negative (DN; CD4−CD8−), single positive (SP; CD4+CD8− and CD4−CD8+), and double positive (DP; CD4+CD8+) T-cells in thymus at 8 weeks post-surgery in mice treated either with vehicle or compound 1 drug from 4 to 8 weeks post-surgery. Students 2-tailed T-test was used to compare each cell type.

FIG. 100: Double negative (DN) T-cells were further separated into DN1 (CD44+CD25−), DN2 (CD44+CD25+), DN3 (CD44−CD25+), and DN4 (CD44−CD25−) T-cells at 8 weeks post-surgery in mice treated either with vehicle or compound 1 drug from 4 to 8 weeks post-surgery. Students 2-tailed T-test was used to compare each cell type.

### DETAILED DESCRIPTION

The compounds, compositions, and methods described herein may be understood more readily by reference to the following detailed description of specific aspects of the disclosed subject matter and the Examples included therein.

Before the present compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of fibrotic conditions, “treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

“Therapeutically effective amount,” as used herein, refers to a minimal amount or concentration of an ERβ agonist that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the fibrotic condition is encompassed by this term.

As used herein, unless otherwise specified, the terms “prevent,” “preventing” and “prevention” refers to an action that occurs before the subject begins to suffer from the condition, or relapse of such condition. The prevention need not result in a complete prevention of the condition; partial prevention or reduction of the fibrotic condition is encompassed by this term.

As used herein, unless otherwise specified, a “prophylactically effective amount” of an ERβ that, when administered alone or in combination, prevent the condition, or one or more symptoms associated with the condition, or prevent its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of the fibrotic condition is encompassed by this term.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

ERβ Agonists

Described herein are methods of treating and preventing fibrotic conditions using estrogen receptor beta (ERβ) agonists. In some examples, the agonist can have an EC₅₀ of 800 nM or less at estrogen receptor beta (ERβ) (e.g., 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 9 nM or less, 8 nM or less, 7 nM or less, 6 nM or less, 5 nM or less, 4.5 nM or less, 4 nM or less, 3.5 nM or less, 3 nM or less, 2.5 nM or less, 2 nM or less, 1.5 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less).

In some examples, the agonist can have an EC₅₀ of 1 μM or more at ERβ (e.g., 0.1 nM or more, 0.2 nM or more, 0.3 nM or more, 0.4 nM or more, 0.5 nM or more, 0.6 nM or more, 0.7 nM or more, 0.8 nM or more, 0.9 nM or more, 1 nM or more, 1.5 nM or more, 2 nM or more, 2.5 nM or more, 3 nM or more, 3.5 nM or more, 4 nM or more, 4.5 nM or more, 5 nM or more, 6 nM or more, 7 nM or more, 8 nM or more, 9 nM or more, 10 nM or more, 20 nM or more, 30 nM or more, 40 nM or more, 50 nM or more, 60 nM or more, 70 nM or more, 80 nM or more, 90 nM or more, 100 nM or more, 200 nM or more, 300 nM or more, 400 nM or more, 500 nM or more, 600 nM or more, or 700 nM or more).

The EC₅₀ of the agonist at ERβ can range from any of the minimum values described above to any of the maximum values described above. For example, the compounds disclosed herein can have an EC₅₀ of from 1 μM to 800 nM at ERβ (e.g., from 1 pM to 400 nM, from 400 nM to 800 nM, from 1 pM to 300 nM, from 1 pM to 200 nM, from 1 pM to 100 nM, from 1 pM to 50 nM, from 1 pM to 20 nM, from 1 pM to 10 nM, from 1 pM to 6 nM, from 1 pM to 5 nM, from 1 pM to 2 nM, from 1 pM to 1 nM, from 1 pM to 0.7 nM, from 1 pM to 0.5 nM, from 1 pM to 0.2 pM, or from 1 pM to 0.1 nM).

In some examples, the agonist can be a selective ERβ agonist. In some examples, a selective ERβ agonist is a compound that has a lower EC₅₀ at ERβ than at estrogen receptor α (ERα). The selectivity of the agonists can, in some examples, be expressed as an ERβ-to-ERα agonist ratio, which is the EC₅₀ of the compound at ERα divided by the EC₅₀ of the compound at ERβ. In some examples, the agonists can have an ERβ-to-ERα agonist ratio of 8 or more (e.g., 10 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more, 1000 or more, 1100 or more, 1200 or more, 1300 or more, 1400 or more, 1500 or more, 2000 or more, 2500 or more).

In some examples, the agonists can have an ERβ-to-ERα agonist ratio of 3000 or less (e.g., 2500 or less, 2000 or less, 1500 or less, 1400 or less, 1300 or less, 1200 or less, 1100 or less, 1000 or less, 900 or less, 800 or less, 700 or less, 600 or less, 500 or less, 450 or less, 400 or less, 350 or less, 300 or less, 250 or less, 200 or less, 150 or less, 100 or less, 90 or less, 80 or less, 70 or less, 60 or less, 50 or less, 40 or less, 30 or less, 20 or less, or 10 or less).

The ERβ-to-ERα agonist ratio of the compounds at ERβ can range from any of the minimum values described above to any of the maximum values described above. For example, the compounds can have an ERβ-to-ERα agonist ratio of from 8 to 3000 (e.g., from 8 to 1500, from 1500 to 3000, from 400 to 3000, from 500 to 3000, from 600 to 3000, from 700 to 3000, from 800 to 3000, from 900 to 3000, from 1000 to 3000, or from 2000 to 3000).

Numerous ERβ agonists are known in the art, and described for example in Mohler, M. L., et al “Estrogen Receptor β Selective Nonsteroidal Estrogens: Seeking Clinical Indications” Expert Opin. Ther. Patents (2010) 20(4): 507-534, which is incorporated herein by reference.

In some embodiments, the agonist can be a hydroxy-biphenyl-carbaldehyde oxime derivative. Examples of such agonists are described, for example, in U.S. Pat. No. 7,279,600 to Mewshaw et al. and International Publication No. WO 2004/099122 to Mewshaw et al., each of which is hereby incorporated by reference in its entirety.

In some embodiments, the agonist can be defined by Formula I below

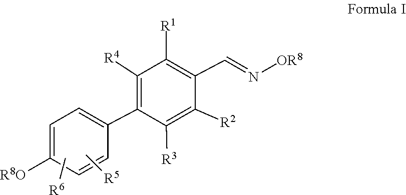

wherein

R¹ and R², are each, independently, H, halogen, CN, substituted or unsubstituted phenyl, or substituted or unsubstituted alkyl (e.g., substituted or unsubstituted C₁-C₆ alkyl);

R³, R⁴, R⁵ and R⁶, are each independently, H, OH, halogen, CN, substituted or unsubstituted phenyl, substituted or unsubstituted alkyl (e.g., substituted or unsubstituted C₁-C₆ alkyl), or substituted or unsubstituted alkoxy (e.g., substituted or unsubstituted C₁-C₆ alkoxy);

R⁸ are each, independently H, —C(O)R⁹, or substituted or unsubstituted alkyl (e.g., substituted or unsubstituted C₁-C₆ alkyl); and

R⁹ is substituted or unsubstituted alkyl (e.g., substituted or unsubstituted C₁-C₆ alkyl);

or a pharmaceutically acceptable salt thereof or a prodrug thereof.

In some embodiments, R⁸ is, in each case, H.

In certain embodiments, the agonist can be one of the compounds shown below.

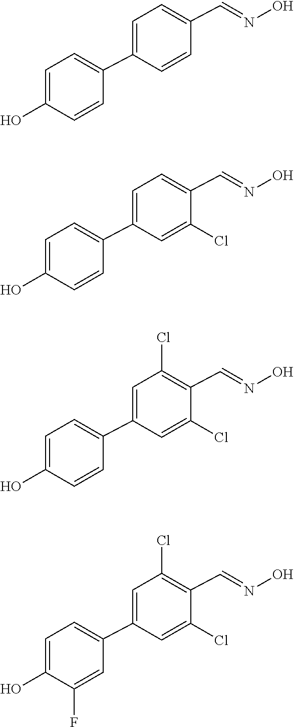 [SMILES: O/N=C/c1ccc(-c2ccc(O)cc2)cc1 ; O/N=C/c1ccc(-c2ccc(O)cc2)cc1Cl ; O/N=C/c1c(Cl)cc(-c2ccc(O)cc2)cc1Cl ; O/N=C/c1c(Cl)cc(-c2ccc(O)c(F)c2)cc1Cl]

In some embodiments, the agonist can be a naphthyl-linked carbaldehyde oxime derivative. Examples of such agonists are described, for example, in U.S. Pat. No. 7,157,491 to Mewshaw et al. and International Publication No. WO 2004/103941 to Mewshaw et al., each of which is hereby incorporated by reference in its entirety.

In some embodiments, the agonist can be defined by Formula II below

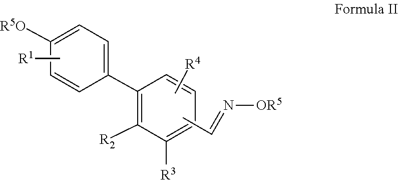

wherein

R¹ is hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy);

R² and R³, together, form a fused aryl or heteroaryl ring;

R⁴ is hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy);

R⁵ are each, independently H, —C(O)R⁶, or substituted or unsubstituted alkyl (e.g., substituted or unsubstituted C₁-C₆ alkyl); and

R⁶ is substituted or unsubstituted alkyl (e.g., substituted or unsubstituted C₁-C₆ alkyl);

or a pharmaceutically acceptable salt thereof or a prodrug thereof.

In some embodiments, R⁵ is, in each case, H.

In certain embodiments, the agonist can be one of the compounds shown below.

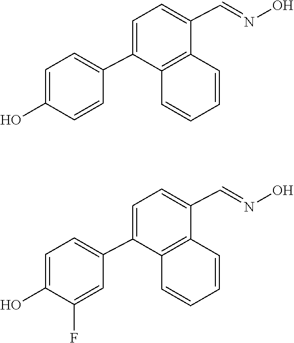 [SMILES: O/N=C/c1ccc(-c2ccc(O)cc2)c2ccccc12 ; O/N=C/c1ccc(-c2ccc(O)c(F)c2)c2ccccc12]

In some embodiments, the agonist can be an indole-linked carbaldehyde oxime derivative. Examples of such agonists are described, for example, in U.S. Pat. No. 7,250,440 to Mewshaw et al. and International Publication No. WO 2005/018636 to Mewshaw et al., each of which is hereby incorporated by reference in its entirety.

In some embodiments, the agonist can be defined by Formula III below

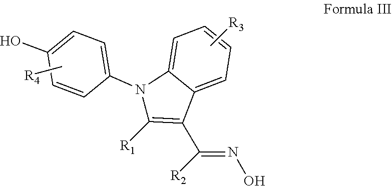

wherein

R₁ is hydrogen, substituted or unsubstituted alkyl (e.g., substituted or unsubstituted C₁-C₆ alkyl), halogen, CN, or substituted or unsubstituted alkoxy (e.g., substituted or unsubstituted C₁-C₆ alkoxy) and R² is hydrogen, substituted or unsubstituted alkyl (e.g., substituted or unsubstituted C₁-C₆ alkyl), or substituted or unsubstituted phenyl; or R₁ and R₂ together may form a 5-7 membered ring; and

R₃, and R₄ are each, independently, H, OH, halogen, CN, substituted or unsubstituted phenyl, substituted or unsubstituted alkyl (e.g., substituted or unsubstituted C₁-C₆ alkyl), or substituted or unsubstituted alkoxy (e.g., substituted or unsubstituted C₁-C₆ alkoxy);

or a pharmaceutically acceptable salt or prodrug thereof.

In certain embodiments, the agonist can be one of the compounds shown below.

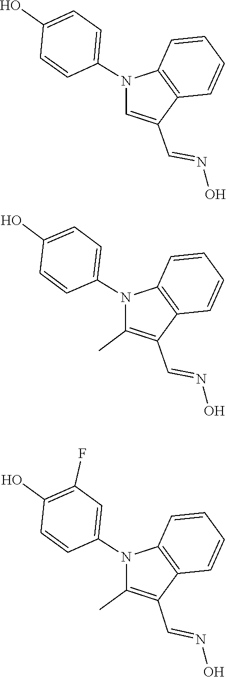

In some embodiments, the agonist can be defined by Formula IV below

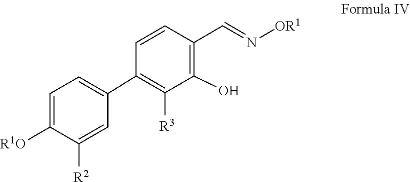

wherein

R¹ is, independently for each occurrence, H, —C(O)R⁴, or substituted or unsubstituted alkyl (e.g., substituted or unsubstituted C₁-C₆ alkyl); and

R² is hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy);

R³ is hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy); and

R⁴ is substituted or unsubstituted alkyl (e.g., substituted or unsubstituted C₁-C₆ alkyl);

or a pharmaceutically acceptable salt thereof or a prodrug thereof.

In some embodiments, R¹ is, in each case, H.

In certain embodiments, the agonist can be one of the compounds shown below.

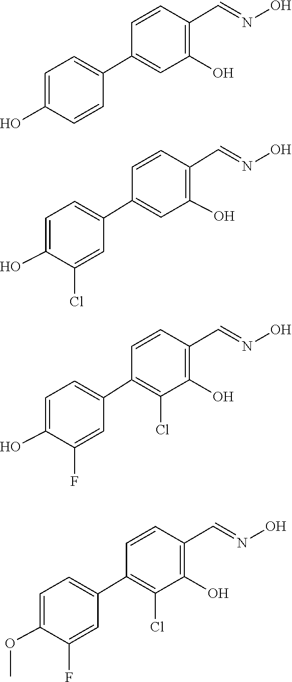 [SMILES: O/N=C/c1ccc(-c2ccc(O)cc2)cc1O ; O/N=C/c1ccc(-c2ccc(O)c(Cl)c2)cc1O ; O/N=C/c1ccc(-c2ccc(O)c(F)c2)c(Cl)c1O ; COc1ccc(-c2ccc(/C=N/O)c(O)c2Cl)cc1F]

In some embodiments, the agonist can comprise 2,3-bis(4-hydroxyphenyl)propionitrile (DPN) or a derivative or analog thereof. For example, in some embodiments, the agonist can be a compound defined by Formula V below

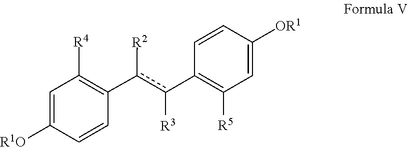

wherein

the dashed line indicates a single bond or a double bond;

R¹ is, independently for each occurrence, H, —C(O)R⁶, or substituted or unsubstituted alkyl (e.g., substituted or unsubstituted C₁-C₆ alkyl);

R² and R³ are each, independently, H or CN;

R⁴ and R⁵ are each, independently, hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy); and

R⁶ is substituted or unsubstituted alkyl (e.g., substituted or unsubstituted C₁-C₆ alkyl);

or a pharmaceutically acceptable salt thereof or a prodrug thereof.

In some embodiments, R¹ is, in each case, H.

In some embodiments, the agonist can be a compound defined by the formula below

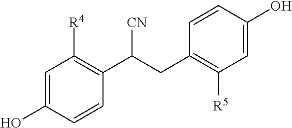

wherein R⁴ and R⁵ are each, independently, hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy). In one embodiment, R⁴ and R⁵ are each, independently, hydrogen or methyl.

In some embodiments, the agonist can be a compound defined by the formula below.

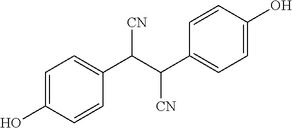 [SMILES: N#CC(c1ccc(O)cc1)C(C#N)c1ccc(O)cc1]

In some embodiments, the agonist can be a compound defined by the formula below

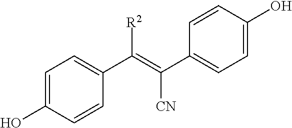

wherein R⁴ is hydrogen or CN.

In some embodiments, the agonist can be a sulfonamide. Examples of such agonists are described, for example, in U.S. Patent Application Publication No. 20070021495 to Katzenellenbogen et al., which is hereby incorporated by reference in its entirety.

In some embodiments, the agonist can be defined by Formula VI below

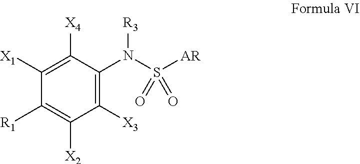

or a salt, stereoisomer or prodrug thereof wherein

AR is an optionally substituted aryl group;

R₃ is an alkyl, alkenyl, alkynyl, benzyl, or phenyl group;

R₁ is a hydrogen, a halide, a hydroxy, thiol, an alkyl, alkenyl, alkynyl, benzyl, phenyl alkoxy, thioalkoxy, or aryloxy group; and

X₁-X₄, independently of one another, are selected from the group consisting of hydrogens, halogens, alkyl groups, alkoxy groups, —CO—R groups, —SR groups, cyano groups, nitro groups, hydroxy groups, alkoxy groups, thiol groups, and thioalkoxy groups, where R is H, or an alkyl group, wherein R₃ can be linked with X₃, or X₄ to form a 5, 6 or 7-member ring which may be an aromatic ring, or may contain one or two double bonds and wherein the ring optionally contains one or two additional heteroatoms wherein all alkyl, alkenyl, alkynyl, aryl, benzyl and phenyl groups are optional substituted and wherein optional substitution means substitution with one or more halogens, cyano groups, nitro groups, hydroxy groups, alkoxy groups, thiol groups, thioalkoxy groups, aryloxy groups, N(R)′₂ groups, CON(R′)₂ groups or —COOR′ groups, where R′ is H or an alkyl group and where R′ groups may be linked to form a cyclic alkyl group.

In certain embodiments, the agonist can be defined by the formula below

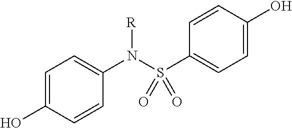

wherein R is substituted or unsubstituted alkyl (e.g., substituted or unsubstituted C₁-C₆ alkyl, such as —CH₃, —CH₂CH₂CH₃, —CH₂CH₂CH₂CH₃, —CH(CH₃)(CH₂CH₃), —CH(CH₃)(CH₂CH₂CH₃), or —CH₂CH₂CF₃).

In some embodiments, the agonist can comprise a monocycle-linked bis-phenyl estrogenic agonist. Examples of such agonists are described, for example, in International Publication No. WO 2000/019994 to Katzenellenbogen et al., which is hereby incorporated by reference in its entirety.

In some embodiments, the agonist can comprise a thiophene-based agonist. Examples of such agonists are described, for example, in U.S. Pat. No. 6,835,745 to Coghlan et al., which is hereby incorporated by reference in its entirety.

In some embodiments, the agonist can be defined by Formula VII below

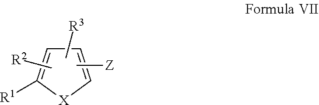

wherein

R¹ is phenyl optionally substituted with 1-4 Y groups;

R² is phenyl optionally substituted with 1-4 Y groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;

R³ is hydrogen, phenyl optionally substituted with 1-4 Y groups, alkyl of 1-6 carbon atoms, alkoxy of 1-6 carbon atoms, alkoxycarbonyl of 2-7 carbon atoms, alkylthio of 1-6 carbon atoms, haloalkyl of 1-6 carbon atoms, alkenyl of 2-7 carbon atoms, alkynyl of 2-7 carbon atoms, haloalkenyl of 2-7 carbon atoms, or haloalkynyl of 2-7 carbon atoms;

X is O, —CH═CH—, or S;

Y is —OH, —OR⁴, halogen, —CN, —CO₂H, —CO₂R⁴, alkyl of 1-6 carbon atoms, alkenyl of 2-7 carbon atoms, alkynyl of 2-7 carbon atoms, perfluoroalkyl of 1-6 carbon atoms, or —COR⁴;

Z is —CHO, —CN, —CO₂H, —CO₂R⁴, —CONR⁴R⁵, —NO₂, —CH═NR⁴, —CH═—CH═NOR⁴;

R⁴ and R⁵ are each, independently, alkyl of 1-6 carbon atoms, alkenyl of 2-7 carbon atoms, alkynyl of 2-7 carbon atoms, or cycloalkyl of 3-8 carbon atoms;

with the proviso that at least one of R² or R³ is phenyl or phenyl substituted with 1-4 Y groups;

or a pharmaceutically acceptable salt thereof.

In some embodiments, the agonist can be a compound defined by the formula below

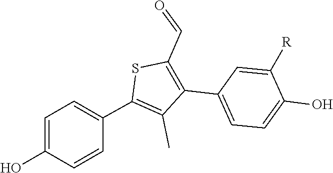

wherein R is hydrogen, halogen, substituted or unsubstituted alkyl (e.g., substituted or unsubstituted C₁-C₆ alkyl), CN, or substituted or unsubstituted alkoxy (e.g., substituted or unsubstituted C₁-C₆ alkoxy). In one embodiment, R is hydrogen or halogen (e.g., F or Cl).

In some embodiments, the agonist can comprise a cycloalkane-linked biphenyl. Examples of such agonists are described, for example, in U.S. Patent Application Publication No. 2005/0256210 to Olsson et al., which is hereby incorporated by reference in its entirety. In some embodiments, the agonist can be defined by Formula VIII below

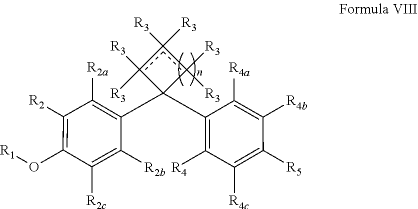

or a pharmaceutically acceptable salt or prodrug thereof, wherein:

n is an integer selected from the group consisting of 3, 4, 5 and 6;

R₁ is selected from the group consisting of hydrogen, C₁-C₈ straight chained or branched alkyl, C₁-C₈ straight chained or branched alkenyl, cycloalkyl, cycloalkenyl, substituted or unsubstituted heteroaryl, substituted or unsubstituted aryl, substituted or unsubstituted heteroalicyclyl, sulphonyl, C₁-C₈ straight chained or branched perhaloalkyl, —C(═Z)R₆, —C(═Z)OR₆, and —C(═Z)N(R₆)₂;

R₂, R₂ₐ, R_{2b}, R_{2c} are separately selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, halogen, sulfonyl, perhaloalkyl, —CN, —OR₆, —NR₆R₆ₐ, —NR₆NR₆ₐR_{6b}, —NR₆N═CR₆ₐR_{6b}, —N(R₆)C(R₆ₐ)═NR_{6b}, —C(═Z)R₆, —C(═Z)OR₆, —C(═Z)NR₆R₆ₐ, —N(R₆)—C(═Z)R₆ₐ, —N(R₆)—C(═Z)NR_{6b}R₆ₐ, —OC(═Z)R₆, —N(R₆)—S(═O)₂R₆ₐ, and —SR₆;

each R₃ is separately selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, halogen, sulfonyl, perhaloalkyl, —CN, ═O, and —OR₆, or are separately absent to accommodate a double bond;
two R₃ groups are optionally bound together to form a substituted or unsubstituted C₃-C₉ cycloalkyl or C₃-C₉ heteroalicyclyl;

any bond represented by a dashed and solid line represents a bond selected from the group consisting of a single bond and a double bond;

R₄, R₄ₐ, R_{4b}, R_{4c} are separately selected from the group consisting of hydrogen, alkyl, alkenyl, cycloalkyl, cycloalkenyl, substituted or unsubstituted aryl, substituted or unsubstituted heteroaryl, substituted or unsubstituted heteroalicyclyl, hydroxy, nitro, halogen, sulfonyl, perhaloalkyl, —OR₆, —NR₆R₆ₐ, —NR₆NR₆ₐR_{6b}, —NR₆N═CR₆ₐR_{6b}, —N(R₆)C(R₆ₐ)═NR_{6b}, —CN, —C(═Z)R₆, —C(═Z)OR₆, —C(═Z)NR₆R₆ₐ, —S(═Z)NR₆R₆ₐ, —N(R₆)—C(═Z)R₆ₐ, —N(R₆)—C(═Z)NR_{6b}R₆ₐ, —OC(═Z)R₆, —N(R₆)—S(═O)₂R₆ₐ, and —SR₆;

R₄ₐ and R_{4b} are optionally bound together to form an aryl, heteroaryl, or heteroalicyclyl;
R₅ is selected from the group consisting of hydrogen, alkyl, alkenyl, cycloalkyl, cycloalkenyl, halogen, —CN, —SR₆, sulfonyl, —C(═O)NR₆R₆ₐ, —C(═O)R₆, —NR₆R₆ₐ, —COOR₆, and perhaloalkyl;

Z is oxygen or sulfur; and

R₆, R₆ₐ, and R_{6b} are separately selected from the group consisting of hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, and substituted or unsubstituted heteroalicyclyl.

In some embodiments, the agonist can comprise a spiro indene-indene agonist. Examples of such agonists are described, for example, in International Publication No. WO 2002/091993 to Blizzard et al., which is hereby incorporated by reference in its entirety.

In some embodiments, the agonist can be defined by Formula IX below

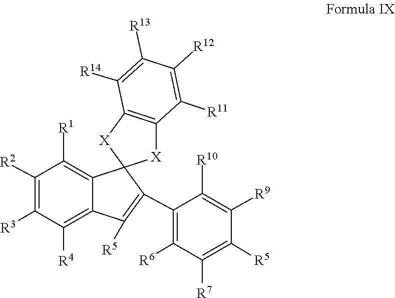

wherein

each X is independently selected from the group consisting of CH₂, C═O, C═CH₂, C═NOR^{a}, CHCH₃, CHF, CHOH, C(CH₃)OH, CF₂ and S;

R¹, R², R³, R⁴, R⁶, R⁷, R⁸, R⁹ and R¹⁰ are each independently selected from the group consisting of R^{a}, OR^{a}, OCO₂R^{a}, NR^{a}R^{a}, CO₂R^{a}, CN, Cl, F and Br;

R¹¹, R¹², R¹³ and R¹⁴ are each independently selected from the group consisting of H, R^{b}, OR^{b}, OCO₂R^{b}, NR^{a}R^{b}, CO₂R^{b}, F, Cl, CN, Br;

R⁵ is selected from the group consisting of H, F and C₁-C₆ alkyl;

R^{a} is selected from the group consisting of H, C₁-C₆ alkyl and C₁-C₆ acyl;

R^{b} is selected from the group consisting of C₂-C₇ alkyl and C₂-C₇ acyl, wherein said alkyl and acyl groups may be optionally substituted with an R^{c} group;

R^{c} is selected from the group consisting of OR^{d} and NR^{d}R^{e},

R^{d} and R^{e} are each independently selected from the group consisting of H and C₁-C₇ alkyl;

or R^{d} and R^{e} can be taken together with the nitrogen atom to which they are attached to form a 4-8 membered ring, wherein said ring is optionally interrupted by one of O, NH, NCH₃ and S and is optionally substituted with one, two, three or four C₁-C₂ alkyl groups, or one or two R^{f} groups;

R_{f} is selected from the group consisting of CH₂OH and CH₂CH₂OH;

or a pharmaceutically acceptable salt or stereoisomer thereof.

In some embodiments, the agonist can be a phenyl bicyclic agonist, such as an indenone agonist, an indene agonist, a benzofuran agonist, a benzimidazole agonist, a benzthiazole agonist, a benzoxazole agonist, a benzisoxazole agonist, an indazole agonist, an indole agonist, or a benzisothiazole agonist.

In some embodiments, the agonist can comprise an indenone agonist. Examples of such agonists are described, for example, in U.S. Pat. No. 6,903,238 to McDevitt et al., which is hereby incorporated by reference in its entirety.

In some embodiments, the agonist can be defined by Formula X below

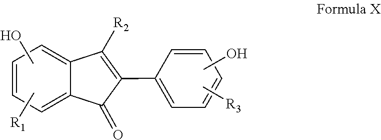

wherein

R₁ is hydrogen, hydroxyl, halogen, trifluoroalkyl of 1-6 carbon atoms, alkyl of 1-6 carbon atoms, cycloalkyl of 3-8 carbon atoms, alkenyl of 2-7 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, —CN, —NO₂, —CHFCN, —CF₂CN, aryl of 6-10 carbon atoms, —NR₄R₅, —NCOR₄, —SR₄, —SOR₄, —SO₂R₄, or a 5 or 6-membered heterocyclic ring having 1 to 4 heteroatoms selected from O, N or S; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₄, —CO₂R₄, —CONR₄R₅, or NR₄R₅;

R₂ is hydrogen, hydroxyl, alkyl of 1-6 carbon atoms, halogen, phenyl substituted with R₁, alkylthio of 1-6 carbon atoms, thioalkyl of 1-6 carbon atoms, amino, aminoalkyl of 1-6 carbon atoms, alkylamino of 1-6 carbon atoms, alkoxy of 1-6 carbon atoms, or alkenyl of 2-7 carbon atoms;

R₃ is hydrogen, halogen, hydroxyl, alkyl of 1-6 carbon atoms, alkoxy of 1-6 carbon atoms, trifluoroalkyl of 1-6 carbon atoms, or trifluoroalkoxy of 1-6 carbon atoms;

R₄ and R₅ are each, independently, hydrogen, alkyl of 1-6 carbon atoms, or aryl of 6-10 carbon atoms;

or a pharmaceutically acceptable salt thereof.

In certain embodiments, the agonist can be

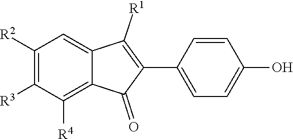

wherein R¹ is halogen (e.g., Br), R² is hydrogen, R³ is hydroxy, and R⁴ is hydrogen.

In certain embodiments, the agonist can be

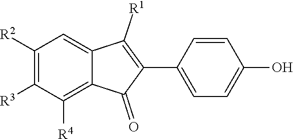

wherein R¹ is halogen (e.g., Br), R² is hydroxy, R³ is hydrogen, and R⁴ is hydrogen.

In certain embodiments, the agonist can be

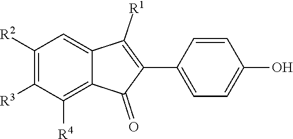

wherein R¹ is methyl, R² is hydroxy, R³ is hydrogen, and R⁴ is hydroxy.

In some embodiments, the agonist can comprise an indene agonist. Examples of such agonists are described, for example, in International Publication No. WO 2008/043567 to Jernstedt et al. and U.S. Patent Application Publication No. 2009/0326018 to Jernstedt et al., each of which is hereby incorporated by reference in its entirety.

In some embodiments, the agonist can be defined by Formula XI below

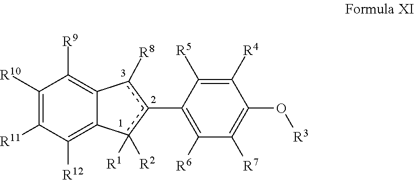

wherein either the bond between the C1 and C2 carbon atoms is a double bond or the bond between the C2 and C3 carbon atoms is a double bond, R² being absent when the bond between the C1 and C2 carbon atoms is a double bond;

R¹ and R² are independently selected from the group consisting of hydrogen, OR^{A}, C₁-C₆ alkyl, C₂-C₆ alkenyl, C₂-C₆ alkynyl, C₃-C₈ cycloalkyl, C₃-C₈ cycloalkyl C₁-C₆ alkyl, C₆-C₁₀ aryl, C₆-C₁₀ aryl C₁-C₆ alkyl, halogen, halo C₁-C₆ alkyl, dihalo C₁-C₆ alkyl and trihalo C₁-C₆ alkyl; or R¹ and R² taken together with the carbon atom to which they are attached form a double bond portion of C₂-C₆ alkenyl group;

R^{A} is selected from the group consisting of hydrogen, C₁-C₆ alkyl, C₂-C₆ alkenyl, C₂-C₆ alkynyl, C₃-C₈ cycloalkyl, C₃-C₈ cycloalkyl C₁-C₆ alkyl, C₆-C₁₀ aryl and C₆-C₁₀ aryl C₁-C₆ alkyl;

R³ is selected from the group consisting of hydrogen, C₁-C₆ alkyl, C₃-C₈ cycloalkyl and —C(O)C₁-C₄ alkyl;

R⁴, R⁵, R⁶ and R⁷ are the same or are different and each is selected from the group consisting of hydrogen, OR^{A}, halogen, cyano, nitro, C₁-C₆ alkyl, C₂-C₆ alkenyl, C₂-C₆ alkynyl, halo C₁-C₆ alkyl, dihalo C₁-C₆ alkyl and trihalo C₁-C₆ alkyl;

R⁸ is selected from the group consisting of C₃-C₈ cycloalkyl, C₃-C₈ cycloalkyl C₁-C₆ alkyl, phenyl, benzyl and C₅-C₁₀ heterocyclyl wherein said phenyl, benzyl or C₅-C₁₀ heterocyclyl group can either be unsubstituted or substituted with 1-3 substituents and each substituent is selected from the group consisting of OR^{A}, halogen, cyano, nitro, C₁-C₆ alkyl, C₂-C₆ alkenyl, C₂-C₆ alkynyl, halo C₁-C₄ alkyl, dihalo C₁-C₆ alkyl, trihalo C₁-C₆ alkyl and C(O)C₁-C₆ alkyl;

R¹⁰ is OR^{A}; and

R⁹, R¹¹ and R¹² are the same or are different and each is selected from the group consisting of hydrogen, OR^{A}, halogen, cyano, nitro, C₁-C₆ alkyl, C₂-C₆ alkenyl, C₂-C₆ alkynyl, C(O)H, C(O)C₁-C₆ alkyl, halo C₁-C₆ alkyl, dihalo C₁-C₆ alkyl and trihalo C₁-C₆ alkyl;

or a pharmaceutically acceptable ester, amide, solvate or salt thereof.

In certain embodiments, the agonist can be the compound shown below.

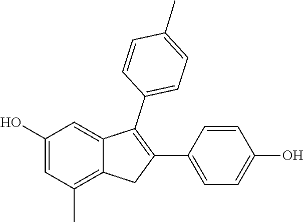 [SMILES: Cc1ccc(C2=C(c3ccc(O)cc3)Cc3c(C)cc(O)cc32)cc1]

In some embodiments, the agonist can comprise a benzofuran agonist. Examples of such agonists are described, for example, in U.S. Pat. No. 6,774,248 to Miller et al., which is hereby incorporated by reference in its entirety.

In some embodiments, the agonist can be defined by Formula XII below

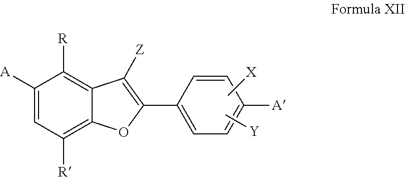

wherein

A is alkyl of 1-6 carbon atoms, halogen, trifluoroalkyl of 1-6 carbon atoms, hydroxyalkyl of 1-6 carbon atoms, —CO₂H, —NH₂, or —OP;

A′ is —OP, —CO₂P, halogen, or hydroxyalkyl;

P is hydrogen, alkyl of 1-6 carbon atoms, or phenyl;

Z is hydrogen, alkyl of 1-6 carbon atoms, halogen, —NO₂, —CN, triflouroalkyl of 1-6 carbon atoms, —COP, —CO₂P, or —C(P)═N—OP;

R and R′ are each, independently, hydrogen, alkyl of 1-6 carbon atoms, alkenyl of 2-7 carbon atoms, halogen, —OP, —SP, —SOP, —SO₂P, —SCN, trifluoroalkyl of 1-6 carbon atoms, —CF₂CF₃, trifluoroalkoxy of 1-6 carbon atoms, —NO₂, —NH₂, —NHOP, hydroxyalkyl of 1-6 carbon atoms, alkoxyalkyl of 1-6 carbon atoms per alkyl group, -alkyl-SP, -alkyl-SOP, -alkyl-SO₂P, —CN, -alkyl-CN, -alkenyl-CN, -alkylSCN, —CHFCN, —CF₂CN, -alkenyl-NO₂, haloalkyl of 1-6 carbon atoms, dihaloalkenyl of 2-7 carbon atoms, —COP, —COCF₃, —CO₂P, —CONR₁R₂, -alkyl-CONR₁R₂, -alkenyl-CON R₁R₂, -alkyl-COP, -alkenyl-COP, -alkenyl-CO₂P, -alkenyl-CO₂P, oxadiazolyl, furyl, thienyl, pyrrolyl, imidazolyl, triazolyl, or tetrazolyl;

X and Y are each, independently, hydrogen, alkyl of 1-6 carbon atoms, halogen, —NO₂, —CN, trifluoroalkyl of 1-6 carbon atoms, —OP, hydroxyalkyl of 1-6 carbon atoms, —CO₂H, or phenyl which is optionally mono- or di-substituted with hydroxyl, benzyloxy, alkoxy of 1-6 carbon atoms, or —OCH₂CH₂NR₁R₂;

R₁ and R₂ are each, independently, hydrogen, alkyl of 1-6 carbon atoms, or alkoxy of 1-6 carbon atoms; or R₁ and R₂ are concatenated together as —(CH₂)ₚ—; p=2-6;

or a pharmaceutically acceptable salt thereof.

In one example, the agonist can be the compound shown below.

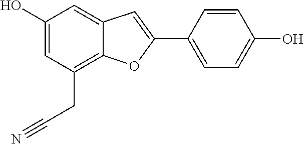 [SMILES: N#CCc1cc(O)cc2cc(-c3ccc(O)cc3)oc12]

In some embodiments, the agonist can comprise a benzimidazole agonist, a benzthiazole agonist, or a benzoxazole agonist. Examples of such agonists are described, for example, in International Publication No. WO 2002/046168 to Barlaam et al., International Publication No. WO 2002/051821 to Barlaam et al., and International Publication No. WO 2003/045930 to Bernstein, each of which is hereby incorporated by reference in its entirety.

In some embodiments, the agonist can be defined by Formula XIII below

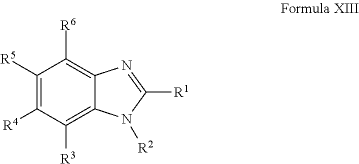

wherein

R¹ is C₁-C₈ alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁-C₈ alkyl, phenyl, benzyl or heterocycle is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; and wherein the phenyl, benzyl or heterocycle is additionally substituted by 0, 1 or 2 substituents selected from C₁-C₆ alkyl, phenyl or benzyl;

R² is H, C₁-C₆ alkyl, —(CH₂)ₘ phenyl, —(CH₂)ₘ naphthyl or —(CH₂)ₘ heterocycle, wherein the heterocycle is a 5- or 6-membered ring heterocycle containing 1, 2 or 3 heteroatoms each independently selected from O, N and S and additionally having 0 or 1 oxo groups and 0 or 1 fused benzo rings, wherein the C₁-C₆ alkyl, —(CH₂)ₘ phenyl, —(CH₂)ₘ naphthyl or —(CH₂)ₘ heterocycle are substituted with 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl;

R³ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R³ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R⁴ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁-C₃ haloalkyl;

R⁵ is —Re, —OR⁸, —SR⁸, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁-C₃ haloalkyl;

R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R⁶ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R^{a} is H, C₁-C₆ alkyl, C₁-C₃ haloalkyl, phenyl or benzyl; and

m is 0, 1, 2 or 3;

or a pharmaceutically acceptable salt or ester thereof.

In some embodiments, the agonist can be defined by Formula XIV below

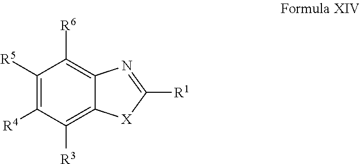

wherein:

X is O or S;

R¹ is C₁-C₈ alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁-C₈ alkyl, phenyl, benzyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl;

R³ is —R^{a}, —OR^{a}, —SR^{a}, —NR^{a}R^{a}, —CO₂R^{a}, —OC(═O)R^{a}, —C(═O)NR^{a}R^{a}, —NR²C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R³ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R⁴ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁-C₃ haloalkyl;

R⁵ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁-C₃ haloalkyl;

R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R⁶ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro; and

R^{a} is H, C₁-C₆ alkyl, C₁-C₃ haloalkyl, phenyl or benzyl; or

a pharmaceutically acceptable salt or ester thereof.

In some embodiments, the agonist can be defined by Formula XV below

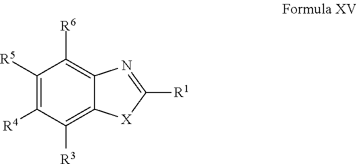

wherein:

X is O or S;

R¹ is C₁-C₈ alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁-C₈ alkyl, phenyl, benzyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl;

R³ is —Re, —OR^{a}, —SR, —NR^{a}R^{a}, —CO₂R^{a}, —OC(═O)R^{a}, —C(═O)NR^{a}R^{a}, —NR^{a}C(O)R^{a}—NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R³ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R⁴ is H or —NR^{a}R^{b};

R⁵ is H or —NR^{a}R^{b}; wherein R⁴ and R⁵ are not both H;

R⁶ is —R^{a}, —OR^{a}, —SR^{a}, —NR^{a}R^{a}, —CO₂R^{a}, —OC(═O)R^{a}, —C(═O)NR^{a}R^{a}, NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁-C₃ haloalkyl; or R⁶ is C₁-C₃ alkyl containing 1 or 2 substituents selected from —OR^{a}, —SR^{a}, —NR^{a}R^{a}, —CO₂R^{a}, —OC(═O)R^{a}, —C(═O)NR^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R^{a} is H, C₁-C₆ alkyl, C₁-C₃ haloalkyl, phenyl or benzyl; and

R^{b} is C₁-C₈ alkyl, C₁-C₈ alkyl C₄-C₈ cycloalkyl, C₂-C₆ alkenyl, C₂-C₆ alkenyl-Ph, C₂-C₆ alkenyl-Het, —(CH₂)ₙ-Ph or —(CH₂)ₙ-Het wherein n is 0-4 and Het is a 5- or 6-membered ring heterocycle containing 1, 2 or 3 heteroatoms each independently selected from O, N and S and additionally having 0 or 1 oxo groups and 0 or 1 fused benzo rings, wherein the C₁-C₄ alkyl, phenyl or heterocycle is substituted by 0, 1, 2 or 3 substituents selected from —Re, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, NR^{a}S(═O)₂R^{a}—C(═O)R^{a}, —S(═O)R^{a}, —S(═O)₂R^{a}, halogen, cyano, nitro and C₁-C₃ haloalkyl;

or a pharmaceutically acceptable salt or ester thereof.

In certain embodiments, the agonist can be the compound shown below.

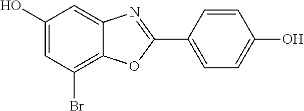 [SMILES: Oc1ccc(-c2nc3cc(O)cc(Br)c3o2)cc1]

In some embodiments, the agonist can comprise a naphthyl-benzoxazole agonist or a benzoxazole agonist. Examples of such agonists are described, for example, in U.S. Pat. No. 6,960,607 to Malamas et al. and U.S. Pat. No. 6,794,403 to Malamas et al., each of which is hereby incorporated by reference in its entirety.

In some embodiments, the agonist can be defined by Formula XVI below

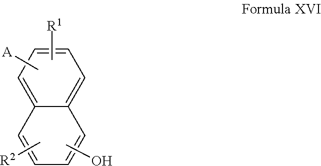

wherein

A is one of the groups shown below

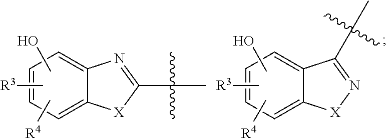

R¹, R³, and R⁴ are each, independently, hydrogen, hydroxyl, halogen, alkyl of 1-6 carbon atoms, cycloalkyl of 3-8 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR⁵, —CO₂R⁵, —NO₂, CONR⁵R⁶, NR⁵R⁶ or N(R⁵)COR⁶;

R² is hydrogen, hydroxyl, halogen, alkyl of 1-6 carbon atoms, triflouroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R⁵ and R⁶ are each, independently, hydrogen, alkyl of 1-6 carbon atoms, alkenyl of 2-7 carbon atoms, aryl of 6-10 carbon atoms, —CN, —CHFCN, or —CF₂CN; wherein the alkyl or alkenyl moieties are optionally substituted with hydroxyl, —CN, halogen, trifluoroalkyl of 1-6 carbon atoms, trifluoroalkoxy of 1-6 carbon atoms, —COR⁷, —CO₂R⁷, —NO₂, CONR⁷R⁸, NR⁷R⁸ or N(R⁷)COR⁸;

R⁷ and R⁸ are each, independently, hydrogen, alkyl of 1-6 carbon atoms, or aryl of 6-10 carbon atoms;

X is O, S, or NR⁹;

R⁹ is hydrogen, alkyl of 1-6 carbon atoms, aryl of 6-10 carbon atoms, COR⁵, CO₂R⁵, or SO₂R⁵;

or a pharmaceutically acceptable salt or ester thereof.

In some embodiments, the agonist can be defined by Formula XVII below

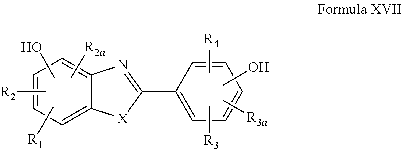

wherein

R₁ is hydrogen, hydroxyl, halogen, alkyl of 1-6 carbon atoms, triflouroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or —SO₂R₅;

or a pharmaceutically acceptable salt or ester thereof.

In certain embodiments, the agonist can be one of the compounds shown below.

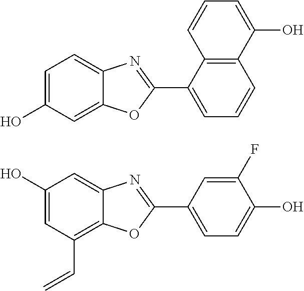

In some embodiments, the agonist can comprise a benzimidazole agonist. Examples of such agonists are described, for example, in U.S. Patent Application Publication No. 2004/0002524 to Chesworth et al., which is hereby incorporated by reference in its entirety.

In some embodiments, the agonist can be defined by Formula XVIII below

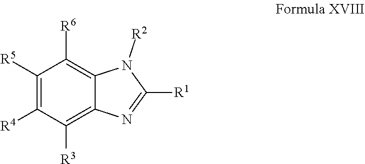

or a pharmaceutically acceptable salt or ester thereof, wherein

R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl, wherein the (C₁-C₆)alkyl; phenyl; (C₂-C₆)heteroaryl; (C₃-C₇)cycloalkyl; or (C₄-C₈)cycloalkenyl groups of R¹ or R² are optionally substituted by from 1 to 3 substituents independently selected from the group consisting of halogen; (C₁-C₆)alkyl; (C₃-C₈)cycloalkyl; (C₄-C₈)cycloalkenyl; (C₁-C₆)alkoxy; hydroxy; R¹² CO₂, R¹²R¹³NCO, R¹²R¹³N; (C₁-C₆)alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)alkoxy; R¹²R¹³N(C₁-C₆ alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of halogen, (C₁-C₆)alkyl; (C₃-C₈)cycloalkyl; (C₄-C₈)cycloalkenyl; (C₁-C₆)alkoxy, hydroxy, R¹² CO₂, R¹²R¹³NCO, R¹²R¹³N; (C₁-C₆)alkylcarbonyl, —CHO, cyano, thio; R¹²SO₂(C₁-C₆)alkyl; R¹² CO₂(C₁-C₆)alkyl; R¹²R¹³NCO(C₁-C₆)alkyl; R¹ CO(C₁-C₆)alkyl; R¹² SO₂(C₁-C₆)alkoxy; R¹² CO₂(C₁-C₆)alkoxy; R¹²R¹³NCO(C₁-C₆)alkoxy; R¹²CO(C₁-C₆)alkoxy; R¹²R¹³N SO₂(C₁-C₆)alkyl; and R¹²R¹³N SO₂(C₁-C₆) alkoxy; wherein R¹² and R¹³ are each independently selected from the group consisting of hydrogen; (C₁-C₇)alkyl; (C₃-C₈)cycloalkyl; (C₄-C₈)cycloalkenyl; (C₆-C₁₀) aryl; (C₂-C₁₀)alkenyl, (C₂-C₁₀)alkynyl; (C₂-C₄)heteroaryl; (C₁-C₆)alkylaryl; (C₁-C₆) alkyl(C₂-C₆)heteroaryl; (C₂-C₆)alkoxyaryl; (C₂-C₆) alkoxy(C₂-C₆)heteroaryl; or R¹² and R¹³ taken together form a three to eight membered heterocyclic ring having 1 to 3 heteroatoms; n is from 0 to 5; and x is 1 or 2;

or R¹ and R² are each independently a group of the Formula A:

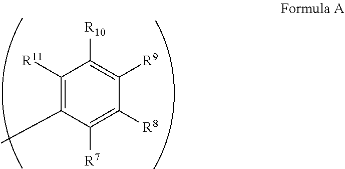

wherein R⁷, R⁸, R¹⁰ and R¹¹ are each independently hydrogen; hydroxy; (C₁-C₆) alkyl; (C₁-C₆)alkoxy; or halogen;

R⁹ is hydroxy; (C₁-C₆) alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy; and

R³, R⁴, R⁵ and R⁶ are each independently hydrogen, hydroxy; (C₁-C₆)alkyl; (C₁-C₆)alkoxy, or halogen;

with the proviso that at least one of R¹ or R² must be the group of Formula A.

In some embodiments, the agonist can be one of the compounds below.

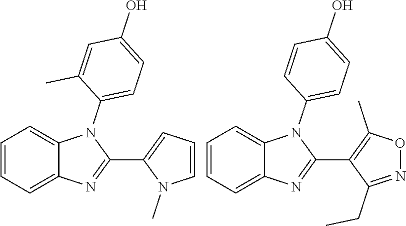

In some embodiments, the agonist can be an indazole agonist, such as the compound shown below.

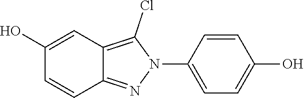 [SMILES: Oc1ccc(-n2nc3ccc(O)cc3c2Cl)cc1]

In some embodiments, the agonist can comprise an indole agonist. Examples of such agonists are described, for example, in Japanese Patent Application Publication No. JP2001122855 to Fujii et al., and U.S. Patent Application Publication No. 2003/0220377 to Chesworth, each of which is hereby incorporated by reference in its entirety.

In some embodiments, the agonist can be defined by Formula XIX below

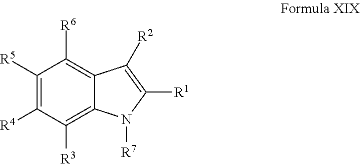

or a pharmaceutically acceptable salt or ester thereof, wherein

R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl; wherein the (C₁-C₆)alkyl; phenyl; (C₂-C₆)heteroaryl; (C₃-C₈)cycloalkyl; or (C₄-C₈) cycloalkenyl groups of R¹ or R² are optionally substituted by from 1 to 3 substituents independently selected from the group consisting of halogen; (C₁-C₆)alkyl; (C₃-C₈)cycloalkyl; (C₄-C₅)cycloalkenyl; (C₁-C₆)alkoxy; hydroxy; R¹²CO², R¹²R¹³NCO, R¹²R¹³N; (C₁-C₆) alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)alkoxy; R¹²R¹³N(C₁-C₆)alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of halogen, (C₁-C₆)alkyl; (C₃-C₈)cycloalkyl; (C₄-C₈)cycloalkenyl; (C₁-C₆)alkoxy, hydroxy, R¹²CO₂, R¹²R¹³NCO, R¹²R¹³N; (C₁-C₆)alkylcarbonyl, —CHO, cyano, thio; R¹²SO₂(C₁-C₆)alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆)alkoxy; R¹²CO(C₁-C₆)alkoxy; R¹²R¹³N SO₂(C₁-C₆)alkyl; and R¹²R¹³N SO₂(C₁-C₆) alkoxy, wherein R¹² and R¹³ are each independently selected from the group consisting of hydrogen; halogen; (C₁-C₇)alkyl; (C₃-C₈)cycloalkyl; (C₄-C₈)cycloalkenyl; (C₆-C₁₀) aryl; (C₂-C₁₀)alkenyl, (C₂-C₁₀)alkynyl; (C₂-C₄)heteroaryl; (C₁-C₆)alkylaryl; (C₁-C₆)alkyl (C₂-C₆)heteroaryl; (C₂-C₆)alkoxyaryl; (C₂-C₆) alkoxy(C₂-C₆)heteroaryl; or R¹² and R¹³ taken together form a three to eight membered heterocyclic ring having up to 3 heteroatoms; n is from 0 to 5; and x is 1 or 2;

or R¹ and R² are each independently a group of the Formula A

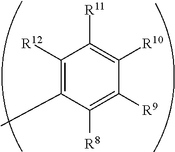

wherein R⁸, R⁹, R¹¹ and R¹² are each independently hydrogen; hydroxy; (C₁-C₆)alkyl; (C₁-C₆)alkoxy; or halogen;

R¹⁰ is hydrogen; hydroxy; (C₁-C₆)alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy;

R³, R⁴, R⁵ and R⁶ are each independently hydrogen, hydroxy; (C₁-C₆)alkyl; (C₁-C₆)alkoxy; or halogen; and

R⁷ is H or (C₁-C₃)alkyl;

with the proviso that at least one of R¹ or R² must be the group of Formula A.

In certain embodiments, the agonist can be a compound shown below.

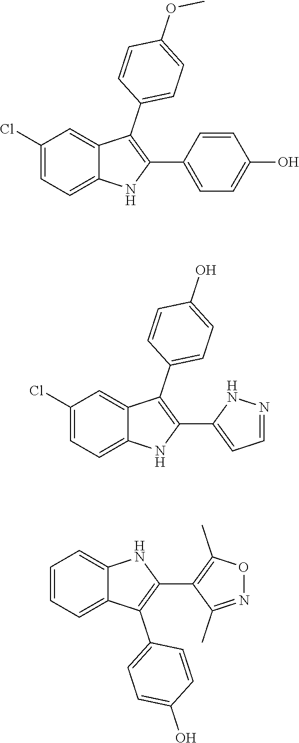 [SMILES: COc1ccc(-c2c(-c3ccc(O)cc3)[nH]c3ccc(Cl)cc23)cc1 ; Oc1ccc(-c2c(-c3ccn[nH]3)[nH]c3ccc(Cl)cc23)cc1 ; Cc1noc(C)c1-c1[nH]c2ccccc2c1-c1ccc(O)cc1]

In some embodiments, the agonist can comprise an indazole agonist, a benzisoxazole agonist, or a benzisothiazole agonist. Examples of such agonists are described, for example, in International Publication No. WO 2006/040351 to Rondot et al., which is hereby incorporated by reference in its entirety.

In some embodiments, the agonist can be defined by Formula XX below

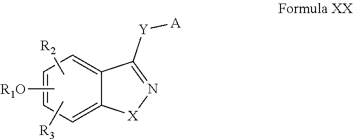

or a pharmaceutically acceptable salt or ester thereof, wherein:

R₁ is hydrogen or a (C₁-C₆)alkyl, (C₃-C₆)cycloalkyl, trifluoromethyl, —N═CR₅R₆, —SO₂NR₇R₈, phenyl, phenyl(C₁-C₃)alkyl or (C₁-C₃)alkyl substituted by a saturated heterocyclic radical, wherein the phenyl is unsubstituted or substituted by at least one substituent selected from the group consisting of a hydroxyl, a halogen, a nitro, a cyano, a (C₁-C₃)alkyl, a (C₁-C₃)alkoxy and a trifluoromethyl; R₁ can also be a salt;

R₂ and R₃ are each independently hydrogen or a hydroxyl, halogen, nitro, cyano, (C₁-C₆)alkyl, (C₃-C₆)cycloalkyl, (C₁-C₆)alkoxy, trifluoromethyl, —NR₇R₈, —CONR₇R₈, —COR₉ or —CO₂R₉ group; R² can also be a phenyl or a saturated or unsaturated heterocycle, wherein the phenyl is unsubstituted or substituted by at least one substituent selected from the group consisting of a hydroxyl, a halogen, a nitro, a cyano, a (C₁-C₃)alkyl, a (C₁-C₃)alkoxy, a trifluoromethyl and a saturated heterocyclic radical;

X is O, S, SO, SO₂ or NR₄;

R₄ is hydrogen or a (C₁-C₆)alkyl, (C₃-C₆)cycloalkyl, phenyl, phenyl(C₁-C₃)alkyl, (C₁-C₃)alkyl substituted by a saturated heterocyclic radical, —COR₇, —CO₂R₇ or —SO₂NR₇R₈ group, wherein the phenyl is unsubstituted or substituted by at least one substituent selected from the group consisting of a hydroxyl, a halogen, a nitro, a cyano, a (C₁-C₃)alkyl, a (C₁-C₃)alkoxy, a trifluoromethyl, a phenyl(C₁-C₃)alkyl and a phenyl(C₁-C₃)alkoxy;

Y is direct bond, O, S, SO, SO₂, NR₄, CO, —(CR₁₀R₁₁)ₙ— or —R₁₀C═CR₁₁—;

R₅, R₆, R₇ and R₈ are each independently hydrogen or a (C₁-C₆)alkyl or (C₃-C₆)cycloalkyl group;

R₉ is hydrogen, a (C₁-C₆)alkyl, a phenyl or a saturated or unsaturated heterocyclic radical, wherein the phenyl is unsubstituted or substituted by at least one substituent selected from the group consisting of a hydroxyl, a halogen, a nitro, a cyano, a (C₁-C₃)alkyl, a (C₁-C₃)alkoxy, a trifluoromethyl and a saturated heterocyclic radical;

R₁₀ and R₁₁ are each independently hydrogen or a cyano, (C₁-C₆)alkyl, —CO-phenyl, —CO (unsaturated heterocyclic radical) or —CONR₇R₈ group, wherein the phenyl is unsubstituted or substituted by at least one substituent selected from the group consisting of a hydroxyl, a halogen, a nitro, a cyano, a (C₁-C₃)alkyl, a (C₁-C₃)alkoxy and a trifluoromethyl;

n is 1 or 2; and

A is a (C₃-C₁₅)cycloalkyl, a (C₃-C₁₅)cycloalkene, a phenyl or a naphthyl, wherein the cycloalkyl or the cycloalkene is unsubstituted or substituted by at least one (C₁-C₆)alkyl, and wherein the phenyl or the naphthyl is unsubstituted or substituted by at least one substituent selected from the group consisting of a hydroxyl, a halogen, a nitro, a cyano, a (C₁-C₃)alkyl, a (C₁-C₃)alkoxy and a trifluoromethyl;

wherein when X is NR₄, Y and R₂ together with the indazole ring bearing them can also form a 1H-pyrano[4,3,2-cd]indazole.

In one embodiment, the agonist can be a compound shown below.

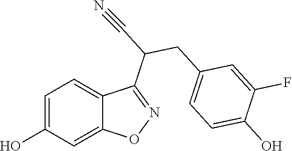 [SMILES: N#CC(Cc1ccc(O)c(F)c1)c1noc2cc(O)ccc12]

In some embodiments, the agonist can comprise a bicyclic benzopyran agonist. Examples of such agonists are described, for example, in International Publication No. WO 2003/074044 to Lugar et al., which is hereby incorporated by reference in its entirety.

In some embodiments, the agonist can be defined by Formula XXI below

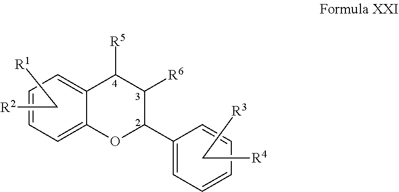

wherein

R¹, R², R³, and R⁴, are each independently hydrogen, substituted or unsubstituted alkyl, hydroxy, substituted or unsubstituted alkoxy, halogen, or —CF₃; and

R⁵ and R⁶ are each independently hydrogen or substituted or unsubstituted alkyl;

or a pharmaceutically salt thereof.

In certain embodiments, the agonist can be one of the compounds shown below.

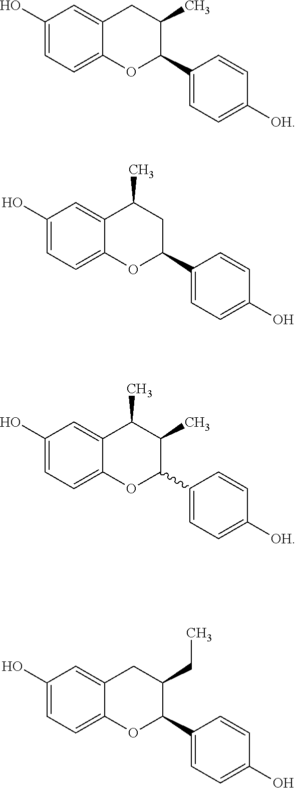 [SMILES: C[C@@H]1Cc2cc(O)ccc2O[C@@H]1c1ccc(O)cc1 ; C[C@H]1C[C@@H](c2ccc(O)cc2)Oc2ccc(O)cc21 ; C[C@@H]1c2cc(O)ccc2OC(c2ccc(O)cc2)[C@@H]1C ; CC[C@@H]1Cc2cc(O)ccc2O[C@@H]1c1ccc(O)cc1]

In some embodiments, the agonist can comprise a 3-alkyl-4-benzylchromane agonist. Examples of such agonists are described, for example, in U.S. Patent Application Publication No. 2003/0069303 to Veeneman et al., which is hereby incorporated by reference in its entirety.

In some embodiments, the agonist can be defined by Formula XXII below

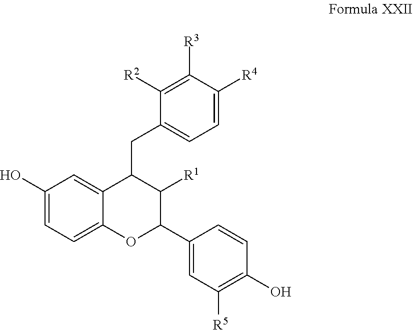

or a pharmaceutically acceptable salt or ester thereof, wherein

R¹ is (1C-4C)alkyl, (2C-4C)alkenyl or (2C-4C)alkynyl, and independently R¹ has a cis-orientation in relation to the exocyclic phenyl group at the 2-position of the skeleton;

R⁴ is halogen, —CF₃, OH or (1C-2C)alkyloxy; and

R², R³, and Rare independently H, halogen, —CF₃, (1C-4C)alkyl, (2C-4C)alkenyl, or (2C-4C)alkynyl.

In some embodiments, the agonist can comprise a compound described in International Publication No. WO 2009/002802 to Cohen, which is hereby incorporated by reference in its entirety.

In some embodiments, the agonist can be defined by Formula XXIII below

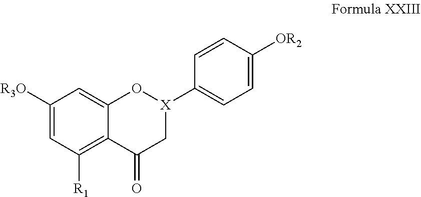

wherein

X is an asymmetric carbon atom having an S or R configuration;

R₁ is selected from the group consisting of H and OR₄; and

R₂, R₃, and R₄ are independently selected from the group consisting of H, and glycoside, glucuronide, acyl, phosphate, phosphonic acid, alkyl phosphonate, sulfate, C₁ to C₆ alkyl, C₃ to C₆ cycloalkyl, aryl, carbonate, and carbamate; each optionally substituted with from one to three groups selected from hydrogen, C₁ to C₆ alkyl, phenyl, benzyl, alkylphenyl, hydroxy, alkoxy, acyloxy, amino, carboxy and alkoxycarbonyl;

or a pharmaceutically acceptable salt, or prodrug thereof.

In certain embodiments, the agonist can be the compound defined below.

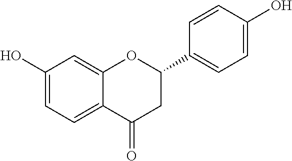 [SMILES: O=C1C[C@@H](c2ccc(O)cc2)Oc2cc(O)ccc21]

In some embodiments, the agonist can comprise a compound described in International Publication No. WO 2007/053915 to Keukeleire et al., which is hereby incorporated by reference in its entirety.

In some embodiments, the agonist can be defined by Formula XXIV below

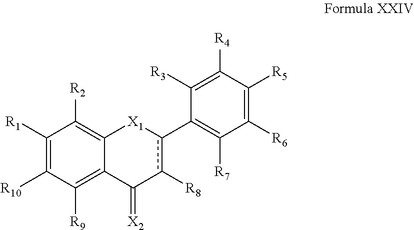

or any stereoisomer, tautomer, solvate or pharmaceutically acceptable salt thereof, wherein

the dotted line represents optionally a double bond;

R₁, R₈, R₉, and R₁₀ are independently selected from OH, H, halogen, cyano, amino, nitro, nitroso, C₁-C₅ alkyl, C₁-C₅ alkoxy, C₁-C₈ alkylcarbonyloxy; wherein each of said alkyl, alkoxy or alkylcarbonyloxy group is linear or branched;

R₂ is selected from a branched C₅ alkyl, a C₆-C₂₀ alkyl, C₆-C₂₀ alkenyl, C₆-C₂₀ alkynyl or C₆-C₂₀ alkenynyl, wherein each of said C₆-C₂₀ alkyl, alkenyl, alkynyl or alkenynyl group is linear or branched;

R₃, R₄, R₅, R₆, and R₇ are independently selected from OH, H, halogen, cyano, amino, nitro or nitroso;

X₁ is selected from O, S;

X₂ is selected from O, S or the two bonds are each separately formed with a hydrogen atom, wherein said naringenin derivative is not 8-geranylnaringenin.

In some embodiments, the agonist can comprise a chroman-7-ol agonist. Examples of such agonists are described, for example, in U.S. Pat. No. 7,396,855 to Setchell et al., and U.S. Pat. No. 7,528,267 to Setchell et al., each of which is hereby incorporated by reference in its entirety.

In some embodiments, the agonist can comprise a tetrahydroisoquinoline agonist. Examples of such agonists are described, for example, in U.S. Pat. No. 6,686,351 to Bhagwat et al., and U.S. Pat. No. 7,256,201 to Barlaam et al., each of which is hereby incorporated by reference in its entirety.

In some embodiments, the agonist can comprise a quinazoline or quinazolinone agonist. Examples of such agonists are described, for example, in U.S. Pat. No. 7,381,730 and International Publication No. WO 2006/116401, each of which is hereby incorporated by reference in its entirety.

In some embodiments, the agonist can comprise a 3-arylhydroxybenzoxazine agonist. Examples of such agonists are described, for example, in U.S. Pat. No. 7,015,219, which is hereby incorporated by reference in its entirety.

In some embodiments, the agonist can comprise a 2-arylquinoline agonist. Examples of such agonists are described, for example, in U.S. Patent Application Publication No. 2005/0009784, which is hereby incorporated by reference in its entirety.

In some embodiments, the agonist can comprise a 2-phenylnaphthalene agonist. Examples of such agonists are described, for example, in U.S. Pat. No. 6,914,074, which is hereby incorporated by reference in its entirety.

In some embodiments, the agonist can comprise a phenylnaphthalene agonist. Examples of such agonists are described, for example, in U.S. Patent Application Publication No. 2007/0225330, which is hereby incorporated by reference in its entirety.

In some embodiments, the agonist can comprise an isoquinolinone agonist. Examples of such agonists are described, for example, in International Publication No. 2008/091555 to Dalton et al., which is hereby incorporated by reference in its entirety.

In some embodiments, the agonist can be defined by Formula XXV below

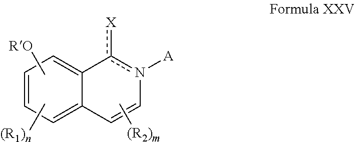

wherein

A is

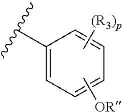

### and X is O or S;

or A is nothing, N forms a double bond with the cyclic carbon and X is OH or OCH₂CH₂-heterocycle in which the heterocycle is a 3-7 member saturated or unsaturated, substituted or unsubstituted heterocyclic ring;

R₁, R₂, and R₃ are independently hydrogen, aldehyde, COOH, —C(═NH)—OH, CHNOH, CH═CHCO₂H, CH═CHCO₂R, —CH═CH₂, hydroxyalkyl, halogen, hydroxyl, alkoxy, cyano, nitro, CF₃, NH₂, 4-Ph-OMe, 4-Ph-OH, SH, COR, COOR, OCOR, alkenyl, allyl, 2-methylallyl, alkynyl, propargyl, OSO₂CF₃, OSO₂CH₃, NHR, NHCOR, N(R)₂, sulfonamide, SO₂R, alkyl, haloalkyl, aryl, phenyl, benzyl, protected hydroxyl, OCH₂CH₂NR₄R₅, Z-Alkyl-Q, Z-Alkyl-NR₄R₅, Z-Alkyl-heterocycle or OCH₂CH₂-heterocycle in which the heterocycle is a 3-7 member saturated or unsaturated, substituted or unsubstituted heterocyclic ring;

R is alkyl, hydrogen, haloalkyl, dihaloalkyl, trihaloalkyl, CH₂F, CHF₂, CF₃, CF₂CF₃, aryl, phenyl, benzyl, -Ph-CF₃, -Ph-CH₂F, -Ph-CHF₂, -Ph-CF₂CF₃, halogen, alkenyl, CN, NO₂ or OH;

R¹ is hydrogen, alkyl, or —COR;

R″ is hydrogen, alkyl, or —COR;

R₄ and R₅ are independently hydrogen, phenyl, benzyl, an alkyl group of 1 to 6 carbon atoms, a 3 to 7 member cycloalkyl, heterocycloalkyl, aryl or heteroaryl group; Z is O, NH, CH₂ or

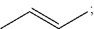

Q is SO₃H, CO₂H, CO₂R, NO₂, tetrazole, SO₂NH₂ or SO₂NHR;

n is an integer between 1-3;

m is an integer between 1-2;

p is an integer between 1-4; and

Alkyl is a linear alkyl of 1-7 carbons, branched alkyl of 1-7 carbons, or cyclic alkyl of 3-8 carbons.

In some embodiments, the agonist can comprise a benzopyran agonist or a tetralin agonist. Examples of such agonists are described, for example, in U.S. Pat. No. 6,630,508, International Publication No. WO 2004/094400, U.S. Pat. No. 7,279,499, and International Publication No. WO 2006/088716 to Krishnan et al., each of which is hereby incorporated by reference in its entirety.

In some embodiments, the agonist can be defined by Formula XXVI below

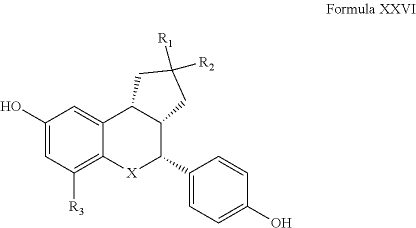

wherein

X is CH or O;

R₁, R₂, and R₃ are independent hydrogen, halogen (e.g., F), substituted or unsubstituted alkyl (e.g., substituted or unsubstituted C₁-C₆ alkyl), —CF₃, CN, or substituted or unsubstituted alkoxy (e.g., substituted or unsubstituted C₁-C₆ alkoxy).

In some embodiments, the agonist can be defined by Formula XXVII below

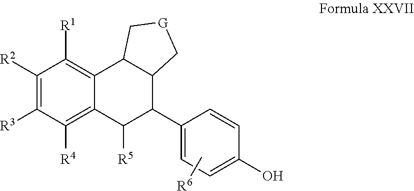

wherein

G is —CH₂—, —CH₂CH₂—, —CH₂C(CH₃)₂— or —O—;

R¹ is hydrogen, hydroxy or amino;

R² is hydrogen or hydroxy;

R³ is hydrogen, hydroxy, Br, methyl, n-propyl, i-propyl, n-butyl, hydroxymethyl, methoxy, CH₃CH(OH)—, acetyl, CH₃OCH₂—, R⁷C(O)CH₂CH₂— Or R⁸CH₂CH₂CH₂—; or R² and R³ form a —CH₂CH₂—X—O— biradical, wherein the oxygen radical represents the R² end and the methylene radical represents the R³ end;

X is —C(O)— or —C(C═CH₂)—;

R⁴ is hydrogen, hydroxy, cyano, R⁹—CH₂—, vinyl, 4-chlorophenyl, carboxy, aminocarbonyl or methoxycarbonyl;

R⁵ is hydrogen, methyl, ethyl, R¹⁰—CH₂—, CH₃CH(OH)—, acetyl, carboxyl or methoxycarbonyl;

R⁶ is hydrogen or fluoro;

R⁷ is amino, methylamino, dimethylamino or piperidin-1-yl;

R⁸ is bromo, hydroxy, dimethylamino or methoxy;

R⁹ is bromo, cyano, hydroxy, methoxy or azido;

R¹⁰ is bromo, hydroxy, cyano, methoxy or pyrrolidin-1-yl;

with the provisos that: at least one or R¹, R², R³ and R⁴ is hydroxy and at least three of R¹, R², R³, R⁴ and R⁵ are hydrogen;

and enantiomers thereof.

In some embodiments, the agonist can be defined by Formula XXVIII or Formula XXIX below

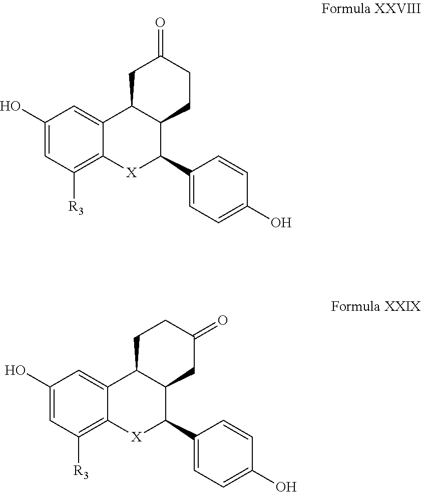

wherein

X is CH or O; and

R³ are independent hydrogen, halogen (e.g., F), substituted or unsubstituted alkyl (e.g., substituted or unsubstituted C₁-C₆ alkyl), —CF₃, CN, or substituted or unsubstituted alkoxy (e.g., substituted or unsubstituted C₁-C₆ alkoxy).

In some embodiments, the agonist can comprise a tri- or tetracyclic tetrahydrofluorene agonist. Examples of such agonists are described, for example, in U.S. Pat. Nos. 7,157,604, 7,087,599, International Publication No. WO 2002/041835, International Publication No. WO 2006/062876, International Publication No. WO 2007/089291, and U.S. Pat. No. 7,151,196, each of which is hereby incorporated by reference in its entirety.

In some embodiments, the agonist can be defined by Formula XXX below

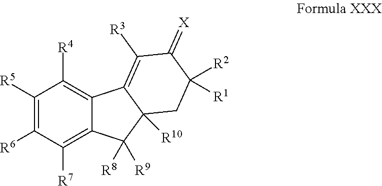

wherein

X is selected from the group consisting of: O, N—OR^{a}, N—NR^{a}R^{b} and C₁-C₆ alkylidene, wherein said alkylidene group is unsubstituted or substituted with a group selected from hydroxy, amino, O(C₁-C₄ alkyl), NH(C₁-C₄ alkyl), or N(C₁-C₄ alkyl)₂;

R¹ is selected from the group consisting of hydrogen, C₁-C₆ alkyl, C₂-C₆ alkenyl, and C₂-C₆ alkynyl, wherein said alkyl, alkenyl and alkynyl groups are either unsubstituted or substituted with a group selected from OR^{c}, SR^{c}, NR^{b}R^{c}, C(═O)R^{c}, C(═O)CH₂OH, or phenyl, wherein said phenyl group can either be unsubstituted or substituted with 1-3 substituents independently selected from the group consisting of C₁-C₄ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

R² is selected from the group consisting of hydrogen, hydroxy, iodo, O(C═O)R^{c}, C(═O)R^{c}, CO₂R^{c}, C₁-C₆ alkyl, C₂-C₆ alkenyl, and C₂-C₆ alkynyl, wherein said alkyl, alkenyl and alkynyl groups are either unsubstituted or substituted with a group selected from OR^{c}, SR^{c}, NR^{b}R^{c}, C(═O)R^{c}, C(═O)CH₂OH, or phenyl, wherein said phenyl group can either be unsubstituted or substituted with 1-3 substituents independently selected from the group consisting of C₁-C₄ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

or R¹ and R², when taken together with the carbon atom to which they are attached, form a carbonyl group;

or R¹ and R², when taken together, form a C₁-C₆ alkylidene group, wherein said alkylidene group is either unsubstituted or substituted with a group selected from the group consisting of hydroxy, O(C₁-C₄ alkyl), N(C₁-C₄ alkyl)₂, and phenyl, wherein said phenyl group can either be unsubstituted or substituted with 1-3 substituents independently selected from the group consisting of C₁-C₄ alkyl, OH, O(C₁-C₄ alkyl), NH₂, NH(C₁-C₄ alkyl), NH(C₁-C₄ alkyl)₂, halo, CN, NO₂, CO₂H, CO₂(C₁-C₄ alkyl), C(O)H, and C(O)(C₁-C₄ alkyl);

R³ is selected from the group consisting of hydrogen, fluoro, chloro, bromo, iodo, cyano, nitro, NR^{a}R^{c}, OR^{a}, C(═O)R^{a}, CO₂R^{c}, CONR^{a}R^{c}, SR^{a}, S(═O)R^{a}, SO₂R^{a}, C₁-C₁₀ alkyl, C₂-C₁₀ alkenyl, C₂-C₁₀ alkynyl, C₃-C₇ cycloalkyl, C₅-C₇ cycloalkenyl, 4-7 membered heterocycloalkyl, (cycloalkyl)alkyl, (cycloalkenyl)alkyl, (heterocycloalkyl)alkyl, aryl, heteroaryl, arylalkyl, and (heteroaryl)alkyl, wherein said alkyl, alkenyl, alkynyl, cycloalkyl, cycloalkenyl, heterocycloalkyl, aryl and heteroaryl groups are either unsubstituted or independently substituted with 1, 2 or 3 groups selected from fluoro, chloro, bromo, iodo, cyano, oxo, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, YR^{d}, and ZYR^{d};

R⁴ and R⁵ are each independently selected from the group consisting of hydrogen, hydroxy, amino, methyl, CF₃, fluoro, chloro, and bromo;

R⁶ is selected from the group consisting of NH₂, NH(C═O)R^{e}, and NH(C═O)OR^{e};

R⁷ is selected from the group consisting of hydrogen, OR^{b}, NR^{c}, fluoro, chloro, bromo, iodo, cyano, nitro, C₁-C₆ alkyl, C₂-C₆ alkenyl, CF₃, and CBF₂;

R⁸ and R⁹ are each independently selected from the group consisting of hydrogen, fluoro, chloro, C₁-C₆ alkyl, C₂-C₆ alkenyl, and C₂-C₆ alkynyl,

or R⁸ and R⁹, when taken together with the carbon atom to which they are attached, form a 3-5 membered cycloalkyl ring,

or R⁸ and R⁹, when taken together with the carbon atom to which they are attached, form a carbonyl group;

R¹⁰ is selected from the group consisting of hydrogen, C₁-C₁₀ alkyl, C₂-C₁₀ alkenyl, C₂-C₁₀ alkynyl, C₃-C₆ cycloalkyl, C₄-C₆ cycloalkenyl, (cycloalkyl)alkyl, (cycloalkyl)alkenyl, (cycloalkenyl)alkyl, aryl, heteroaryl, arylalkyl and (heteroaryl)alkyl, wherein said alkyl, alkenyl, alkynyl, cycloalkyl, cycloalkenyl, (cycloalkyl)alkyl, (cycloalkyl)alkenyl, (cycloalkenyl)alkyl, aryl, heteroaryl, arylalkyl and (heteroaryl)alkyl groups can be optionally substituted with a group selected from bromo, iodo, OR^{b}, SR^{b}, C(═O)R^{b}, 1-3 C₁-C₃ alkyl, 1-3 chloro, or 1-5 fluoro,

or R¹⁰ and R¹, when taken together with the three intervening carbon atoms to which they are attached, form a 5-6 membered cycloalkyl or cycloalkenyl ring which can be optionally substituted with 1-3 groups independently selected from oxo, hydroxy, fluoro, C₁-C₆ alkyl, C₂-C₆ alkenyl, C₂-C₆ alkynyl, C₁-C₆ alkylidenyl, C₃-C₆ cycloalkyl, (cycloalkyl)alkyl, phenyl, or phenylalkyl, wherein said alkyl, alkenyl, alkynyl, alkylidenyl, cycloalkyl, (cycloalkyl)alkyl, phenyl, and phenylalkyl groups can be optionally substituted with a group selected from chloro, bromo, iodo, OR^{b}, SR^{b}, C₁-C₃ alkyl, C(═O)R^{b}, or 1-5 fluoro;

R^{a} is selected from the group consisting of hydrogen, C₁-C₁₀ alkyl, and phenyl, wherein said alkyl group can be optionally substituted with a group selected from hydroxy, amino, O(C₁-C₄ alkyl), NH(C₁-C₄ alkyl), N(C₁-C₄ alkyl)₂, phenyl, or 1-5 fluoro, and wherein said phenyl groups can either be unsubstituted or substituted with 1-3 substituents independently selected from the group consisting of C₁-C₄ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

R^{b} is selected from the group consisting of hydrogen, C₁-C₁₀ alkyl, benzyl and phenyl, wherein said phenyl group can either be unsubstituted or substituted with 1-3 substituents independently selected from the group consisting of C₁-C₄ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

R^{c} is selected from the group consisting of hydrogen, C₁-C₁₀ alkyl and phenyl, wherein said phenyl group can either be unsubstituted or substituted with 1-3 substituents independently selected from the group consisting of C₁-C₄ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

or R^{a} and R^{c}, whether or not on the same atom, can be taken together with any attached and intervening atoms to form a 4-7 membered ring;

R^{d} is selected from the group consisting of NR^{b}R^{c}, OR^{a}, CO₂R^{a}, O(C═O)R^{a}, CN, NR^{c}(C═O)R^{b}, CONR^{a}R^{c}, SO₂NR^{a}R^{c}, and a 4-9 membered mono- or bi-cyclic N-heterocycloalkyl ring that can be optionally substituted with 1-3 C₁-C₃ alkyl and can be optionally interrupted by O, S, NR^{c}, or C═O;

R^{e} is selected from the group consisting of hydrogen, C₁-C₆ alkyl, C₂-C₆ alkenyl, phenyl, and phenylalkyl, wherein said alkyl, alkenyl, or phenyl group can either be unsubstituted or substituted with 1-3 substituents independently selected from the group consisting of C₁-C₃ alkyl, OH, O(C₁-C₄ alkyl), NH₂, NH(C₁-C₄ alkyl), N(C₁-C₄ alkyl)₂, halo, CN, NO₂, CO₂H, CO₂(C₁-C₄ alkyl), C(O)H, and C(O)(C₁-C₄ alkyl);

Y is selected from the group consisting of CR^{b}R^{c}, C₂-C₆ alkylene and C₂-C₆ alkenylene, wherein said alkylene and alkenylene linkers can be optionally interrupted by O, S, or NR^{c}; and

Z is selected from the group consisting of O, S, NR^{c}, C═O, O(C═O), (C═O)O, NR^{c}(C═O) or (C═O)NR^{c};

or pharmaceutically acceptable salt or ester thereof.

In some embodiments, the agonist can comprise a pyranoflavonoid agonist. Examples of such agonists are described, for example, in International Publication No. WO 2002/058639, which is hereby incorporated by reference in its entirety.

In some embodiments, the agonist can comprise a dibenzochromene agonist. Examples of such agonists are described, for example, in U.S. Pat. No. 7,157,492 to Mewshaw et al. and International Publication No. WO 2003/051863 to Mewshaw et al., each of which is hereby incorporated by reference in its entirety.

In some embodiments, the agonist can comprise a 6H-chromeno[4,3-b]quinoline agonist. Examples of such agonists are described, for example, in U.S. Patent Application Publication No. 2006/0052410 to Vu et al., which is hereby incorporated by reference in its entirety.

In some embodiments, the agonist can comprise a tetracycle containing benzofuran. Examples of such agonists are described, for example, in U.S. Patent Application Publication No. 2006/0004087 to Miller et al., which is hereby incorporated by reference in its entirety. In one example, the agonist can comprise WAY-358.

In some embodiments, the agonist can comprise a cycloalkyl-substituted benzopyran. Examples of such agonists are described, for example, in International Publication No. WO 2004/094400 to Durst et al., which is hereby incorporated by reference in its entirety.

In some embodiments, the agonist can be defined by Formula XXXI below

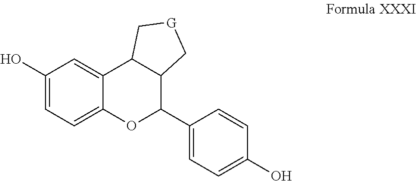

wherein

G is CHC₁-C₆ alkyl, C═O, CHOH, CF₂, C(OH)CF₃, CHCF₃, CH(OH)C₁-C₆ alkyl, CH—OC₁-C₆ alkyl, CH—O(CO)C₁-C₆ alkyl, CHF, CHCN, CHC₂-C₄ alkenyl, CHC₂-C₄ alkynyl, CHbenzyl, difluoromethylene, O, or S(O)ₙ, wherein n is 0-2;

including enantiomers thereof and pharmaceutically acceptable salts thereof.

In some embodiments, the agonist can comprise one or more of the following: (a) (2S, 3aS, 4R, 9bR)-4-(4-Hydroxy-phenyl)-2-methyl-1,2,3,3a,4,9b-hexahydro-cyclopenta[c]chromen-8-ol; (b) (2R, 3aR, 4S, 9bS)-4-(4-Hydroxy-phenyl)-2-methyl-1,2,3,3a,4,9b-hexahydro-cyclopenta[c]chromen-8-ol; (c) (2R, 3aR, 4S, 9bS)-2-tert-Butyl-4-(4-hydroxy-phenyl)-1,2,3,3a,4,9b-hexahydro-cyclopenta[c]chromen-8-ol; (d) (2S, 3aS, 4R, 9bR)-2-tert-Butyl-4-(4-hydroxy-phenyl)-1,2,3,3a,4,9b-hexahydro-cyclopenta[c]chromen-8-ol; (e) (3aS, 4S, 9bS)-4-(4-Hydroxy-phenyl)-1,3a,4,9b-tetrahydro-3H-2,5-dioxa-cyclopenta[a]naphthalen-8-ol; (f) (3aR, 4R, 9bR)-4-(4-Hydroxy-phenyl)-1,3a,4,9b-tetrahydro-3H-2,5-dioxa-cyclopenta[a]naphthalen-8-ol; (g) (3aR, 4S, 9bS)-4-(4-Hydroxy-phenyl)-1,3a,4,9b-tetrahydro-3H-5-oxa-2-thia-cyclopenta[a]naphthalen-8-ol; (h) (3aS, 4R, 9bR)-4-(4-Hydroxy-phenyl)-1,3a,4,9b-tetrahydro-3H-5-oxa-2-thia-cyclopenta[a]naphthalen-8-ol; (i) (2S, 3aR, 4S, 9bS)-4-(4-Hydroxy-phenyl)-2-oxo-1,2,3,3a,4,9b-hexahydro-5-oxa-2λ⁴-thia-cyclopenta[a]naphthalen-8-ol; (j) (2R, 3aS, 4R, 9bR)-4-(4-Hydroxy-phenyl)-2-oxo-1,2,3,3a,4,9b-hexahydro-5-oxa-2λ⁴-thia-cyclopenta[a]naphthalen-8-ol; (k) (3aR, 4S, 9bS)-4-(4-Hydroxy-phenyl)-2,2-dioxo-1,2,3,3a,4,9b-hexahydro-5-oxa-2λ⁶-thia-cyclopenta[a]naphthalen-8-ol; (l) (3aS, 4R, 9bR)-4-(4-Hydroxy-phenyl)-2,2-dioxo-1,2,3,3a,4,9b-hexahydro-5-oxa-2λ⁶-thia-cyclopenta[a]naphthalen-8-ol; (m) (3aR, 4S, 9bS)-8-Hydroxy-4-(4-hydroxy-phenyl)-1,3a,4,9b-tetrahydro-3H-cyclopenta[c]chromen-2-one; (n) (3aS, 4R, 9bR)-8-Hydroxy-4-(4-hydroxy-phenyl)-1,3a,4,9b-tetrahydro-3H-cyclopenta[c]chromen-2-one; (o) (2S, 3aR, 4S, 9bS)-4-(4-Hydroxy-phenyl)-1,2,3,3a,4,9b-hexahydro-cyclopenta[c]chromene-2,8-diol; (p) (2R, 3aS, 4R, 9bR)-4-(4-Hydroxy-phenyl)-1,2,3,3a,4,9b-hexahydro-cyclopenta[c]chromene-2,8-diol; (q) (3aR, 4S, 9bS)-2,2-Difluoro-4-(4-hydroxy-phenyl)-1,2,3,3a,4,9b-hexahydro-cyclopenta[c]chromen-8-ol; (r) (3aS, 4R, 9bR)-2,2-Difluoro-4-(4-hydroxy-phenyl)-1,2,3,3a,4,9b-hexahydro-cyclopenta[c]chromen-8-ol; (s) (2S, 3aR, 4S, 9bS)-4-(4-Hydroxy-phenyl)-2-trifluoromethyl-1,2,3,3a,4,9b-hexahydro-cyclopenta[c]chromen-8-ol; (t) (2R, 3aS, 4R, 9bR)-4-(4-Hydroxy-phenyl)-2-trifluoromethyl-1,2,3,3a,4,9b-hexahydro-cyclopenta[c]chromen-8-ol; (u) (2R, 3aS, 4S, 9bS)-4-(4-Hydroxy-phenyl)-2-trifluoromethyl-1,2,3,3a,4,9b-hexahydro-cyclopenta[c]chromen-8-ol; (v) (2S, 3aS, 4R, 9bR)-4-(4-Hydroxy-phenyl)-2-trifluoromethyl-1,2,3,3a,4,9b-hexahydro-cyclopenta[c]chromen-8-ol; (w) (2R, 3aR, 4S, 9bS)-2-Ethyl-4-(4-hydroxy-phenyl)-1,2,3,3a,4,9bR-hexahydro-cyclopenta[c]chromene-2,8-diol; (x) (2S, 3aS, 4R, 9bR)-2-Ethyl-4-(4-hydroxy-phenyl)-1,2,3,3a,4,9bR-hexahydro-cyclopenta[c]chromene-2,8-diol; (y) (2S, 3aS, 4R, 9bR)-2-Ethyl-4-(4-hydroxy-phenyl)-1,2,3,3a,4,9b-hexahydro-cyclopenta[c]chromen-8-ol; (z) (2S, 3aR, 4S, 9bS)-2-Ethyl-4-(4-hydroxy-phenyl)-1,2,3,3a,4,9b-hexahydro-cyclopenta[c]chromen-8-ol; (aa) (2S, 3aR, 4S, 9bS)-2-Ethyl-4-(4-hydroxy-phenyl)-1,2,3,3a,4,9b-hexahydro-cyclopenta[c]chromen-8-ol; (bb) (2R, 3aR, 4S, 9bS)-2-Ethyl-4-(4-hydroxy-phenyl)-1,2,3,3a,4,9b-hexahydro-cyclopenta[c]chromen-8-ol; (cc) (2S, 3aS, 4R, 9bR)-4-(4-Hydroxy-phenyl)-2-methoxy-1,2,3,3a,4,9b-hexahydro-cyclopenta[c]chromen-8-ol; (dd) (2R, 3aR, 4S, 9bS)-4-(4-Hydroxy-phenyl)-2-methoxy-1,2,3,3a,4,9b-hexahydro-cyclopenta[c]chromen-8-ol; (ee) (2S, 3aS, 4R, 9bR)-Acetic acid 8-hydroxy-4-(4-hydroxy-phenyl)-1,2,3,3a,4,9b-hexahydro-cyclopenta[c]chromen-2-yl ester; (ff) (2R, 3aR, 4S, 9bS)-Acetic acid 8-hydroxy-4-(4-hydroxy-phenyl)-1,2,3,3a,4,9b-hexahydro-cyclopenta[c]chromen-2-yl ester; (gg) (2R, 3aS, 4R, 9bR)-2-Fluoro-4-(4-hydroxy-phenyl)-1,2,3,3a,4,9b-hexahydro-cyclopenta[c]chromen-8-ol; (hh) (2S, 3aR, 4S, 9bS)-2-Fluoro-4-(4-hydroxy-phenyl)-1,2,3,3a,4,9b-hexahydro-cyclopenta[c]chromen-8-ol; (ii) (2S, 3aS, 4R, 9bR)-2-Fluoro-4-(4-hydroxy-phenyl)-1,2,3,3a,4,9b-hexahydro-cyclopenta[c]chromen-8-ol; (j) (2R, 3aR, 4S, 9bS)-2-Fluoro-4-(4-hydroxy-phenyl)-1,2,3,3a,4,9b-hexahydro-cyclopenta[c]chromen-8-ol; (kk) (2R, 3aR, 4S, 9bS)- and (2S, 3aS, 4R, 9bR)-8-Hydroxy-4-(4-hydroxy-phenyl)-1,2,3,3a,4,9b-hexahydro-cyclopenta[c]chromene-2-carbonitrile; (ll) (2S, 3aR, 4S, 9bS)- and (2R, 3aS, 4R, 9bR)-8-Hydroxy-4-(4-hydroxy-phenyl)-1,2,3,3a,4,9b-hexahydro-cyclopenta[c]chromene-2-carbonitrile; (mm) (3aR, 4S, 9bS)- and (3aS, 4R, 9bR)-4-(4-Hydroxy-phenyl)-2-methylene-1,2,3,3a,4,9b-hexahydro-cyclopenta[c]chromen-8-ol; (nn) (3aR, 4S, 9bS)- and (3aS, 4R, 9bR)-2-Difluoromethylene-4-(4-hydroxy-phenyl)-1,2,3,3a,4,9b-hexahydro-cyclopenta[c]chromen-8-ol; (oo) (3aR, 4S, 9bS)- and (3aS, 4R, 9bR)-2-Ethynyl-4-(4-hydroxy-phenyl)-1,2,3,3a,4,9b-hexahydro-cyclopenta[c]chromen-8-ol; (pp) 2-Butyl-4-(4-hydroxy-phenyl)-1,2,3,3a,4,9b-hexahydro-cyclopenta[c]chromen-8-ol; (qq) 4-(4-Hydroxy-phenyl)-2-propyl-1,2,3,3a,4,9b-hexahydro-cyclopenta[c]chromen-8-ol; (rr) 2-Ethyl-4-(4-hydroxy-phenyl)-1,2,3,3a,4,9b-hexahydro-cyclopenta[c]chromen-8-ol; (ss) 2-Benzyl-4-(4-hydroxy-phenyl)-1,2,3,3a,4,9b-hexahydro-cyclopenta[c]chromen-8-ol; enantiomers thereof; or pharmaceutically acceptable salts thereof.

In some embodiments, the agonist can comprise a steroidal derivative. Examples of such agonists are described, for example, in International Publication No. WO 2019/035061 to Micalizio et al., which is hereby incorporated by reference in its entirety.

In some embodiments, the agonist can be defined by Formula XXXIIA, XXXIIB, XXXIIC, or XXXIID below

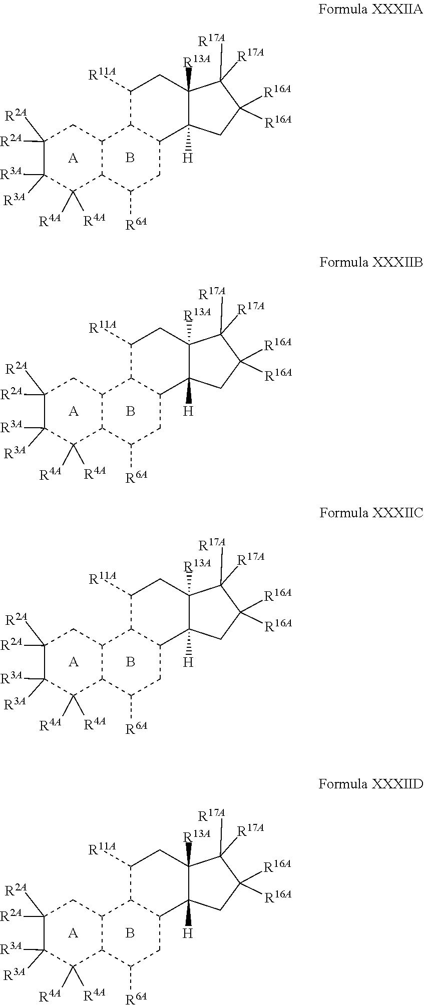

wherein

each R^{2A} and each R^{4A} is independently absent or, when present, selected from the group consisting of hydrogen, C₁-C₁₀ alkyl, C₂-C₁₀ alkenyl, C₂-C₁₀ alkynyl, C₁-C₁₀ haloalkyl, halogen, hydroxy, —OR^{AX}, —SR^{AY}, —S(O)₂NR^{Z1}R^{Z2}, —S(O)₂R^{Z1}, —S(O)R^{Z1}, —NR^{Z1}R^{Z2}, N(R^{Z1})C(O)R^{Z2}, —N(R^{Z1})S(O)₂R^{Z2}, C₆-C₁₀ aryl, and 5- to 10-membered heteroaryl, or two R^{2A} together or two R^{4A} together form an oxo,

wherein R^{AX} is C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, —C(O)—O—C₁-C₁₀ alkyl, —C(O)—O—C₆-C₁₀ aryl, —C(O)—O-heteroaryl, —C(O)—NR^{Z1}R^{Z2}, —S(O)₂R^{Z1}, C₆-C₁₀ aryl, or 5- to 10-membered heteroaryl, wherein RAY is hydrogen, C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl, wherein each of R^{Z1} and R^{Z2} are independently hydrogen, C₁-C₆ alkyl, C₂-C₁₀ alkenyl, C₂-C₁₀ alkynyl, C₁-C₁₀ haloalkyl, C₆-C₁₀ aryl, 5- to 10-membered heteroaryl, hydroxy, or C₁-C₆ alkoxy;

each R^{3A} is independently absent or, when present, selected from the group consisting of hydrogen, C₁-C₁₀ alkyl, C₂-C₁₀ alkenyl, C₂-C₁₀ alkynyl, C₁-C₁₀ haloalkyl, halogen, —OR^{AX}, —SR^{AY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆-C₁₀ aryl, and 5- to 10-membered heteroaryl;

R^{6A} is selected from the group consisting of hydrogen, C₁-C₁₀ alkyl, C₂-C₁₀ alkenyl, C₂-C₁₀ alkynyl, C₁-C₁₀ haloalkyl, halogen, oxygen, boronic acid, boronic acid ester, —OR^{BX}, —SR^{BY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆-C₁₀ aryl, 5- to 10-membered heteroaryl, C₆-C₁₀ aryl C₁-C₆ alkyl, C₆-C₁₀ aryl C₂-C₆ alkenyl, and C₆-C₁₀ aryl C₂-C₆ alkynyl,

wherein R^{BX} is C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl,

wherein R^{BY} is hydrogen, C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl;

R^{11A} is selected from the group consisting of hydrogen, oxygen, and OR^{CX},

wherein R^{CX} is C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl;

R^{13A} is selected from the group consisting of C₁-C₆ alkyl and C₆-C₁₀ aryl C₁-C₆ alkyl, wherein the C₆-C₁₀ aryl is optionally substituted one or more halogen, C₁-C₆ alkyl, C₁-C₆ haloalkyl, or C₁-C₆ alkoxy;

each R^{16A} is independently selected from the group consisting of hydrogen, hydroxy, —OR^{DX}, —SR^{DY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, and —C(O)—C₁-C₁₀ alkyl, or two R^{16A} together form an oxo,

wherein R^{DX} is C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl, wherein R^{DY} is hydrogen, C₁-C₆ alkyl, C₂-C₁₀ alkenyl, C₂-C₁₀ alkynyl, C₁-C₁₀ haloalkyl, —C(O)—C₁-C₁₀ alkyl, —C(O)—C₆-C₁₀ aryl, —C(O)-heteroaryl, C₆-C₁₀ aryl, or 5- to 10-membered heteroaryl;

each R^{17A} is independently selected from the group consisting of hydrogen, C₁-C₁₀ alkyl, C₂-C₁₀ alkenyl, C₂-C₁₀ alkynyl, C₁-C₁₀ haloalkyl, and halogen, or two R^{17A} together form an oxo;

each dotted line independently represents a single bond or a double bond;

the A ring is saturated, partially unsaturated, or completely unsaturated; and the B ring is saturated, partially unsaturated, or completely unsaturated;

wherein any C₆-C₁₀ aryl or 5- to 10-membered heteroaryl is optionally substituted with one or more halogen, C₁-C₆ alkyl, C₁-C₆ haloalkyl, or C₁-C₆ alkoxy.

In certain embodiments, the agonist can be the compound defined below.

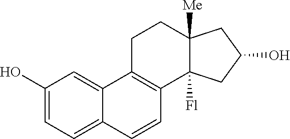

In some embodiments, the agonist can comprise a 4-cycloheptylphenol, such as the compound shown below.

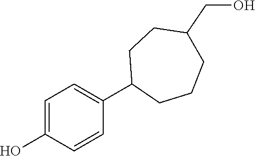 [SMILES: OCC1CCCC(c2ccc(O)cc2)CC1]

In some embodiments, the agonist can comprise one of the compounds shown below.

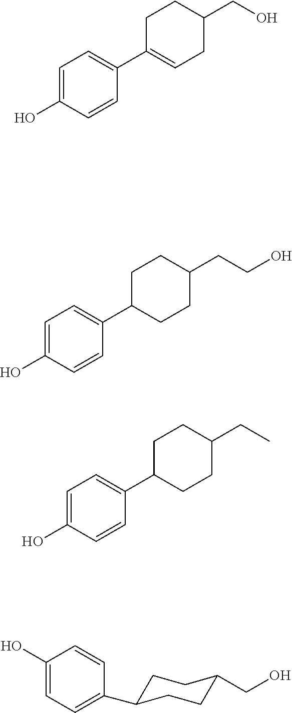 [SMILES: OCC1CC=C(c2ccc(O)cc2)CC1 ; OCCC1CCC(c2ccc(O)cc2)CC1 ; CCC1CCC(c2ccc(O)cc2)CC1 ; OCC1CCC(c2ccc(O)cc2)CC1]

In some embodiments, the agonist can comprise a polyhydroxyphthalazinone. Examples of such agonists are described, for example, in International Publication No. WO 2018/214736 to Liang et al., which is hereby incorporated by reference in its entirety.

In some embodiments, the agonist can be a compound defined by Formula XXXIII below

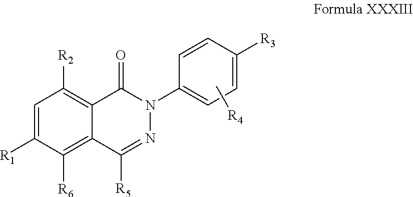

wherein R¹, R², R³, and R⁴ are each independently hydrogen, hydroxy, C₁-C₃ alkoxy or halogen;

R⁵ is hydrogen, C₁-C₄ alkyl, C₁-C₄ halogenated alkyl, phenyl or cyano; and

R⁶ is hydrogen or halogen.

In some embodiments, the halogen can be fluorine, chlorine or bromine.

In certain embodiments, R¹, R², and R³ are hydroxy, R⁴ and R⁶ are hydrogen; and R⁵ is hydrogen, C₁-C₄ alkyl, C₁-C₄ halogenated alkyl, phenyl or cyano.

In certain embodiments, R¹, R², and R³ are hydroxy, R⁴ and R⁶ are hydrogen; and R⁵ is chlorine or bromine.

In certain embodiments, R¹, R², and R³ are hydroxy; R⁴ is hydrogen or halogen; R⁵ is hydrogen; R⁶ is chlorine or bromine.

In some embodiments, the agonist can comprise the compound below.

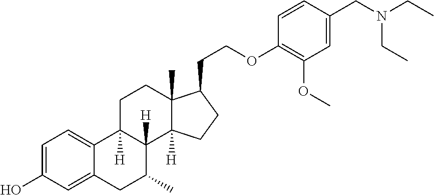

Such agonists are described, for example, in International Publication No. WO 2017/010515 to Nakao et al., which is hereby incorporated by reference in its entirety.

In some embodiments, the agonist can comprise an isoflavone such as genistein, the structure of which is shown below.

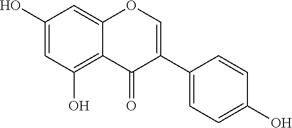 [SMILES: O=c1c(-c2ccc(O)cc2)coc2cc(O)cc(O)c12]

In some examples, the agonist can comprise one of the compounds shown below.

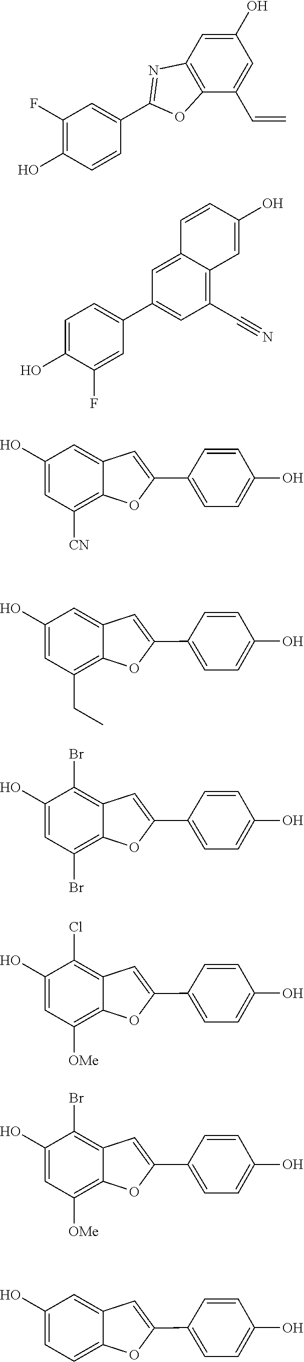

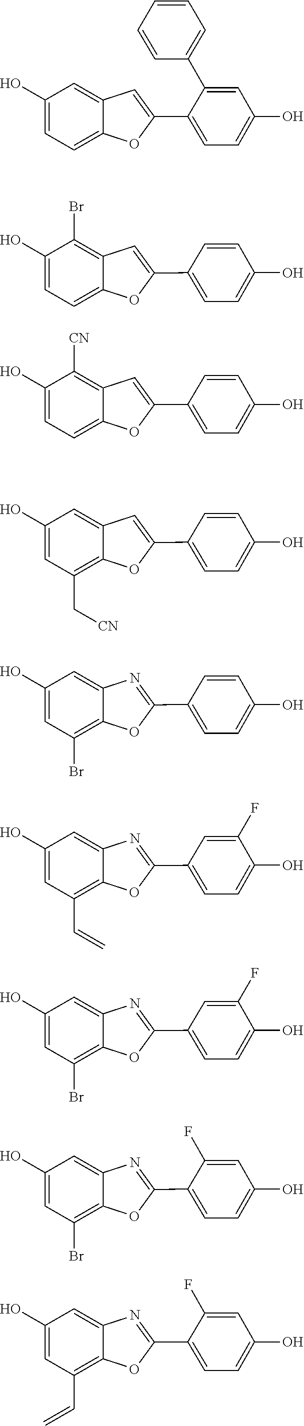

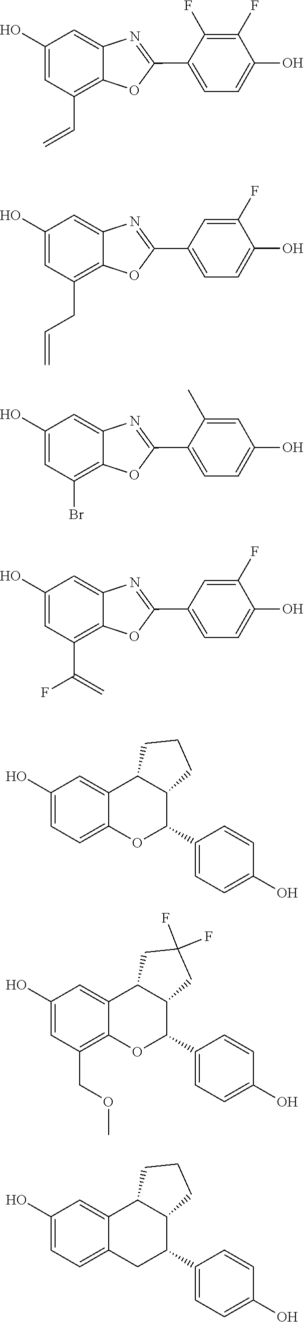

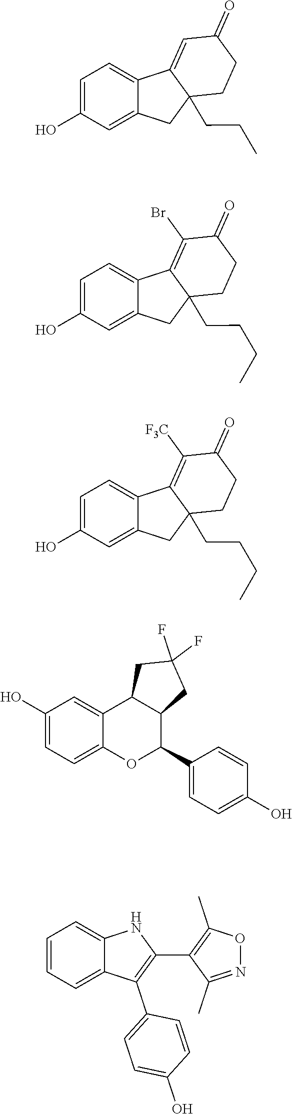

Methods of Use

ERβ agonists, including those described herein, can inhibit the activation and proliferation of CD4+ T-cells. As such, ERβ agonists can be administered to a subject to reduce circulating CD4+ T-cell levels (e.g., relative to levels prior to administration of the ERβ agonist, or relative to levels in a control not treated with the ERβ agonist). In some embodiments, the ERβ agonist can selectively reduce levels of circulating CD4+ T-cells while leaving the levels of other leukocytes substantially unchanged. For example, in some embodiments, the ERβ agonist can be administered in an effective amount to reduce circulating CD4+ T-cell levels in the subject (e.g., by at least 20%, 25%, 30%, 40%, 50%, 60%, 70%, 75%, or more) without significantly affecting circulating levels of neutrophils, monocytes, or B-cells (e.g., a change of less than 15%, less than 10%, or less than 5%). These reductions can be measured relative to levels prior to administration of the ERβ agonist, or relative to levels in a control not treated with the ERβ agonist.

CD4+ T-cells mediate wound-healing post-myocardial infarction (MI) but exacerbate left-ventricular (LV) remodeling during chronic heart failure (HF). Mechanisms that lead to this transition are unknown. However, without wishing to be bound by theory, it is believed that T-cells activate specific pathological signals to promote LV-remodeling during chronic HF. To identify such signals, limited cell RNA-sequencing of CD4+ T-cells sorted from the failing hearts (8 weeks post-MI) of male mice was performed and, surprisingly, activation of estrogen receptor (ER)-α signaling was observed. As ERα effects are antagonized by ERβ, ERβ agonists (including the ERβ agonists described herein) can be administered to modulate T-cell activity and LV remodeling.

As detailed in the Examples below, in vitro assays showed that an example ERβ agonist (Compound 1) dose dependently inhibited the activation and proliferation of T-cells sorted from male mice (IC₅₀ 3.4 μM). In vivo assays (60 mg/kg/day; oral) showed no overt toxicity and a significant reduction in circulating T cells without affecting neutrophils, monocytes, or B-cells indicating specificity against T-cells. Furthermore, this effect was specific to TCR-mediated T-cell activation and the drug did not affect T-cells stimulated with PMA/Ionomycin suggesting preferential inhibition of antigenically-activated T-cells.

To test therapeutic efficacy, male 10-12 week old mice underwent coronary ligation or sham operation and at 4 weeks post-MI randomized according to their cardiac function to receive either the vehicle or Compound 1 (60 mg/kg/day, oral) for the next 4 weeks. Consistently, at 8 weeks a significant reduction in T-cells in the circulation and the spleens of drug-treated mice was observed when compared with the vehicle treatment. Further, vehicle treated HF mice showed progressive LV dilatation with significantly increased end-diastolic and end-systolic volumes (EDV and ESV, respectively) from 4-8 weeks post-MI. Importantly, treatment with Compound 1 significantly inhibited these changes and blunted LV remodeling from 4-8 weeks post-MI. Significant reduction in tibia-normalized heart weights supported these results. These examples suggest that ERβ agonists can selectively inhibit T-cell activation and blunt pathological LV-remodeling during chronic HF.

Accordingly, provided herein are methods for treating or preventing chronic heart failure in a subject following myocardial infarction. These methods can comprise administering to the subject an ERβ agonist during a maladaptive remodeling phase following the myocardial infarction. Importantly, in some embodiments, the ERβ agonist is not administered to the subject during a healing phase or a repair phase preceding the maladaptive remodeling phase (i.e., administration of the ERβ agonist does not commence until the acute phase following MI is complete and the chronic phase has commenced).

In some embodiments, administration of the ERβ agonist commences at least 10 days following the myocardial infarction, such as at least 14 days following the myocardial infarction, at least 21 days following the myocardial infarction, at least 28 days following the myocardial infarction, at least 35 days following myocardial infarction, at least 42 days following myocardial infarction, at least 49 days following myocardial infarction, or at least 56 days following myocardial infarction.

These methods can further comprise assessing the subject to determine whether the subject has entered the maladaptive remodeling phase. This can be done via any suitable method. For example, in some embodiments, assessing the subject to determine whether the subject has entered the maladaptive remodeling phase can comprise measuring circulating CD4+ T-cell levels in the subject to determine when the subject has entered the maladaptive remodeling phase. In some embodiments, assessing the subject to determine whether the subject has entered the maladaptive remodeling phase can comprise detecting one or more biomarkers in the subject to determine when the subject has entered the maladaptive remodeling phase. Such biomarkers are known in the art, and include, for example, the relative levels of myosin heavy chain isoforms, GLUT-1 expression levels, alpha-actin expression levels, natriuretic peptide expression levels, galectin expression levels, caveolin expression levels, neuronal nitric oxide synthase expression levels, angiotensin-converting enzyme expression levels, GLUT-4 expression levels, SERCA2a expression levels, and a shift from glucose to fatty acid oxidation. In some embodiments, assessing the assessing the subject to determine whether the subject has entered the maladaptive remodeling phase can comprise echocardiography, ventriculography, nuclear magnetic resonance, or any combination thereof. Imaging techniques such as echocardiography and/or MRI can also be used to measure left-ventricular dilatation (increased end-diastolic and end-systolic volumes) as an indicator of LV remodeling.

In some embodiments, the ERβ agonist can be administered in an effective amount to inhibit activation and proliferation of CD4+ T-cells in the subject. As such, the ERβ agonist described herein can be administered to a subject to reduce circulating CD4+ T-cell levels (e.g., relative to levels prior to administration of the ERβ agonist, or relative to levels in a control not treated with the ERβ agonist). In some embodiments, the ERβ agonist can selectively reduce levels of circulating CD4+ T-cells while leaving the levels of other leukocytes substantially unchanged. For example, in some embodiments, the ERβ agonist can be administered in an effective amount to reduce circulating CD4+ T-cell levels in the subject (e.g., by at least 20%, 25%, 30%, 40%, 50%, 60%, 70%, 75%, or more) without significantly affecting circulating levels of neutrophils, monocytes, or B-cells (e.g., a change of less than 15%, less than 10%, or less than 5%). These reductions can be measured relative to levels prior to administration of the ERβ agonist, or relative to levels in a control not treated with the ERβ agonist.

In some embodiments, the ERβ agonist can be administered in an effective amount to alter morphological changes associated with CHF following MI. For example, in some embodiments, the ERβ agonist can be administered in an effective amount to decrease left ventricular (LV) remodeling in the subject. In some embodiments, the ERβ agonist can be administered in an effective amount to reduce changes is cardiac function associated with CHF following MI. For example, in some embodiments, the ERβ agonist can be administered in an effective amount to inhibit an increase in left ventricular end-diastolic volume in the subject, inhibit an increase in left ventricular end-systolic volume in the subject, or any combination thereof.

Also provided are methods of treating and preventing autoimmune disorders in which an ERβ agonist is administered to modulate T-cell activity (e.g., selectively modulate T-cell activity) in a subject. For example, provided herein are methods of treating or preventing graft-versus-host disease, multiple sclerosis (MS), and/or experimental autoimmune encephalomyelitis (EAE) in a subject that comprise administering to the subject an ERβ agonist.

When practicing these methods, the ERβ agonist can administered in an effective amount to inhibit activation and proliferation of CD4+ T-cells in the subject. For example, the ERβ agonist can be administered in an effective amount to reduce reducing circulating CD4+ T-cell levels in the subject. In certain embodiments, the ERβ agonist can be administered in an effective amount to reduce circulating CD4+ T-cell levels in the subject without significantly affecting circulating levels of neutrophils, monocytes, or B-cells.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection.

Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that a therapeutically effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use.

Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable excipient. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are set forth to illustrate the methods and results according to the disclosed subject matter. These examples are not intended to be inclusive of all aspects of the subject matter disclosed herein, but rather to illustrate representative methods and results. These examples are not intended to exclude equivalents and variations which are apparent to one skilled in the art.

### Example 1. Evaluation of Example ERβ Agonist for the Treatment of Heart Failure

Immune and inflammatory responses contribute to left ventricular (LV) remodeling after myocardial infarction (MI). Heightened levels of inflammatory cytokines promote cardiac remodeling and disease progression in heart failure (HF); however, clinical trials of TNF neutralization failed to show benefit and even revealed harm at high doses. These paradoxical results suggest a more complex role for inflammatory activation in heart failure than gleaned from cytokine levels alone. Activation of innate and adaptive immune cells underlies inflammatory responses in many chronic diseases. Monocytes, macrophages, and dendritic cells (DCs) mediate innate immune responses, whereas CD3+CD4+ helper and CD3+CD8+ cytotoxic T-cells arbitrate adaptive immunity. While innate immune cells comprise the first line of defense against acute injury, chronic inflammation often implies activation and clonal expansion of specialized effector T-cells following antigen presentation.

Previously published studies demonstrated that chronic ischemic heart failure can be characterized by global expansion of CD4+ T-cells in blood, spleen and the heart. Moreover, depletion of CD4+ T-cells (using anti-CD4 antibody) in heart failure mice prevented the deterioration of cardiac function and progression of left ventricular remodeling. These studies suggested that T-lymphocyte activation during chronic heart failure can play an important role in mediating pathological left ventricular (LV) remodeling associated with progressive cardiac dysfunction (increased end-diastolic and end-systolic volumes and ejection fraction), and heightened myocyte hypertrophy and fibrosis.

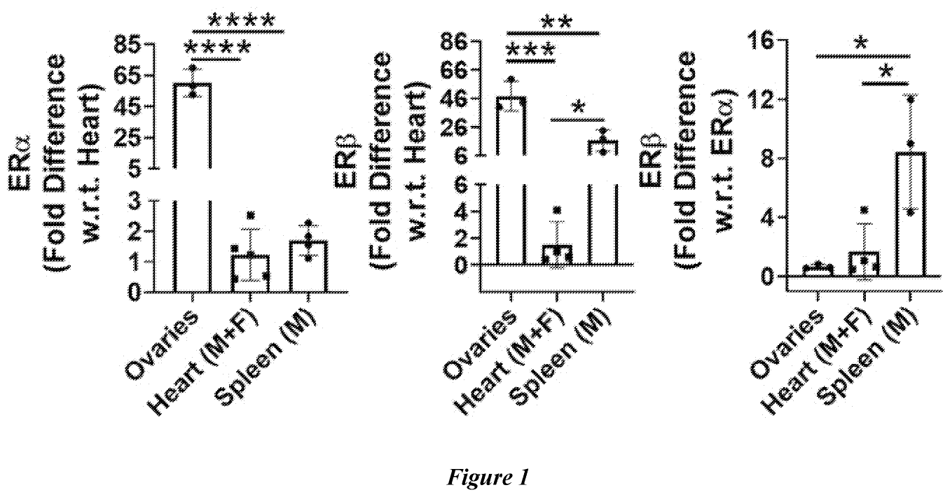

The gene expression for estrogen receptors (ERβ) α and β (ERα and ERβ) in the ovaries (positive control), hearts from both males and females (M+F), and male spleens is shown in FIG. 1. β-Actin was used as an internal control and fold changes with respect to the heart (left and middle panel) or ERα (right panel) are shown in FIG. 1. FIG. 1 shows that ERα expression is similar in the hearts and spleens and is almost negligible as compared to the ovaries. In contrast, ERβ gene expression in spleens is almost ⅓^{rd} of that in ovaries and is much higher as compared to the hearts (middle panel, FIG. 1). Moreover, the ratio of ERβ to ERα is much higher in spleens as compared to ovaries or the hearts (right panel, FIG. 1) suggesting preferential expression of ERβ as compared to ERα in splenocytes.

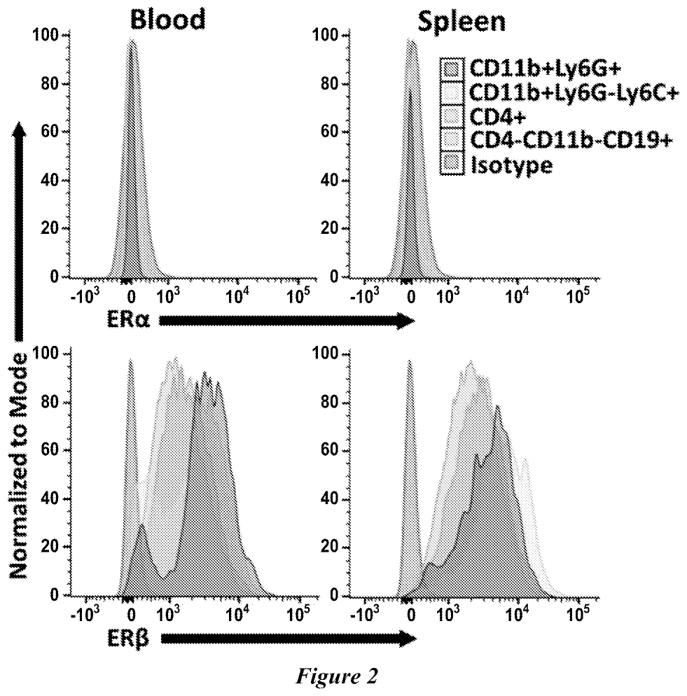

Representative flow cytometric histograms for ERα (top panels) and ERβ (bottom panels) expression in different circulating (left panels) and splenic (right panels) immune cells in a male mouse are shown in FIG. 2. Circulating and splenic immune cells do not express ERα, whereas several immune cell subsets were found to express significant levels of ERβ protein (FIG. 2). ERβ expression is highest in CD11b+ myeloid cells (Ly6G+ neutrophils and Ly6C+ monocytes/macrophages) followed by CD4+ helper T-cells, and is lowest in CD19+ B cells (FIG. 2).

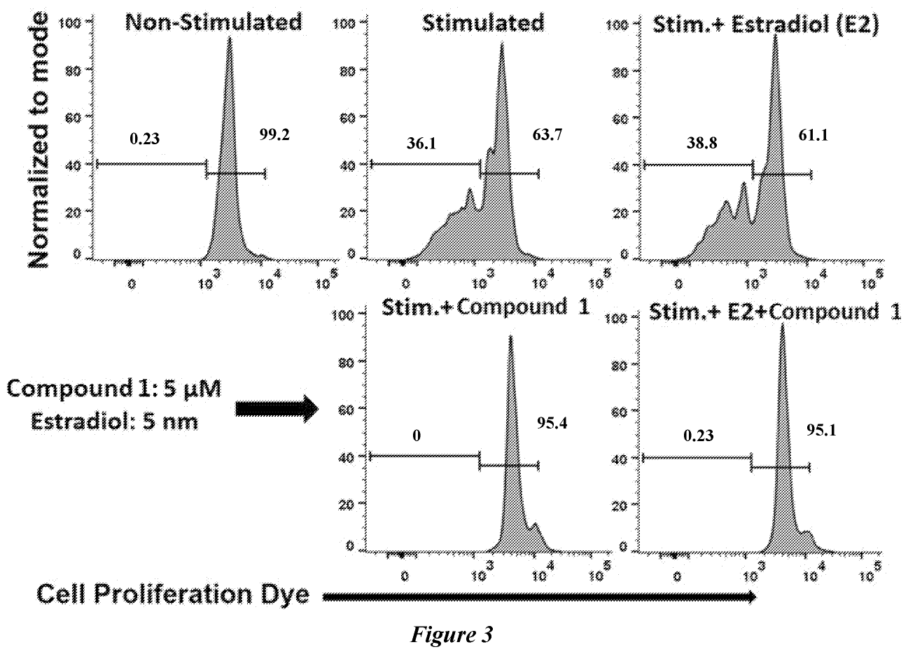

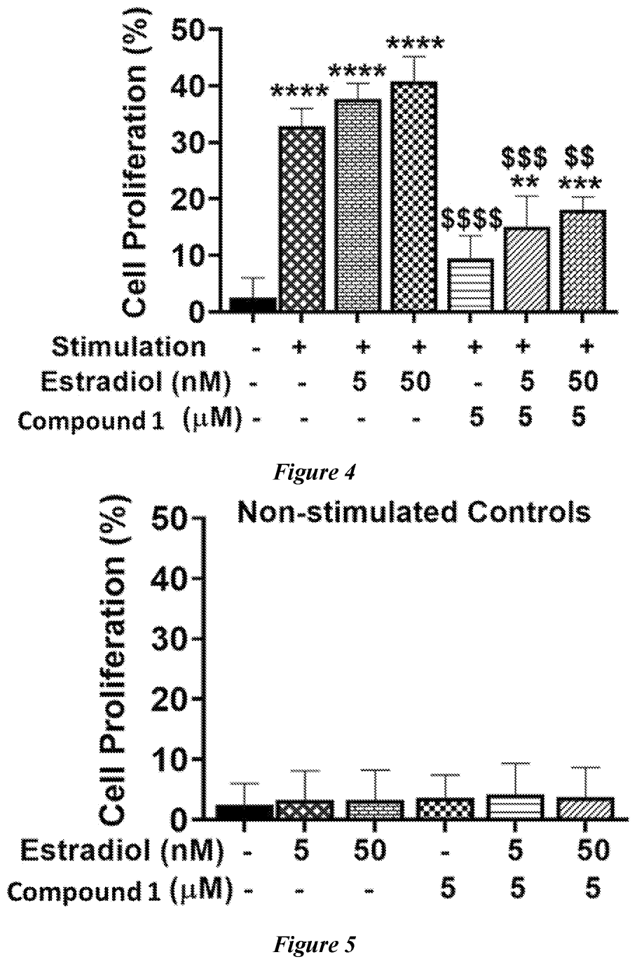

Representative flow cytometric histograms for cell trace violet labeled (CTV; a cell proliferation dye) CD4+ T-cells either unstimulated or CD3/CD28 TCR stimulated and treated with either Estradiol (5 and 50 nM) or Compound 1 (5 PM, an example ERβ agonist whose structure is shown below) or both are shown in FIG. 3. Peak patterns from high to low fluorescence intensity in stimulated cells represent halving of dye concentration in the cell membranes of the daughter cells with every successive cell division. Group quantitation for cell proliferation (%) measured as dye dilution with every successive cell division for stimulated and non-stimulated groups are shown in FIG. 4 and FIG. 5, respectively.

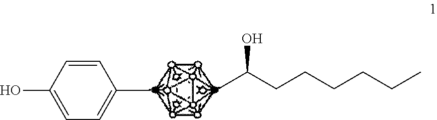

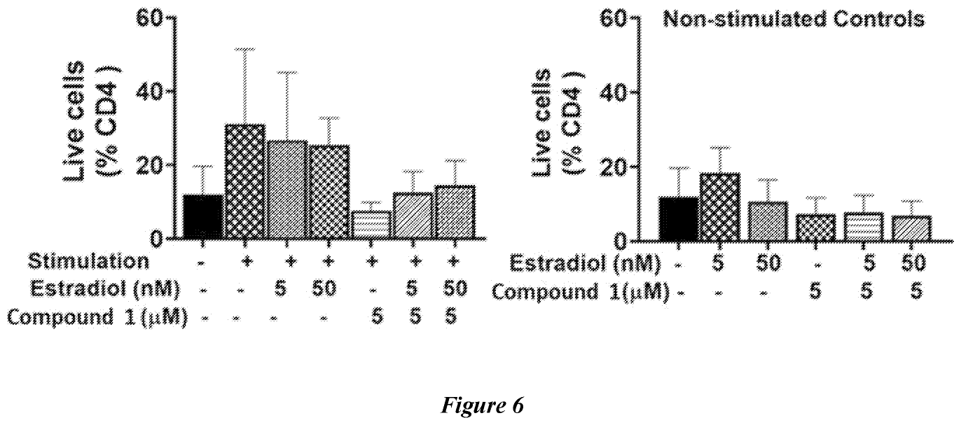

Cell survival of CD3/CD28 mediated in-vitro TCR stimulation with and without Compound 1 treatment are shown in FIG. 6. The results indicate that treatment with Compound 1 does not affect cell survival of T-cells stimulated with CD3/CD28 antibodies (FIG. 6).

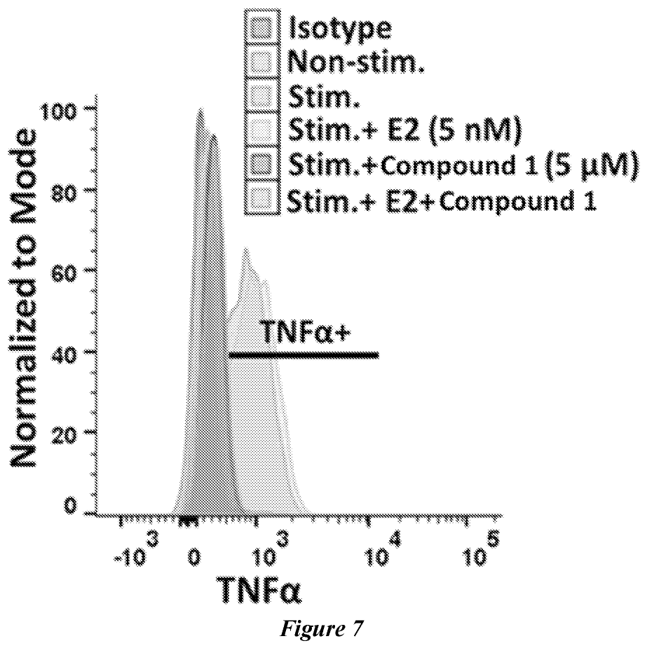

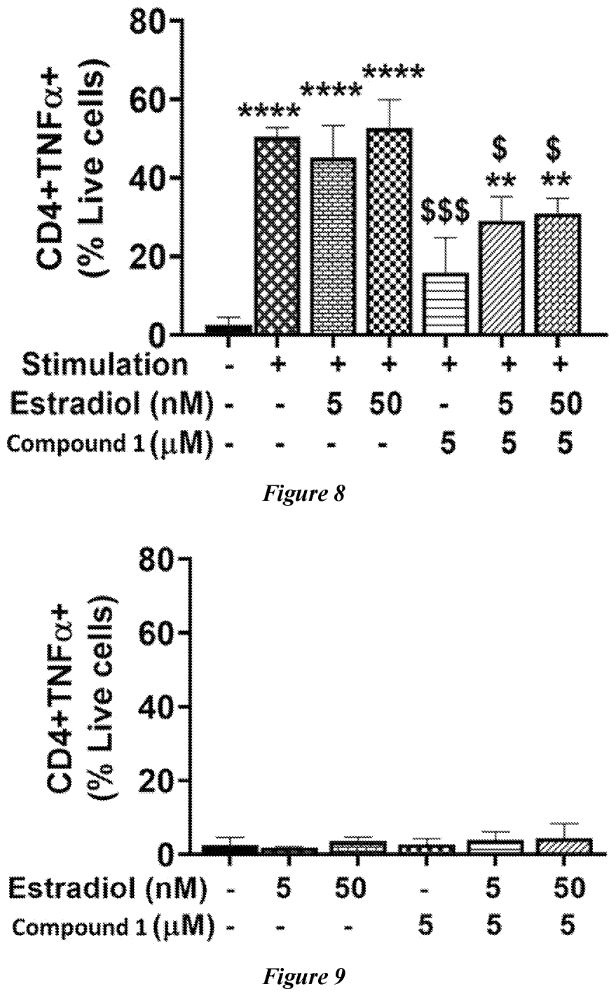

Representative flow cytometric histograms for TNFα expression in CD4+ T-cells either unstimulated or CD3/CD28 TCR stimulated and treated with either Estradiol (5 and 50 nM) or Compound 1 (5 μM) or both are shown in FIG. 7. Group quantitations for stimulated groups are shown in FIG. 8 and unstimulated control groups are shown in FIG. 9. The results indicate that treatment with Compound 1 suppressed the number of TNFα+ helper T-cells (CD4+).

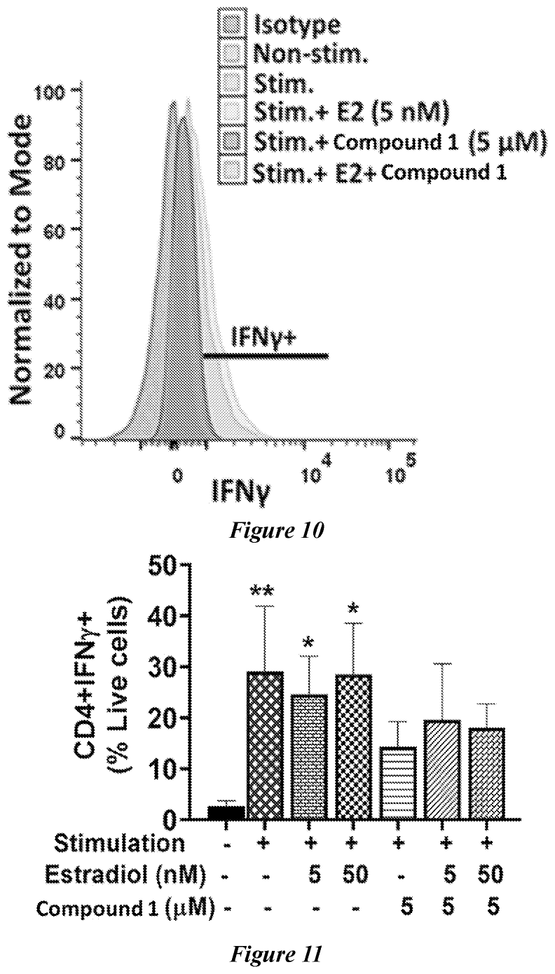

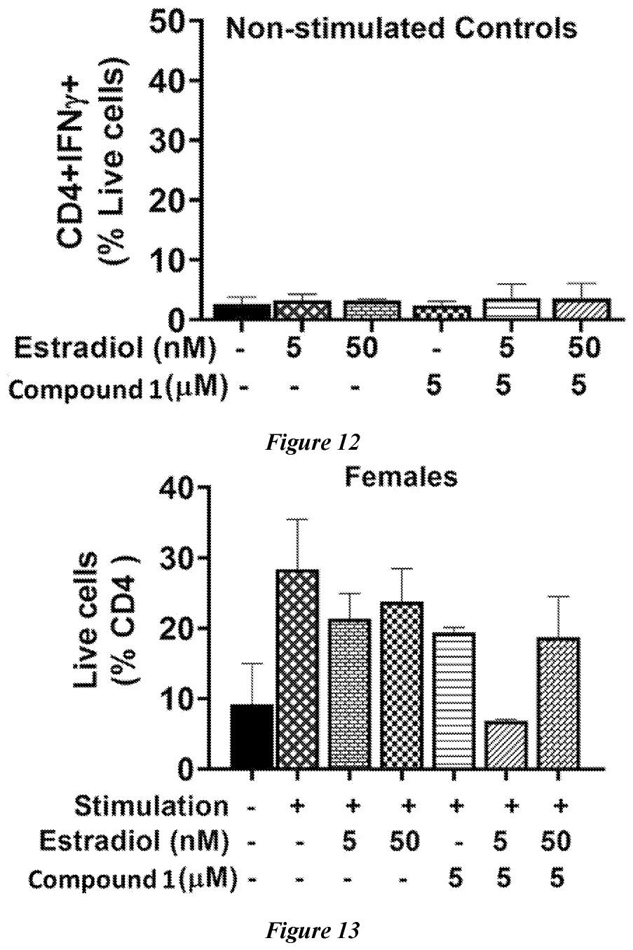

Representative flow cytometric histograms for IFNγ expression in CD4+ T-cells either unstimulated or CD3/CD28 TCR stimulated and treated with either Estradiol (5 and 50 nM) or Compound 1 (5 μM) or both are shown in FIG. 10. Group quantitations for stimulated groups are shown in FIG. 11 and unstimulated control groups are shown in FIG. 12. The results indicate that treatment with Compound 1 did not significantly affect the number of IFNγ+ helper T-cells (CD4+).

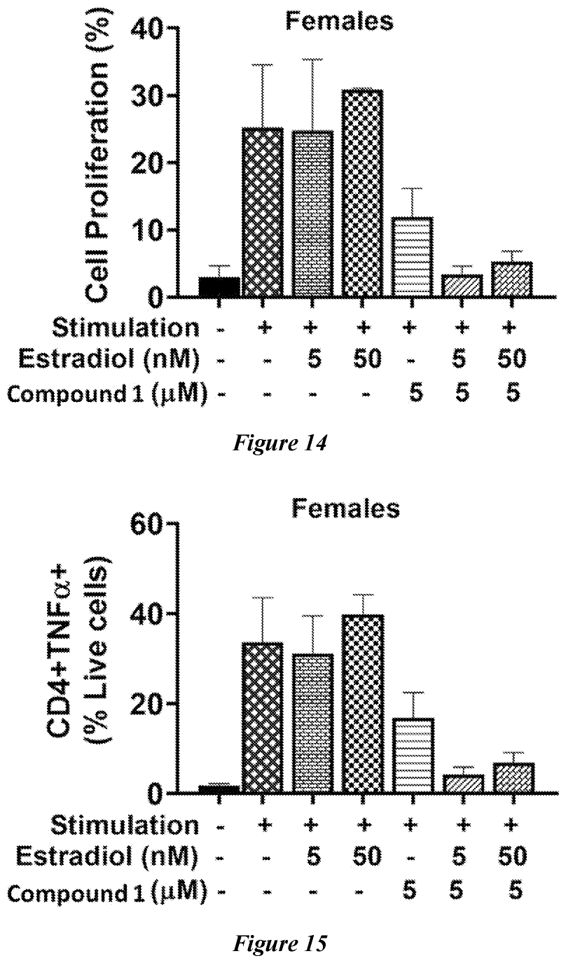

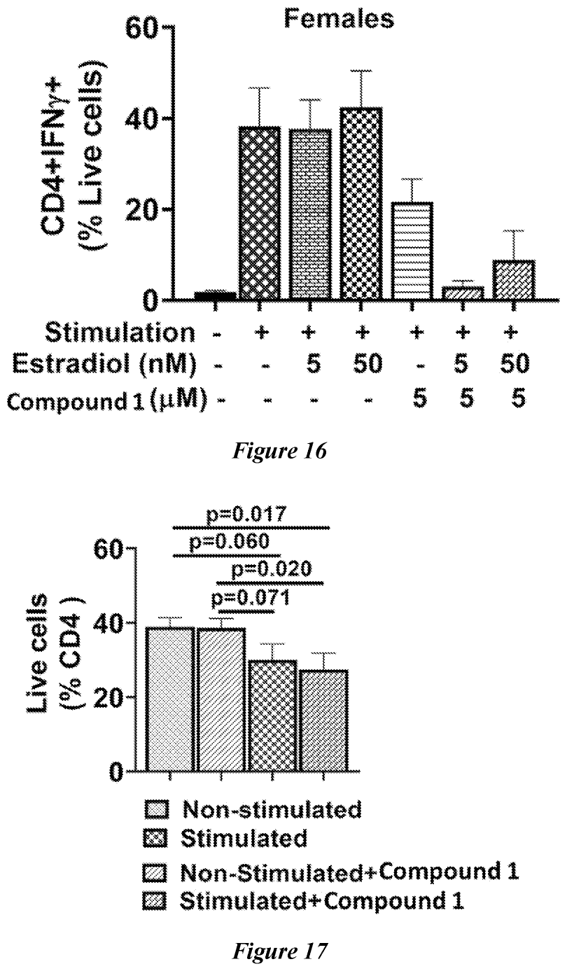

The results of CD3/CD28 TCR mediated in-vitro T-cell proliferation assays using T-cells from Female mice with and without Compound 1 are shown in FIG. 13-FIG. 16. The results show group quantitation for cell-survival (FIG. 13), proliferation (FIG. 14), TNFα+ helper T-cells (CD4+) (FIG. 15), and IFNγ+ helper T-cells (CD4+) (FIG. 16) either unstimulated or CD3/CD28 TCR stimulated and treated with either Estradiol (5 and 50 nM) or Compound 1 (5 μM) or both. The results suggest that treatment with Compound 1 suppresses cell proliferation, the number of TNFα+ helper T-cells (CD4+), and the number of IFNγ+ helper T-cells, without negatively affecting overall cell-survival.

The results of non-specific PMA/ionomycin mediated in-vitro T-cell activation with and without Compound 1 are shown in FIG. 17-FIG. 20.

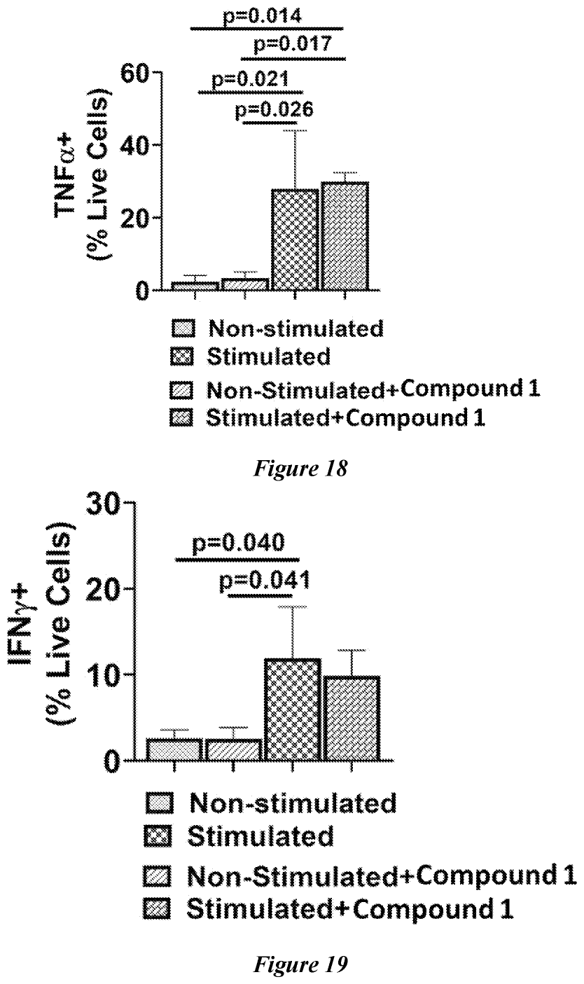

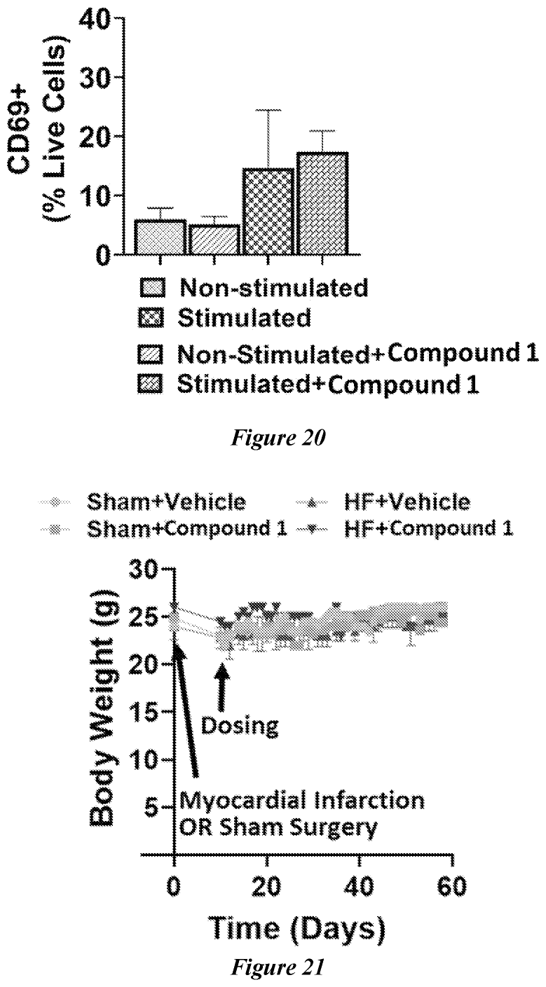

The results show group quantitation for cell-survival (FIG. 17), TNFα+ helper T-cells (CD4+) (FIG. 18), IFNγ+ helper T-cells (CD4+) (FIG. 19), and CD69+ helper T-cells (CD4+) (FIG. 20) either unstimulated or stimulated with PMA/Ionomycin and treated with Compound 1 (5 μM). The results indicate that Compound 1 treatment did not affect the levels of TNFα+, IFNγ+, or CD69+ T-cells post-PMA/Ionomycin stimulation, suggesting Compound 1 specifically affects CD3/CD28 TCR mediated T-cell activation pathways.

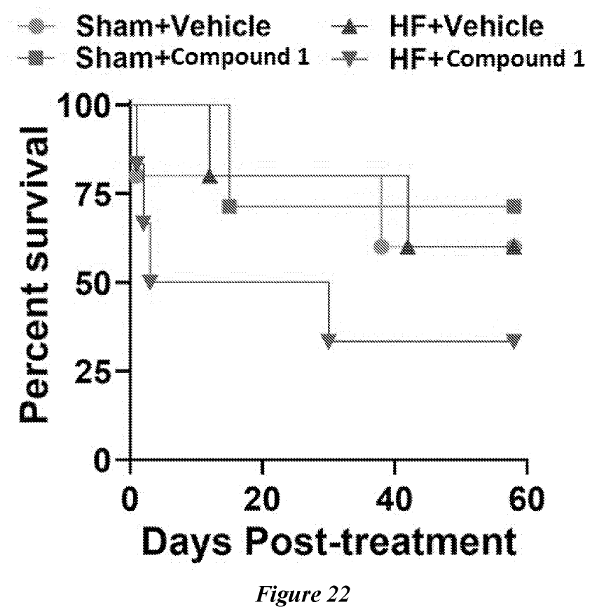

Next, in vivo treatment was tested using mice. First, treatment was started at 7 days post-infarction to inhibit immune activation in the acute phase (FIG. 27). The body weight of sham-operated and myocardial infarction (MI) mice treated with either vehicle control or Compound 1 is shown in FIG. 21 (day 0 corresponds to the day treatment started, e.g., 7 days post myocardial infarction). A Kaplan-Meier curve shows mortality rate in myocardial infarction and sham-operated mice treated with vehicle or drug in FIG. 22 (day 0 corresponds to the day treatment started, e.g., 7 days post myocardial infarction). As can be seen in FIG. 22, treatment with Compound 1 during the acute phase of immune activation increased mortality (decreased survival), indicating the immune response during the acute phase is important for adequate healing.

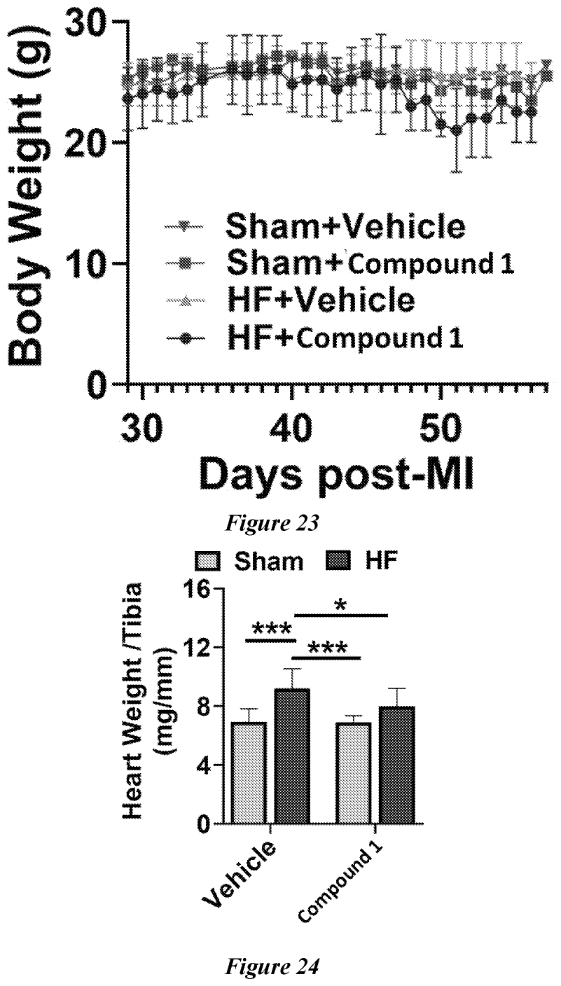

Accordingly, subsequent tests examined the effect of treatment at 28 days post-infarction to inhibit immune activation in the chronic (e.g., maladaptive remodeling) phase (FIG. 27). The body weight and tibia normalized heart weights of sham-operated and myocardial infarction (MI) mice treated with either vehicle control or Compound 1 28 days post-myocardial infarction are shown in FIG. 23 and FIG. 24, respectively. The results indicate that treatment with Compound 1 28 days post-myocardial infarction stopped the increase in heart weight (FIG. 24). Further, there was no significant increase in mortality associated with Compound 1 treatment 28 days post-myocardial infarction.

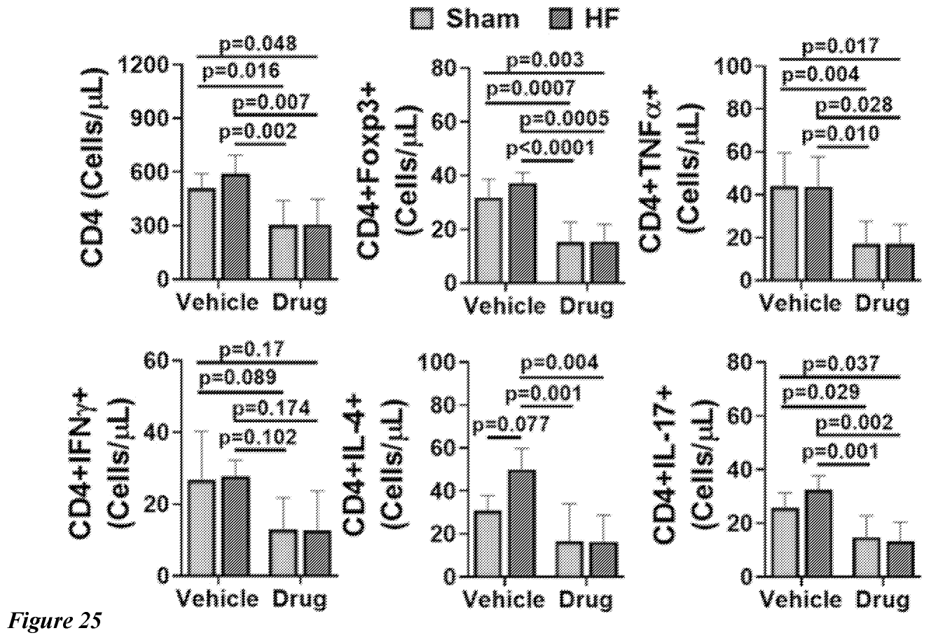

The levels of circulating CD4+ Helper T-cells (per μL blood) and its subsets viz CD4+Foxp3+(Tregs), CD4+TNFα+ cells, CD4+IFNγ+(Th1), CD4+IL-4+(Th2) and CD4+IL-17+(Th17) T-cells at 8 weeks post-surgery in mice treated with either vehicle or Compound 1 from 4 to at 8 weeks post-surgery are shown in FIG. 25. The results indicate that treatment with Compound 1 lead to a decrease in various types of CD4+ Helper T-cells.

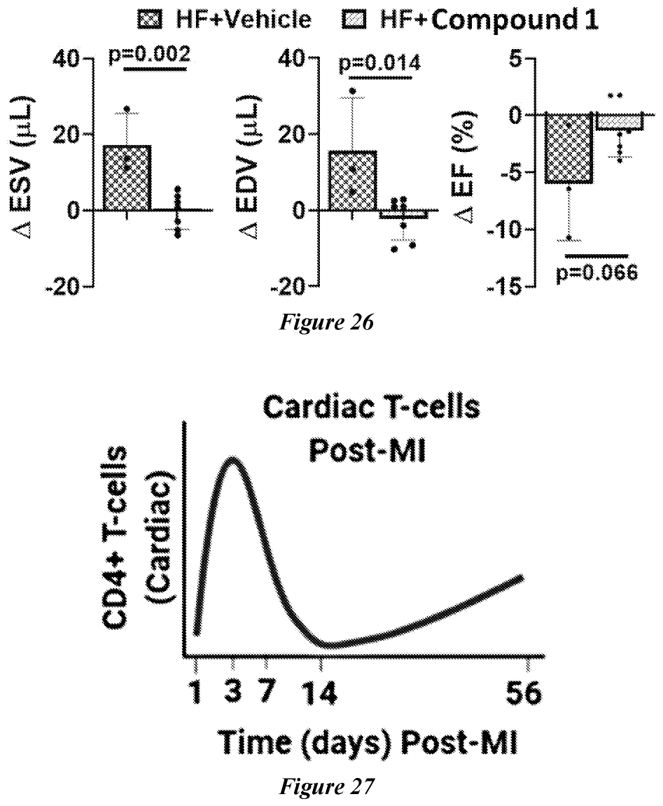

Quantitative group data for changes in left ventricular end-diastolic and end-systolic volumes (EDV and ESV) and ejection fraction (EF) in ligated mice before (4 weeks post-myocardial infarction) and after (8 weeks post-myocardial infarction) treatment with either vehicle or Compound 1 are shown in FIG. 26. The results indicate that the change in the end-systolic volume, end-diastolic volume, and ejection fraction were significantly less for ligated mice treated with Compound 1 than for the control.

Herein, a drug molecule Compound 1 was tested for its ability to inhibit T-lyphocyte activation and proliferation during chronic heart failure. It was found that Compound 1 inhibited CD3/CD28 TCR mediated T-cell activation under in-vitro T-cell proliferation assays. Administration of Compound 1 to heart failure mice (from 4 weeks postmyocardial infarction) reduced CD4+T-lymphocyte levels in the circulation and blunted progressive left ventricular remodeling measured at 8 weeks post-ligation.

### Example 2

Sustained and inappropriate inflammatory activation, as manifested by elevated levels of inflammatory cytokines such as tumor necrosis factor-α (TNF), contributes to disease progression in chronic heart failure (HF) (Ismahil et al. Circ Res. 2014; 114(2); 266-82). However, clinical trials of antibody-based anti-TNF therapies in heart failure failed to show clinical benefit (Anker et al. Int J Cardiol. 2002; 86:123-30). This suggests that inflammatory mechanisms in heart failure, and, by analogy, the approaches to therapeutic immunomodulation, are more complex and nuanced than gradations of cytokine responses in failing myocardium. As plasma cytokine levels are often a combined result of the interplay between activated immune cells, they may be less sensitive indicators of the underlying tissue events that are specifically mediated by inflammatory and immune cells. Indeed, levels of circulating and cardiac inflammatory cells (monocytes (Nahrendorf et al. J Exp Med. 2007; 204:3037-47), macrophages (Ismahil et al. Circ Res. 2014; 114(2); 266-82), and T-lymphocytes (Bansal et al. Circ Heart Fail. 2017; 10:e003688)) are increased during both the acute and chronic phases of heart failure, necessitating spatio-temporal dissection and identification of specific molecular signatures that can be targeted to restrain activation of pathological immune cells to achieve therapeutic immune-modulation.

Recent studies have established complex interrelationships between innate (e.g., monocytes/macrophages and dendritic cells) and adaptive immune cells (e.g., T-lymphocytes) in the regulation of tissue remodeling (Ismahil et al. Circ Res. 2014; 114(2); 266-82). Both the acute and chronic phases of left ventricular (LV) remodeling are influenced by cardiac infiltration of dendritic cells (Ismahil et al. Circ Res. 2014; 114(2); 266-82), monocytes (Nahrendorf et al. J Exp Med. 2007; 204:3037-47), macrophages (Ismahil et al. Circ Res. 2014; 114(2); 266-82), and CD4⁺ helper T-lymphocytes (Bansal et al. Circ Heart Fail. 2017; 10:e003688). Importantly, in contrast to early (7 days) cardiac remodeling after infarction, increased levels of pro-inflammatory iNOS⁺ and TNF⁺ innate (Kingery et al. Basic Res Cardiol. 2017; 112:19) and adaptive immune (Bansal et al. Circ Heart Fail. 2017; 10:e003688) cells in chronic heart failure is indicative of complex pathological modulation of the global immune cell networks. Furthermore, adoptive transfer of splenocytes (Ismahil et al. Circ Res. 2014; 114(2); 266-82) or splenic CD4⁺ T-cells (Bansal et al. Circ Heart Fail. 2017; 10:e003688) from heart failure mice induced significant left ventricular remodeling and cardiac dysfunction in naïve mice, whereas splenectomy (Ismahil et al. Circ Res. 2014; 114(2); 266-82) or antibody-mediated depletion of CD4⁺ T-cells (Bansal et al. Circ Heart Fail. 2017; 10:e003688) inhibited left ventricular remodeling and improved cardiac function in heart failure mice. These studies indicate that chronic ischemic heart failure is a state of global immune cell activation and expansion in the heart, blood, spleen, and lymph nodes (LNs). Several studies also show that estrogen receptors (ER) α and β are expressed on myeloid (monocytes/macrophages/dendritic cells) and lymphoid (B cells and T-cells) immune cells (Kovats S. Cell Immunol. 2015; 294:63-9). Furthermore, ERβ activation inhibits TNFα mediated NF-kB translocation (Xing et al. PLoS One. 2012; 7:e36890), blunts IFNα and iNOS expression (Kovats S. Cell Immunol. 2015; 294:63-9), and suppresses T-cell mediated auto-immunity-critical regulators of left ventricular remodeling and heart failure (Aggelakopoulou et al. J Immunol. 2016; 196:4947-56). This heretofore-unappreciated role of ERβ activation on expansion and pro-inflammatory switching of immune cells raises the possibility of an approach to immunomodulation that specifically reverses this pathological and tissue-injurious phenotypic switching of immune cells during the progression of chronic heart failure.

Epidemiologic studies have shown that pre-menopausal women are more protected against cardiovascular disease (CVD) as compared to post-menopausal women (Hayward et al. Cardiovasc Res. 2000; 46:28-49). Moreover, the incidence is much lower in women, in general, and is delayed by 10 years when compared with age-matched males (Wake et al. Recent Pat Cardiovasc Drug Discov. 2009; 4:234-40). Males, as compared to females, have also been shown to have significantly more pathological cardiac remodeling with accentuated activation of fibrotic and inflammatory genes (Wake et al. Recent Pat Cardiovasc Drug Discov. 2009; 4:234-40), suggesting a role for estrogen receptors (ERs) in mediating cardiovascular disease related inflammation.

Whereas, ERα levels are associated with maladaptive cardiac remodeling in human heart failure patients (Mahmoodzadeh et al. Faseb J. 2006; 20:926-34), ERβ overexpression improves heart function and survival by reducing cardiac fibrosis (Pedram et al. Mol Cell Endocrinol. 2016; 434:57-68). ERα mediated signaling regulates the production of type I IFNs in macrophages and dendritic cells and ERα^{-/-} immune cells produce significantly lower amounts of pro-inflammatory cytokines such as IL-6, IL-23, IL-12 and IL-10 (Kovats S. Cell Immunol. 2015; 294:63-9). Notably, all of these cytokines have been shown to be increased in heart failure patients (Dubnika et al. Cytokine Growth Factor Rev. 2018). Activation of ERβ, on the other hand, has been shown to inhibit TGFβ synthesis and myofibroblastic transition from the fibroblasts and blunts the fibrotic events induced by Angiotensin II and endothelin-1 (Pedram et al. Mol Cell Endocrinol. 2016; 434:57-68). Estradiol, via ERβ stimulation, also inhibits TNFα mediated activation of NF-kB (Xing et al. PLoS One. 2012; 7:e36890) and inhibits iNOS production in peritoneal macrophages (Xiu-li et al. Mol Immunol. 2009; 46:2413-8). A recent study has also shown that ERβ mediated signaling in CD4+ T-cells can be important for the suppression of autoimmune reactions in multiple sclerosis (Aggelakopoulou et al. J Immunol. 2016; 196:4947-56). Although, these studies provide preliminary evidence for the role of ERβ in regulating immune responses, whether and to what extent this happens in immune activation during heart failure is not known. In several published studies (Ismahil et al. Circ Res. 2014; 114(2); 266-82; Nahrendorf et al. J Exp Med. 2007; 204:3037-47; Bansal et al. Circ Heart Fail. 2017; 10:e003688; Kingery et al. Basic Res Cardiol. 2017; 112:19; Yang et al. Circulation. 2006; 114:2056-64), it has been shown that immune activation acutely after myocardial infarction (1-10 days) is protective in nature and mediates tissue repair. It has also been shown that chronic heart failure (4 to 8 weeks post-myocardial infarction) is associated with a 2nd wave of immune activation (Ismahil et al. Circ Res. 2014; 114(2); 266-82; Bansal et al. Circ Heart Fail. 2017; 10:e003688; Kingery et al. Basic Res Cardiol. 2017; 112:19), during which they undergo a phenotypic pro-inflammatory switch associated with increased TNFα and iNOS production and this phase coincides with the highest rates of fibrosis, hypertrophy, and maladaptive left ventricular remodeling. However, the molecular mechanisms that aid in this transition and the conditions that trigger immune cells to become pathological are not clearly defined.

Hence, it is hypothesized that selective ERβ activation can: inhibit TNFα mediated NF-kB translocation, ameliorate left ventricular remodeling, and improve cardiac function by deterring the activation of pathological immune cells. It is further suggested that ERβ stimulation can represent a cellular target for therapeutic immunomodulation in heart failure.

Proposed studies will identify therapeutic indications for Compound 1 as a selective immune-modulator to curb sustained immune activation associated with chronic heart failure. ERβ activation on immune cells can potentially reverse the proinflammatory phenotype of immune cells which in turn can prevent pathological left ventricular remodeling and progressive cardiac dysfunction in heart failure patients. Despite widespread understanding that heart failure is a state of chronic inflammation; no large-scale safe immunomodulatory therapies for heart failure have yet been successfully translated to clinical practice. To date, attempts at therapeutic immunomodulation in heart failure have primarily focused on protein mediators such as inflammatory cytokines. Proposed herein is a paradigm for immune-modulation in which leukocytes primed against the heart are targeted to reverse their proinflammatory phenotype using a selective ERβ agonist rather than cytokine mediators.

Aim 1: To delineate spatio-temporal changes in ERα and ERβ expression on immune cells during ischemic heart failure.

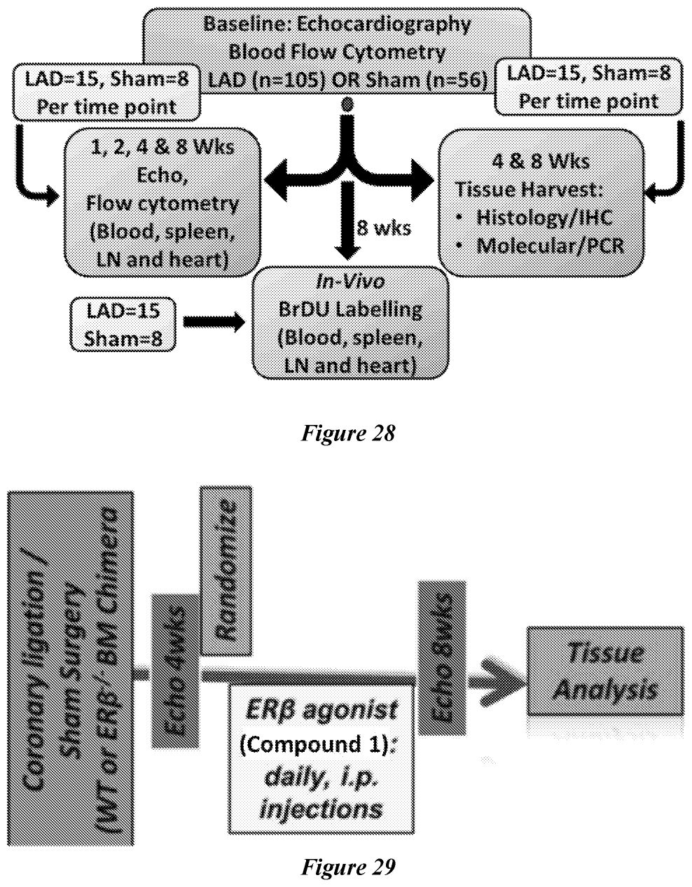

Rationale. Recent studies have defined the sequence of events that lead to activation, and infiltration of systemic and tissue-resident innate and adaptive immune cells during the early (Nahrendorf et al. J Exp Med. 2007; 204:3037-47) and late stages of post-myocardial infarction left ventricular remodeling (Ismahil et al. Circ Res. 2014; 114(2); 266-82; Epelman et al. Immunity. 2014; 40:91-104; Heidt et al. Circ Res. 2014; 115:284-95; Lavine et al. Proc Natl Acad Sci USA. 2014; 111:16029-34). Several studies have also shown that ERs are constitutively expressed on the immune cells of myeloid (monocytes/macrophages) and lymphoid origin (B and T lymphocytes) (Kovats S. Cell Immunol. 2015; 294:63-9; Aggelakopoulou et al. J Immunol. 2016; 196:4947-56). However, detailed spatio-temporal alterations of ERs on the immune cell populations during ischemic heart failure has not been performed. Hence, the working hypothesis herein is that immune cells progressively shift the expression of ERs by reducing ERβ levels to effect persistent global inflammatory activation (e.g., heightened systemic TNF elaboration) during the evolution of chronic heart failure. As the spatiotemporal kinetics of this switch are unknown, in Aim 1 the activation, proliferation, and pro-inflammatory phenotype of immune cells (monocytes/macrophages and T-cells) in PB, spleen, mediastinal LNs and the heart at 1, 2, 4 and 8 weeks post-myocardial infarction will be characterized (FIG. 28). More specifically, ERα and ERβ expression will be profiled on monocytes/macrophages and CD4⁺ T-cells in the heart, blood, spleen, and mediastinal LNs by flow cytometry and/or immunohistochemistry at 1, 2, 4, and 8 weeks after coronary ligation in mice as compared with sham operated controls. TNFα and nuclear factor (NF)-κB p65 will be used to index pro-inflammatory signaling, and will also index immune cell proliferation. Using human heart failure samples, the expression of ERs on circulating immune cells in ambulatory patients with systolic heart failure versus matched non-failing controls will be profiled.

Aim 2: To establish the protective role of immune cell specific ERβ on left ventricular remodeling and chronic heart failure

Rationale. ERβ plays an obligatory role in the biology of immune cell by regulating their activation, and the expression of pro-inflammatory cytokines such as TNFα (Xing et al. PLoS One. 2012; 7:e36890) and iNOS (Xiu-li et al. Mol Immunol. 2009; 46:2413-8). Furthermore, ERβ activation has also been shown to suppress immune activation in auto-immune diseases (Aggelakopoulou et al. J Immunol. 2016; 196:4947-56). Since heart failure is also characterized by autoimmune reactions, treatment with a selective ERβ agonist can provide therapeutic benefits by inhibiting pathological left ventricular remodeling mediated by auto-immune reactions. Therefore, Compound 1 will be injected (i.p.) daily in the heart failure mice from 4 to 8 weeks post-myocardial infarction (FIG. 29). This time-point was chosen as previous studies have shown that immune cells undergo a pathological phenotypic switch at around 4 weeks post-myocardial infarction (Bansal et al. Circ Heart Fail. 2017; 10:e003688). Cardiac function will be measured before (4 weeks post-myocardial infarction) and after the treatment (8 weeks post-myocardial infarction) using echocardiography and monocytes/macrophages and t-cells will be profiled using flow cytometry. The cardiac function will be evaluated before (4 weeks post-myocardial infarction) and after the treatment (8 weeks post-myocardial infarction), changes in systemic/cardiac immune cell profiles will be evaluated, and left ventricular remodeling (hypertrophy, apoptosis, fibrosis, and capillary rarefaction) during chronic heart failure will be evaluated. Several parameters of left ventricular remodeling, such as myocyte hypertrophy (wheat-germ agglutinin staining), fibrosis (masson trichrome staining), myocyte apoptosis (TUNEL staining) and capillary rarefaction (isolectin staining), and ERβ signaling in isolated cardiac and splenic immune cells, will also be evaluated.

Since ERβ is also expressed on myocytes, these studies will be repeated in bone-marrow (BM) chimera mice. Wild type mice will be lethally irradiated and reconstituted with the BM from the ERβ^{-/-} mice to specifically deplete ERβ from the immune cells. It is expected that the cardio-protective effects of ERβ activation will not be observed in these mice, which can definitively prove an obligatory role of ERβ receptor signaling in immune cell activation during chronic heart failure.

These studies investigating the importance of ERβ on immune cells in the pathogenesis of left ventricular remodeling and chronic ischemic heart failure will thereby further the understanding of the cellular basis for inflammation in this disease. Moreover, by providing direct evidence for the protective role of ERβ in inhibiting cardiac specific immune cell activation, therapeutic indications for ERβ agonist for immunomodulation in heart failure can be identified.

### Example 3

Methods. Animal studies were approved by the Institutional Animal Care and Use Committee at the Ohio State University and were done in accordance with the NIH Guide for the care and Use of Laboratory Animals (DHHS publication No. 85-23, revised 1996). All mice had free access to food and water ad-libitum and a total of 138 mice were used for all the studies.

Mouse Model, Surgical Protocol and Drug Treatment. Male, 10 to 12-week-old C57BL/6 mice (Jackson Laboratories, stock #000664) underwent left thoracotomy followed by permanent left coronary artery ligation to induce MI and ischemic HF (n=80) or sham surgery (n=28), as described previously. At 8 weeks post MI, peripheral blood was collected from the facial vein and the mice were euthanized by cervical dislocation. Hearts and spleens were collected, weighed, and processed either for mononuclear cell isolation or histological analysis. Tibia length was measured to normalize all gravimetric data.

A 20-fold stock solution of compound 1 was prepared in DMSO and stored at −20° C. At the time of dosing, the drug was diluted using an equal volume of tween 20 and saline maintaining a ratio of 5:5:90 (DMSO:Tween 20:saline). All mice were weighed every day and the drug was administered at 60 mg/kg dose (200 μL per 25 g body weight) via gavage.

Echocardiography. Echocardiography was conducted under 1-1.5% isoflurane anesthesia using a VisualSonics Vevo 3100 and body temperature was maintained using an adjustable heated rail system (Vevo Imaging Station) and RMV707B scanhead as previously described.

Immune Cell Isolation and Fixation. Immune cells were isolated from the spleens, blood, and hearts, as described previously. Briefly, spleens were triturated using the plunger of a 3-mL syringe in sterile PBS supplemented with 2% BSA and 2 mM EDTA to release all splenocytes and were filtered through 40 μm cell strainers to remove connective tissue. Peripheral blood (100 μL) was collected from cheek veins in BD microtainer tubes containing EDTA, and RBCs were lysed using lysis buffer (eBioScience). Hearts were finely minced, digested using collagenase II (1 mg/mL) to obtain single cell suspensions, and filtered through 40 μm cell strainers. Cells from the tissues were pelleted by centrifugation at 500 g, re-suspended in 100 μL PBS supplemented with 2% BSA and 2 mM EDTA, fixed using 100 μL of 1% w/v PFA, and stored at 4° C. for flow cytometric staining.

T-cell Proliferation Assays. Splenic CD4+ T cells were magnetically purified using automated RoboSep™ cell separation system from Stemcell technologies and MojoSort Mouse CD4⁺ T-cell isolation kit (BioLegend), as per manufacturer's instructions. Purified live cells were counted using trypan blue exclusion assay, reconstituted at a concentration of 1×10⁶ cells/mL in sterile PBS and were incubated at 37° C. in the dark with an equal volume of 4 μM Tag-it Violet™ (BioLegend) for 10 min. Excess dye was quenched by adding complete RPMI media (supplemented with 10% charcoal stripped FBS to endogenous hormones and 1% P/S/G) (Gibco) at five times the volume of the dye solution used for staining, and incubating for 5 min on ice. Labeled cells were pelleted by centrifugation at 500 g for 5 min and re-suspended in complete RPMI media at a concentration of 1×10⁶ cells/mL for all assays.

Flat bottom 96-well plates (FisherSci) were incubated with 50 μL of a 4.5 mg/mL solution of Anti-Hamster IgG (MilliporeSigma) at room temperature. After 1 h, wells were washed with sterile PBS to remove excess antibody, and were coated with 50 μL of rat anti-mouse CD3 antibody (BioLegend) at a concentration of 2 μg/mL for 1 h at room temperature. Non-stimulated control wells were incubated only with sterile PBS. All the wells were washed with sterile PBS to remove excess antibody followed by plating of 1×10⁵ Tag-it Violet™ dye labeled CD4⁺ T-cells in each well. Co-stimulation was effected by adding 100 μL of complete RPMI media containing 4 pg/mL of Rat anti-mouse CD28 antibody (BioLegend) while 100 μL of complete RPMI media (without anti-CD28 antibody) was added to non-stimulated wells. Cells were incubated for 72 h at 37° C. and 5% CO₂ and dye dilution with each successive cell division was measured either by Becton Dickinson LSRFortessa or NL3000 Northern Lights (Cytek®) flow cytometer.

Flow Cytometric Staining. Detailed protocol for cell staining has been described previously. Briefly, cell pellets were re-suspended, aliquoted into flow tubes and incubated on ice with a cocktail of extracellular rat anti-mouse antibodies for 45 min. Cells were washed with PBS supplemented with 2% BSA and 2 mM EDTA and fixed using 1% PFA. For intracellular staining, cells were permeabilized using 0.5% v/v tween-20 and incubated with a cocktail of antibodies (on ice) for 45 min. Anti-mouse CD4 SB600/PE-Cy7, Foxp3-APC, IFNγ eFluor 450, Ly6C eFluor 450 and ERβ antibodies were from ThermoFisher Scientific; CD8 APC-Cy7, CD11b APC-Cy7, and CD19 PerCP-Cy5.5 antibodies were from Tonbo Biosciences; IL-17 AF700, TNFα FITC, CD11b-APC, CD69 PerCP, Ly6G PE and CD69 AF700 antibodies were from Biolegend and ERα PE antibody was from Abcam.

Hypertrophy (WGA) Staining. Formalin-fixed, paraffin-embedded hearts were sectioned (5 μm thickness), deparaffinized, rehydrated, and stained as described previously. Masson's trichrome staining was used to quantify tissue fibrosis while Alexa Fluor 488-conjugated wheat germ agglutinin (ThermoFisher Scientific) was used to label cellular membranes. Myocyte area was quantified in the remote zone of failing hearts from 3 to 4 high-power fields per section.

Statistical Analysis. All data are shown as mean±SD. Unpaired student's ‘T’ test with equal or unequal variance was used to compare 2-groups while 1-way or 2-way Anova with Tukey's post-hoc test was used for comparing more than 2 groups. In some cases, 1-way Anova with correction for multiple comparisons by controlling the false discovery rate was used. GraphPad Prism version 9.0 was used for all statistical analyses and a P value of <0.05 was considered significant.

ERα Signaling is Upregulated in CD4⁺ T-Cells During Ischemic HF

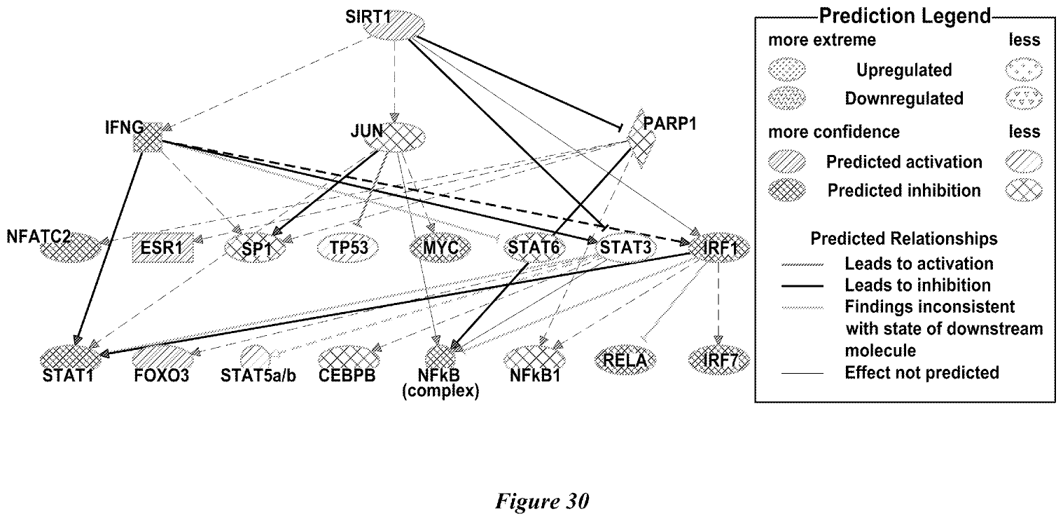

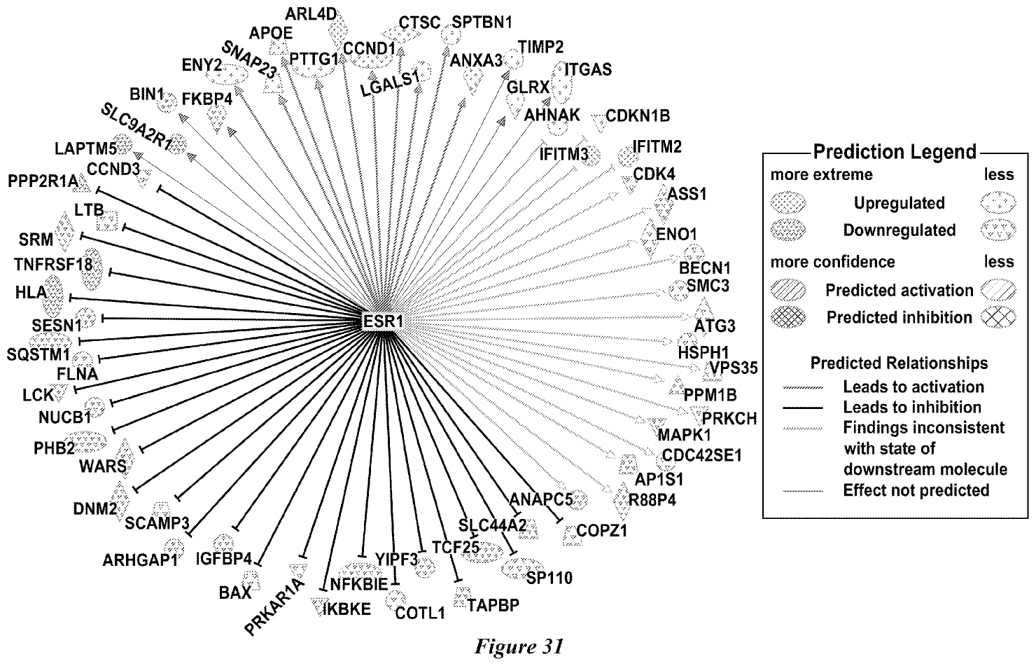

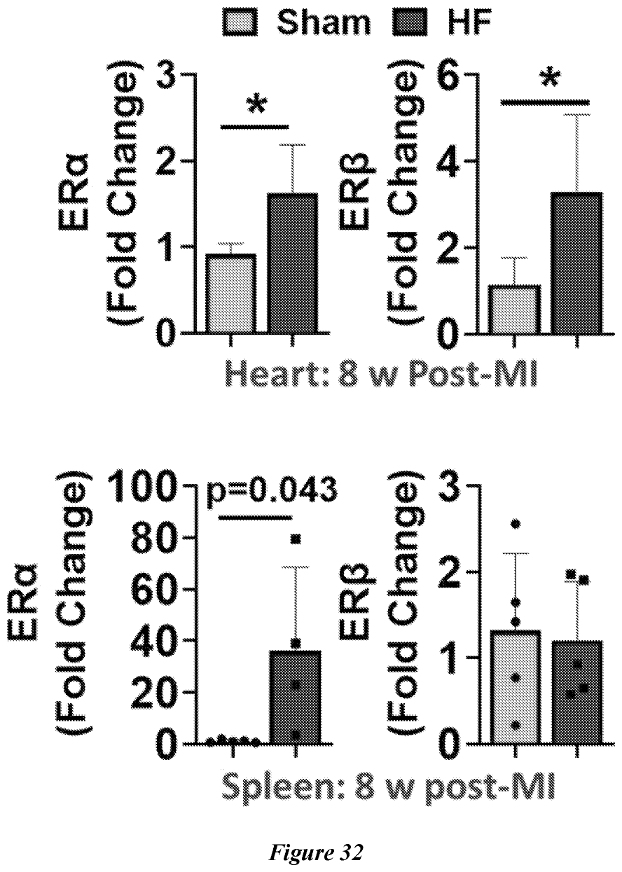

In previous studies, it was shown that, in contrast to their wound-healing properties during myocardial infarction (MI), CD4+ T-cells undergo a pathological phenotypic shift during chronic heart failure (HF), and accentuate left ventricular (LV) remodeling and cardiac dysfunction. Therefore, to identify potential phenotypic switches, CD4+ T-cells (150-300) were sorted from the failing hearts & the mediastinal lymph nodes of the male mice at 8 wks post-MI and limited cell RNA-sequencing was conducted (FIG. 30). IPA analysis of the differentially expressed genes showed that downstream of SIRT1, ESR1 (ERα) signaling was significantly upregulated in cardiac CD4⁺ T-cells as compared to the mediastinal lymph nodes of HF mice and several genes downstream of ESR1 signaling were altered (FIG. 31). This was highly interesting as these T cells were sorted from male failing hearts. To further validate these findings, the gene expression of ERα and ERβ in the remote zone-LV and splenic mononuclear cells from the HF mice was measured. As shown in FIG. 32, while both ERα and ERβ were increased in the hearts, only ERα was increased in the splenic immune cells.

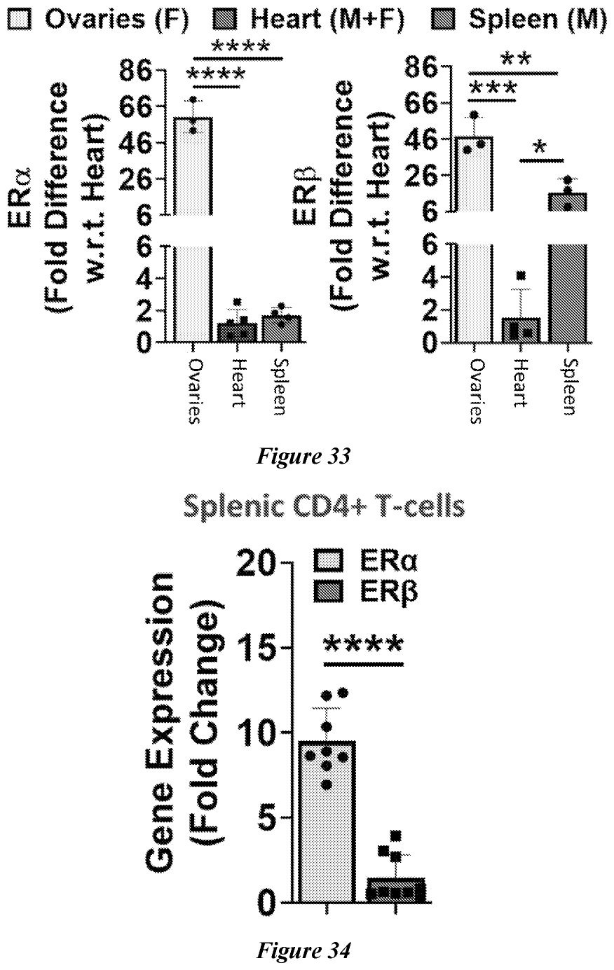

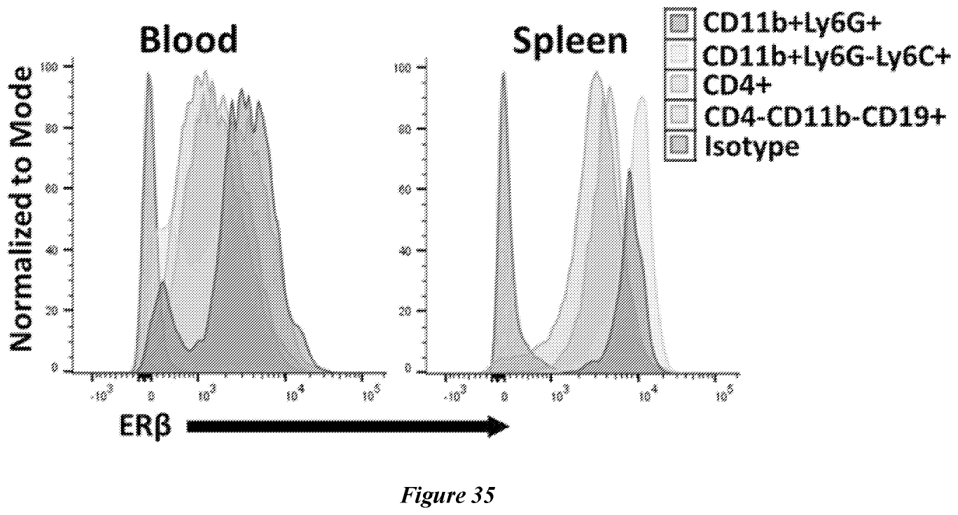

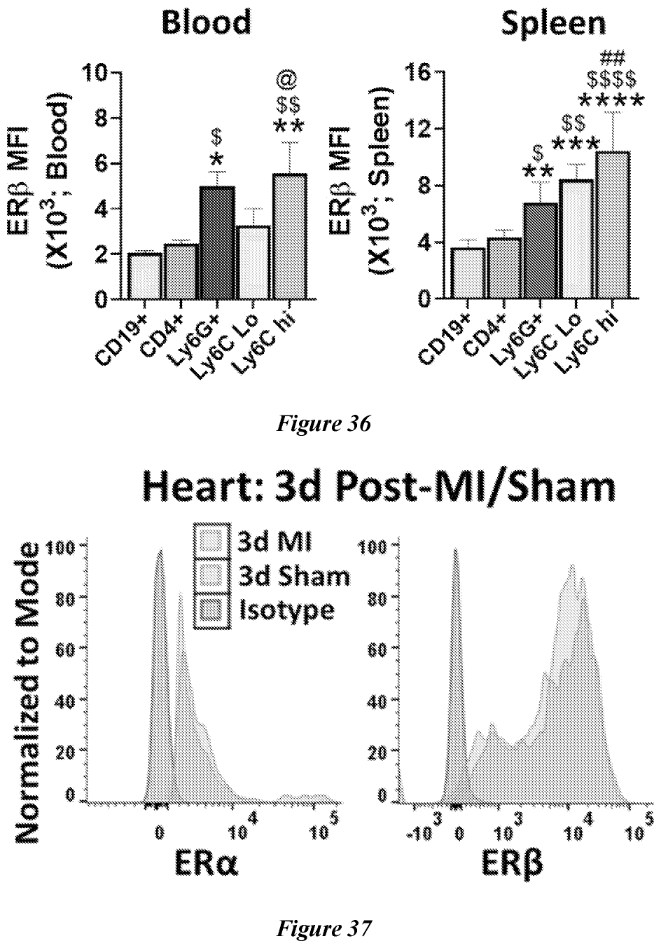

Several studies in other autoimmune diseases, such as multiple sclerosis (MS) and experimental autoimmune encephalomyelitis (EAE), have shown the role of estrogen receptors (ERβ) in modulating T-cell activity. However, these studies are mostly done using ovariectomized female mice and very limited work has been done in male mice. Therefore, steady-state expression of ERα and ERβ was measured in the ovaries (as a positive control), hearts of male and female mice, and spleens of naïve male mice. As shown in FIG. 33, while ERα expression in the hearts and spleens was comparable; ERβ expression was ˜11-fold higher in the spleen as compared to the heart. Splenic CD4⁺ T-cells from the naïve male mice were then magnetically sorted and ER expression in them was measured. Interestingly, ERβ expression in splenic CD4+ T-cells was 10-fold lower as compared to ERα (FIG. 34), suggesting that ERα signaling is predominant in CD4+ T-cells and that other splenic immune cells express significantly higher levels of ERβ as compared to CD4+ T-cells. This was consistent with other studies showing highest expression of ERα in T-cells isolated from the human PBMCs. Further analysis of ERβ expression in different circulating and splenic immune cells showed this to be the case, as cells of the myeloid origin (such as Ly6G+ neutrophils & Ly6C+ monocytes) expressed much higher ERβ as compared to CD4+ T-cells, while CD19+ B-lymphocytes had comparable expression (FIG. 35 and FIG. 36).

ERα is Temporally Modulated while ERβ Expression is Spatially Altered in CD4⁺ T-Cells During Ischemic HF.

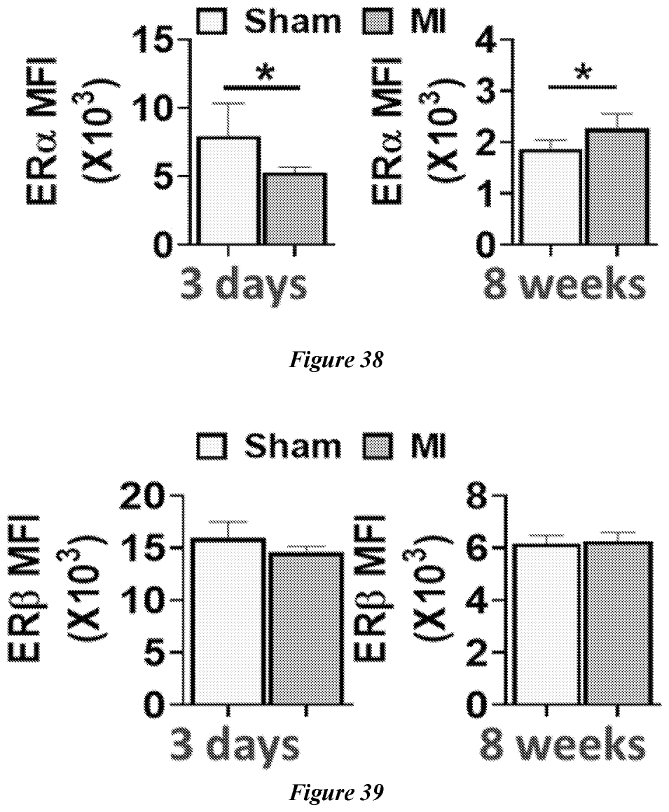

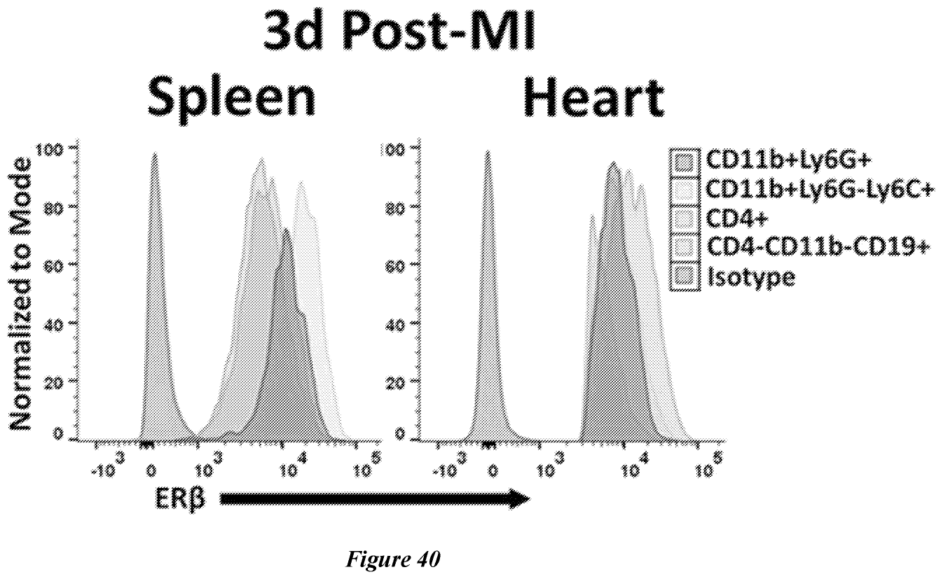

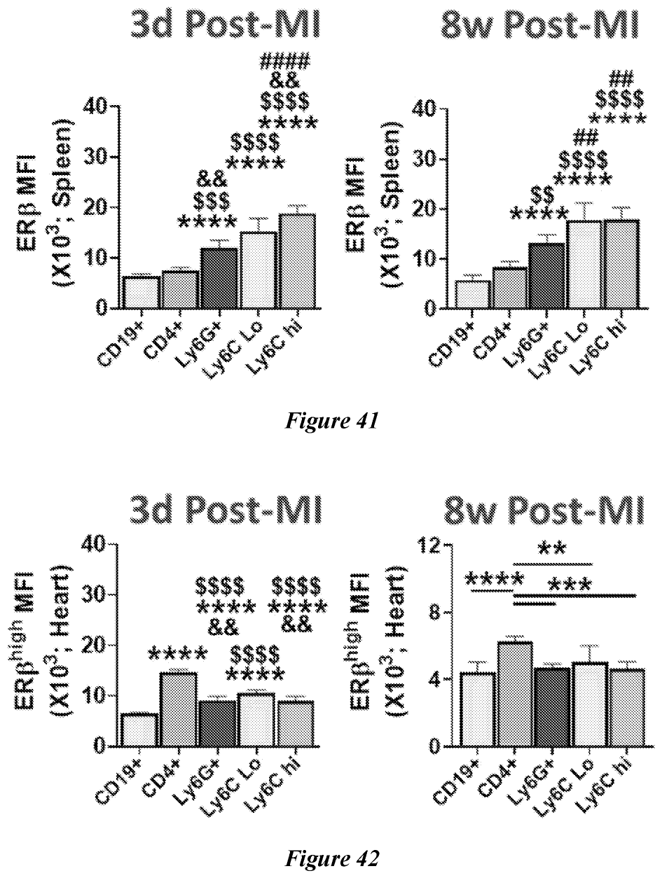

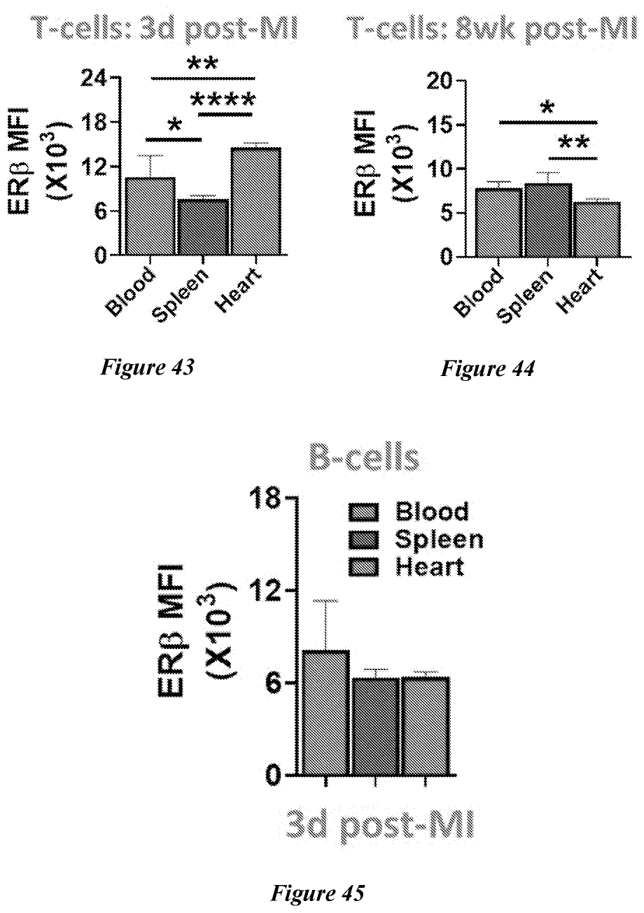

Epidemiological (in premenopausal women) and preclinical studies indicate protective effects of ER signaling in cardiovascular diseases. However, given the pathological role of T-cells identified in previous studies, it is not known what role, if any, ER signaling plays in modulating CD4⁺ T-cell polarization/activation in the context of MI and HF. Since 3 days (d) post-MI marks the peak inflammatory response and 8 weeks (w) post-MI exhibits significant LV remodeling with pathological transitioning of CD4⁺ T-cells, ERα and ERβ expression in CD4⁺ T-cells was measured at these time-points. As shown in FIG. 37 and FIG. 38, ERα expression was significantly decreased at 3 d post-MI, but was significantly increased at 8 w, which was consistent with lc-RNA seq data showing the activation of ERα pathway at this time-point. ERβ expression, on the other hand, did not change at either of the time-points when compared with the sham mice (FIG. 39). Upon further analysis of spatial changes in ERβ expression, it was found that, despite low ERβ expression in spleen (FIG. 40 and FIG. 41), CD4+ T-cells infiltrating into the failing hearts had higher expression of ERβ as compared to other cells of myeloid, such as Ly6G+ neutrophils or Ly6C⁺ monocytes/macrophages, or lymphoid origin, such as CD19⁺ B-cells (FIG. 42). Moreover, at 3d post-MI, ERβ expression in cardiac CD4⁺ T-cells was much higher as compared to circulating or splenic T-cells, and was significantly lower at 8 weeks post-MI (FIG. 43 and FIG. 44). These changes were not due to differences in tissue background or differential non-specific tissue binding between splenic or cardiac single cell preparations, as B-lymphocytes (comparable in size and ERβ expression in the naive mice (FIG. 36)) did not exhibit this change in ERβ expression even at 3d post-MI (FIG. 45), the peak of acute inflammatory response post-MI. These data show that: 1) ERα expression is temporally regulated and it's high levels coincide with pathological transitioning of T-cells, 2) ERβ expression is significantly and sustainably increased in cardiac CD4⁺ T-cells when compared with other immune cells post-ischemic injury, 3) ERβ is spatially modulated, and its expression in CD4⁺ T-cells intensifies upon infiltration into the injured hearts at 3d post-MI but declines at 8 wks post-MI when compared with other tissues, and, last but not the least, 4) ERβ activation to constrain ERα signaling could be a potential target to blunt pathological transitioning of CD4⁺ T-cells.

ERβ Agonist Dose Dependently Inhibits Anti-CD3/CD28 Mediated T-Cell Proliferation

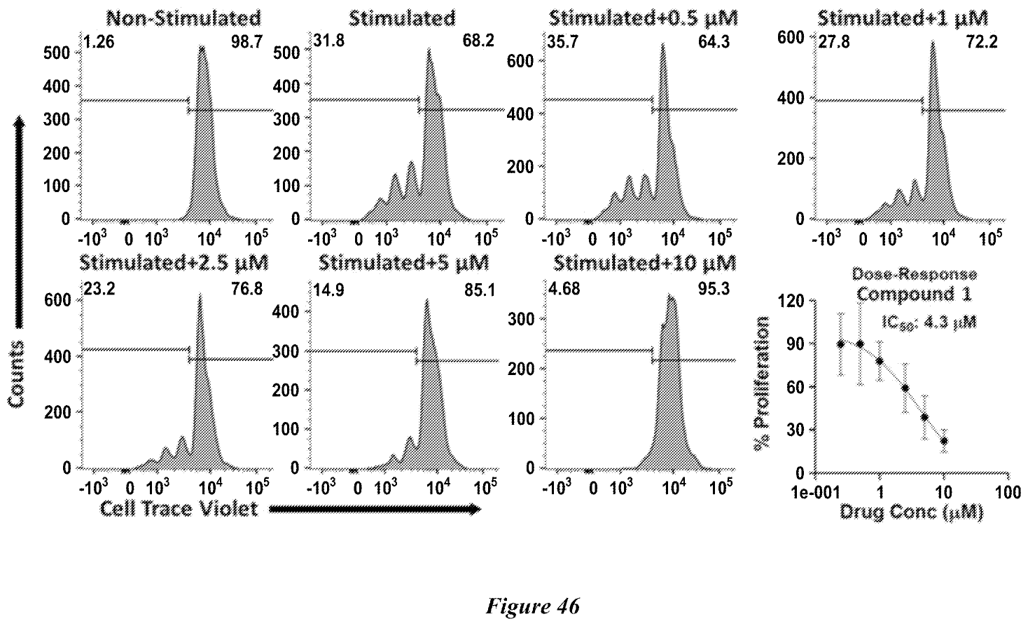

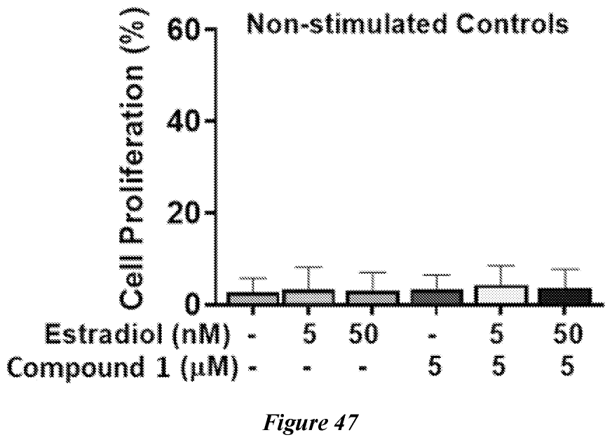

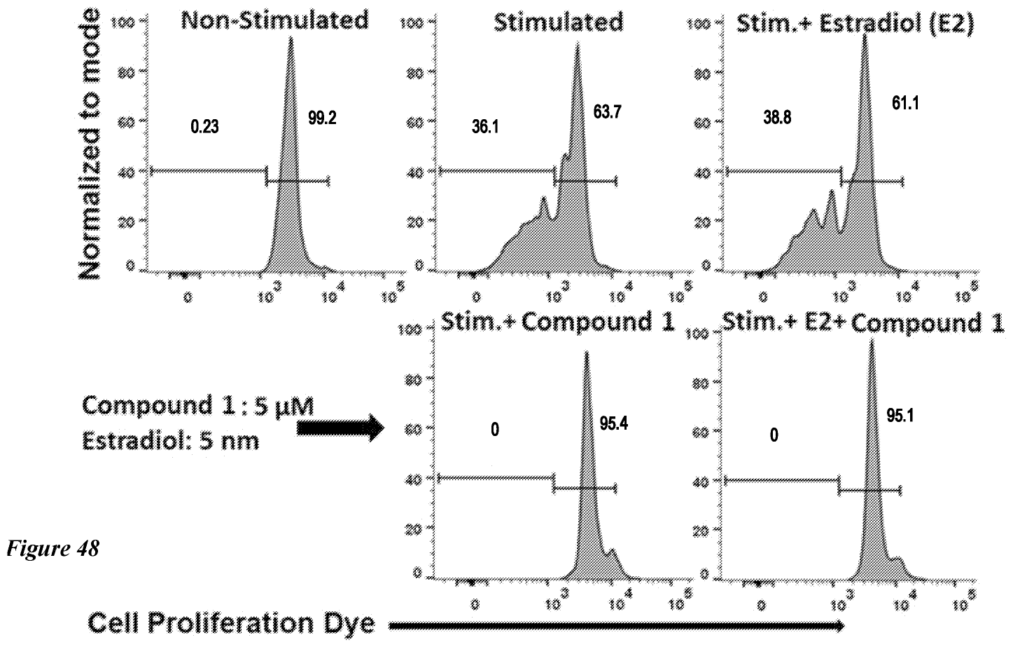

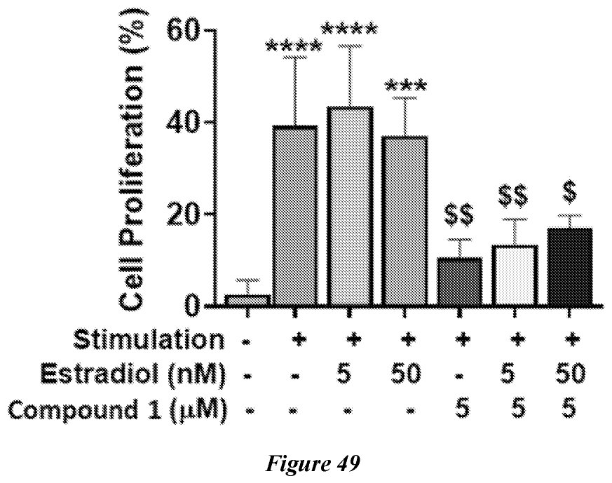

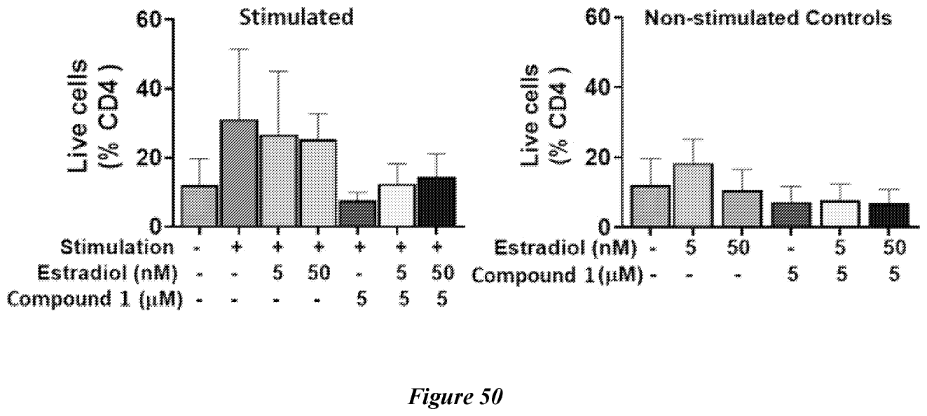

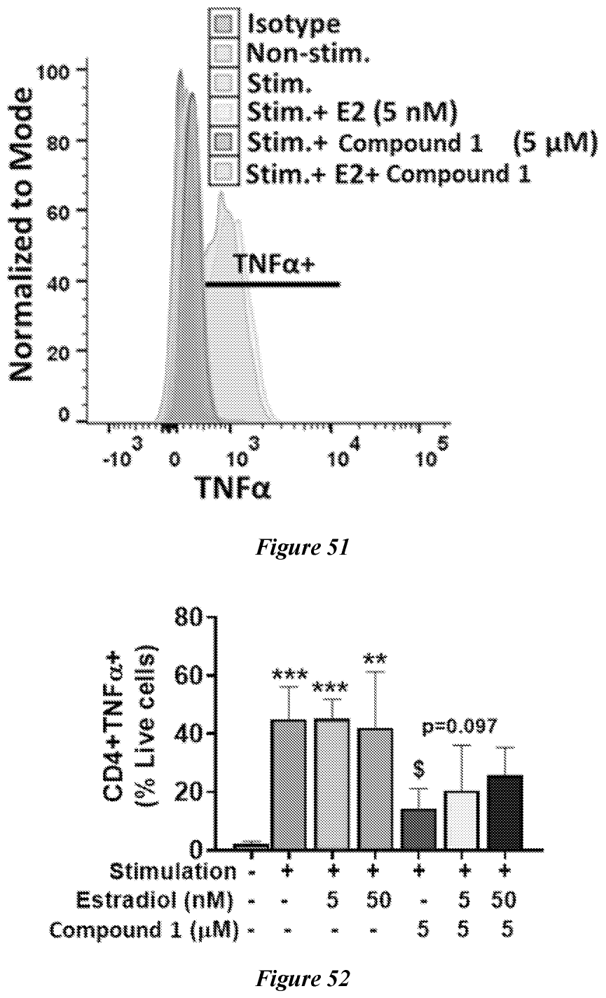

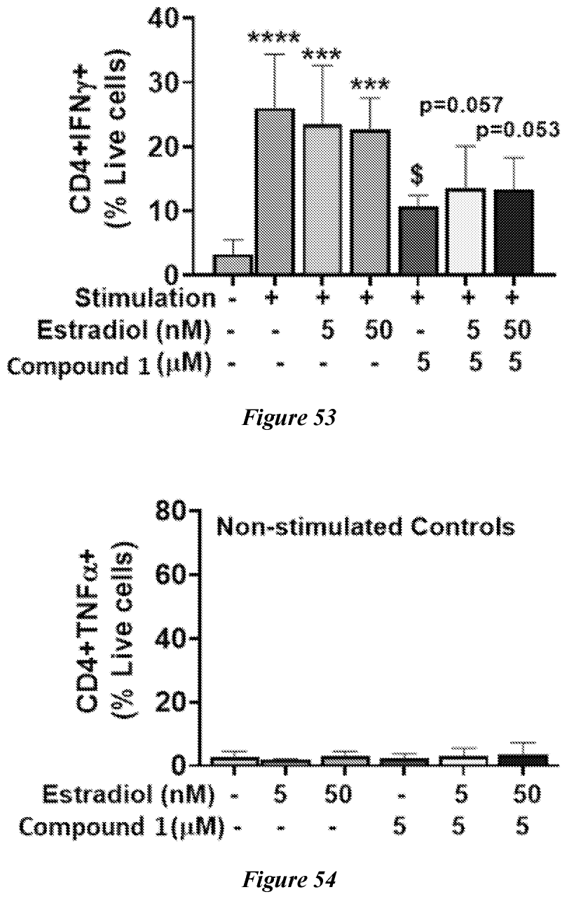

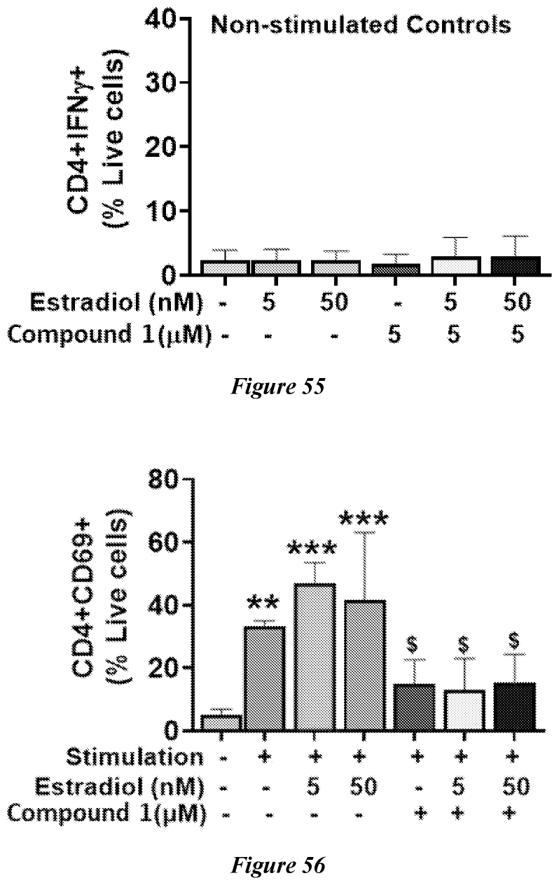

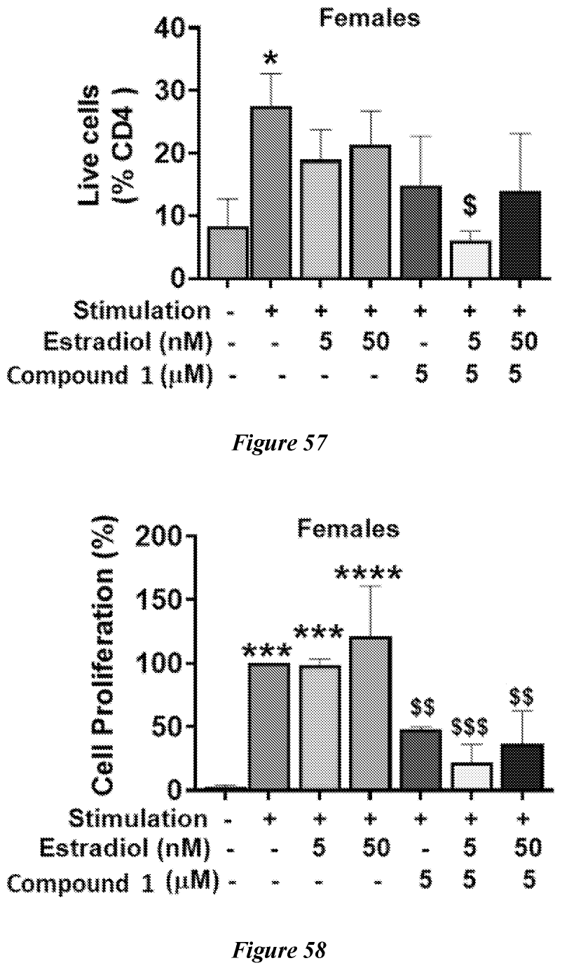

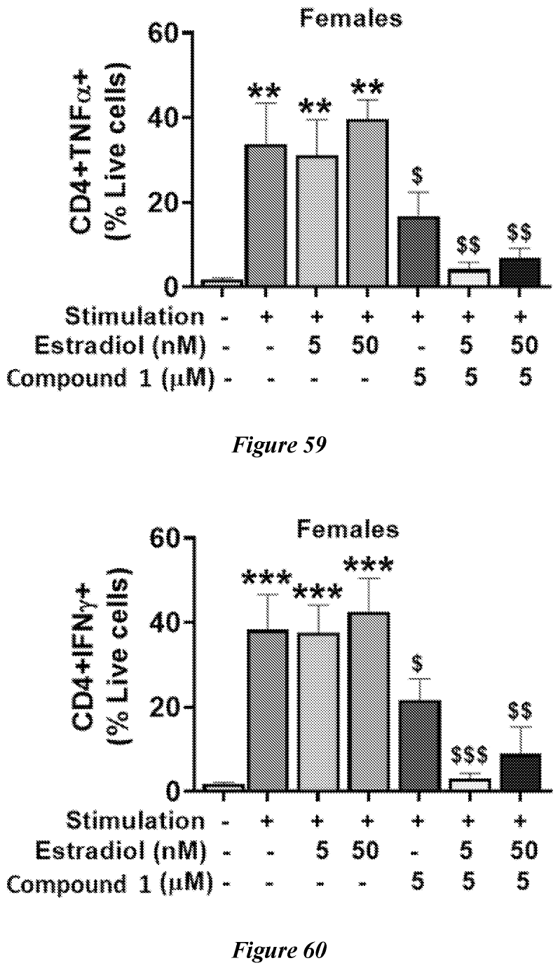

ERα signaling is antagonized by ERβ agonism. Thus, an ERβ agonist, Compound 1, a lipophilic and orally bioavailable compound with ˜200-fold selectivity for ERβ as compared to ERα was identified. This compound was tested for its ability to inhibit TCR (anti-CD3/CD28) mediated proliferation of CD4⁺ T-cells isolated from the spleens of naive male mice. As shown in FIG. 46, compound 1 dose-dependently inhibited TCR mediated T-cell proliferation with an IC₅₀ of 3.4 μM. Importantly, while the drug (5 μM) did not affect non-stimulated CD4⁺ T cells (FIG. 47), it significantly inhibited the proliferation of TCR activated CD4⁺ T-cells even in the presence of low and high estradiol concentrations (FIG. 48 and FIG. 49). When compared with non-stimulated T-cells, there was an increasing trend in the frequency of live cells upon TCR stimulation, presumably due to proliferation of live cells (FIG. 50). However, in the TCR stimulated T-cells treated with the 5 μM drug (FIG. 50 and FIG. 54), no change was seen in the frequency of live cells as compared to the non-stimulated cells, suggesting that the reduction in T-cell proliferation was not due to increased cell-death. Using this concentration, the effects of ERβ agonism on the expression of pro-inflammatory cytokines such as TNFα and IFN-γ was also tested. As compared to non-stimulated T-cells, TCR stimulation significantly increased TNFα (FIG. 51 and FIG. 52) and IFNγ (FIG. 53) expression both in the absence as well as in the presence of estradiol, which was significantly inhibited by the drug. There was no change in either the TNFα or IFNγ expression in the non-stimulated cells treated with the drug in the presence or absence of estradiol (FIG. 54 and FIG. 55), suggesting that the inhibition of cytokine expression was specific to T-cell activation and does not affect homeostatic expression of these cytokines. A significant reduction in the frequency of CD69 expressing CD4⁺ helper T-cells upon treatment with 5 μM compound 1 was also observed (FIG. 56), suggesting blunting of T-cell activation as well. Similar effects were observed with CD4+ T-cells isolated from the female mice (FIG. 57-FIG. 60), suggesting that, although the drug was specific for ERβ, the effects were not gender-specific, and T cells from both male and female mice were equally inhibited. This also indicates that the role of estradiol and ERs in T-cell activation is ubiquitous and TCR signaling through this pathway is not dependent upon the gender.

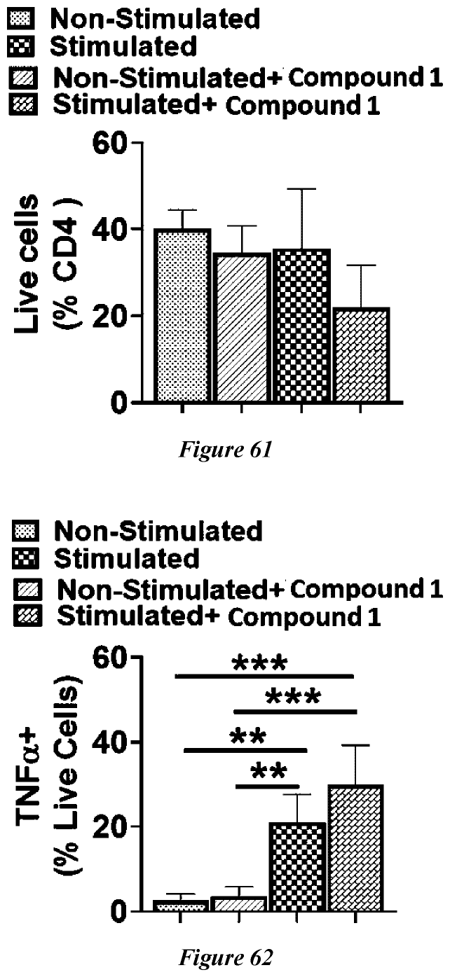

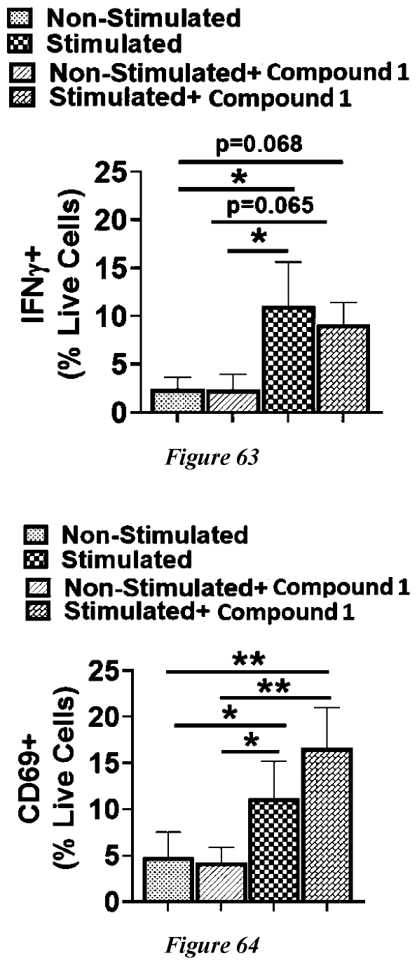

To determine if the inhibitory effects of compound 1 were against a specific activation mechanism or is non-specific in nature, the drug was also tested in the presence of PMA & Ionomycin (FIG. 61-FIG. 64). These agents activate T-cells by increasing intracellular Ca²⁺ and PKC activity, and bypasses TCR activation. PMA/Ionomycin treatment significantly increased the expression of pro-inflammatory cytokines, TNFα and IFNγ, and activation marker CD69 in T-cells (FIG. 61-FIG. 64). Interestingly, none of these pro-inflammatory and activation markers were inhibited by compound 1. These results suggest that compound 1 selectively inhibits TCR mediated T-cell proliferation while sparing other activation mechanisms.

Compound 1 Activates ERβ but Inhibits ERα Signaling

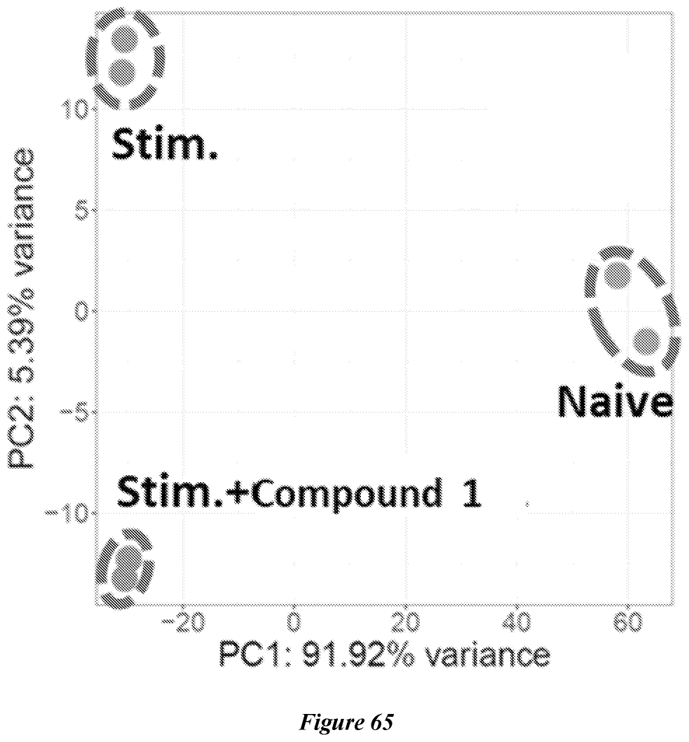

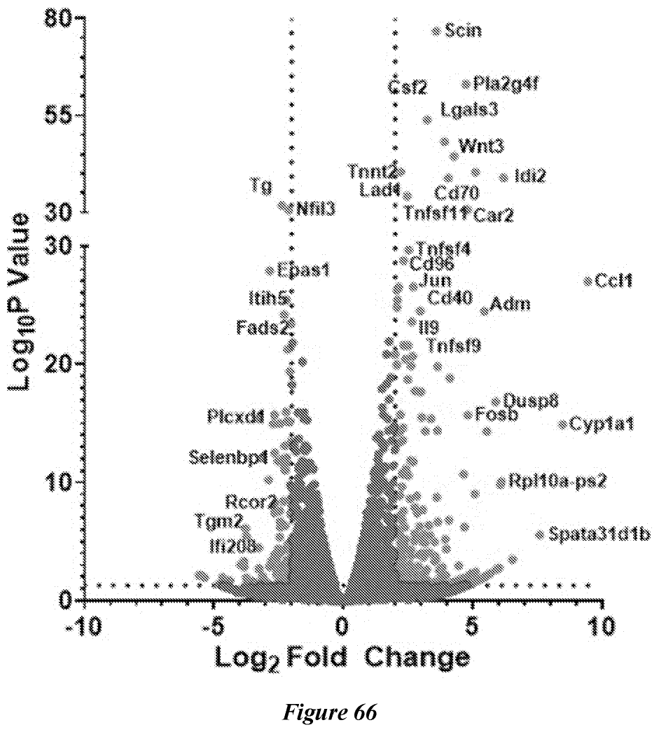

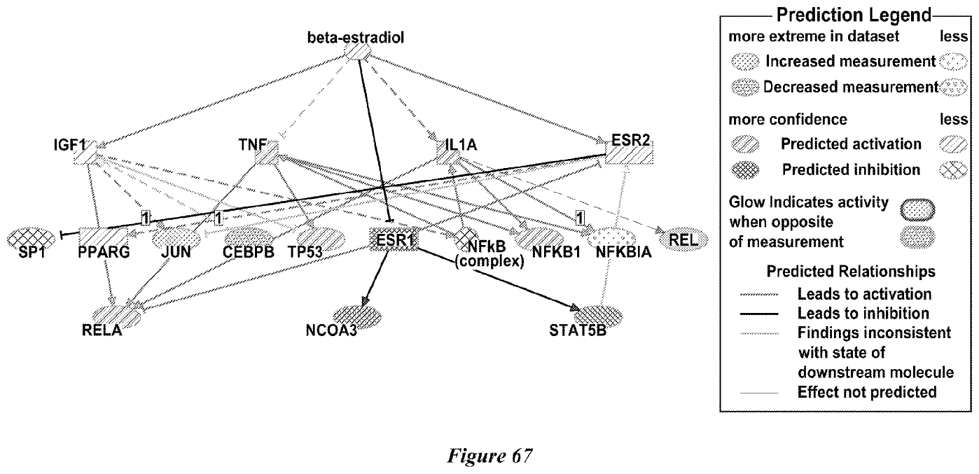

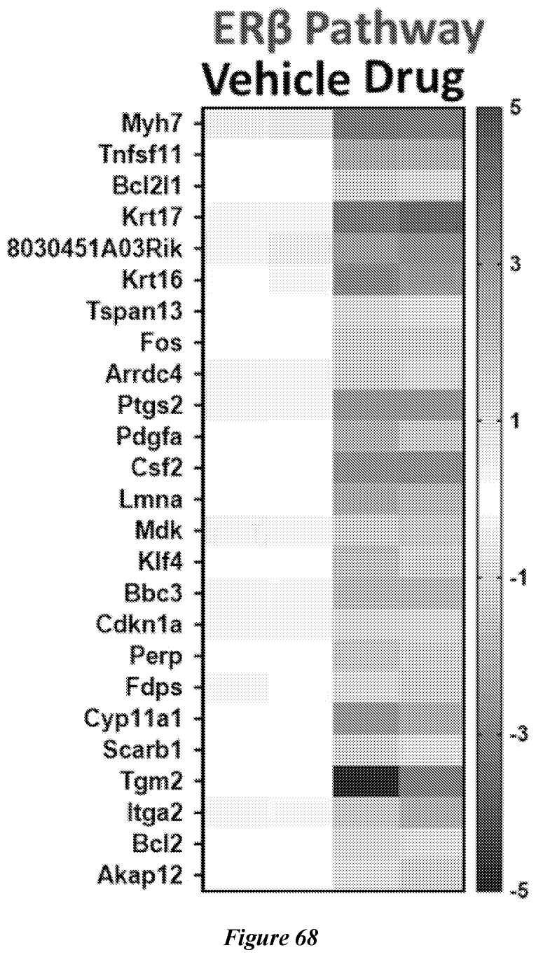

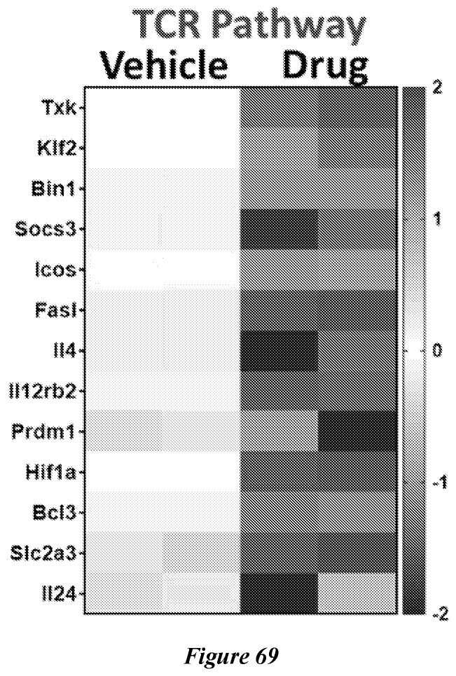

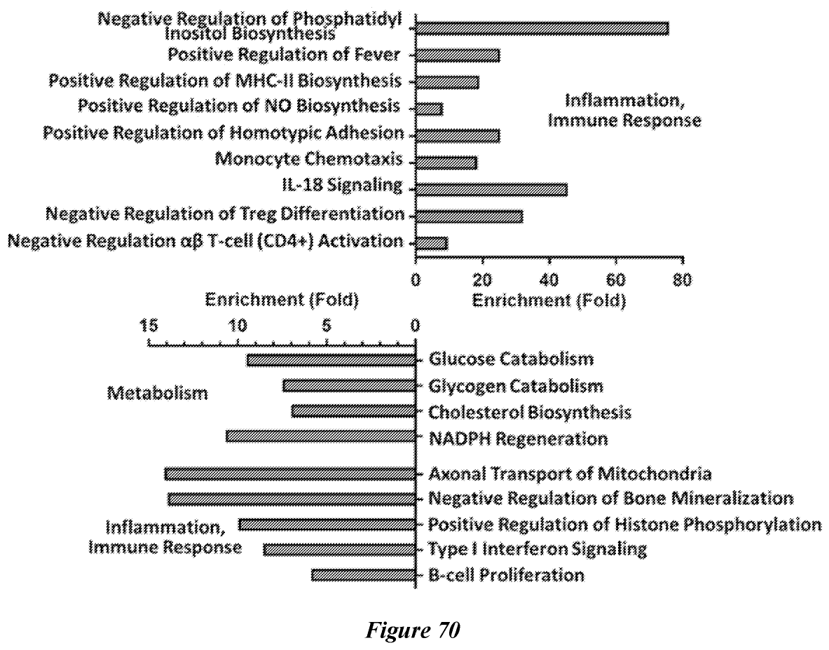

To further investigate the specificity of the drug, RNA was isolated from anti-CD3/CD28 stimulated CD4⁺ T-cells cultured with and without 5 μM drug. Principal component analysis (FIG. 65) showed that the RNA transcriptome of TCR activated CD4⁺ T-cells was significantly altered in the presence of 5 μM compound 1 as compared to the stimulated cells treated with the vehicle. Several genes were identified that were either downregulated or upregulated (>2-fold and p<0.01) in the presence of drug (FIG. 66). Several genes involved in ERβ pathway were upregulated while some were downregulated (FIG. 67 and FIG. 68), leading to an overall activation of this pathway with concomitant inhibition of ERα signaling. Downregulation of several genes involved in TCR activation was also observed, further supporting TCR specificity of compound 1. The effects of ERβ agonism on other pathways required for immune activation were further assessed. Analysis of upregulated and downregulated gene transcripts showed that several pathways involved in inflammation, immune activation, and metabolism were either inhibited or negatively-affected by the compound 1 (FIG. 69 and FIG. 70).

Compound 1 Treatment Specifically During Chronic HF Ameliorates Cardiac Remodeling

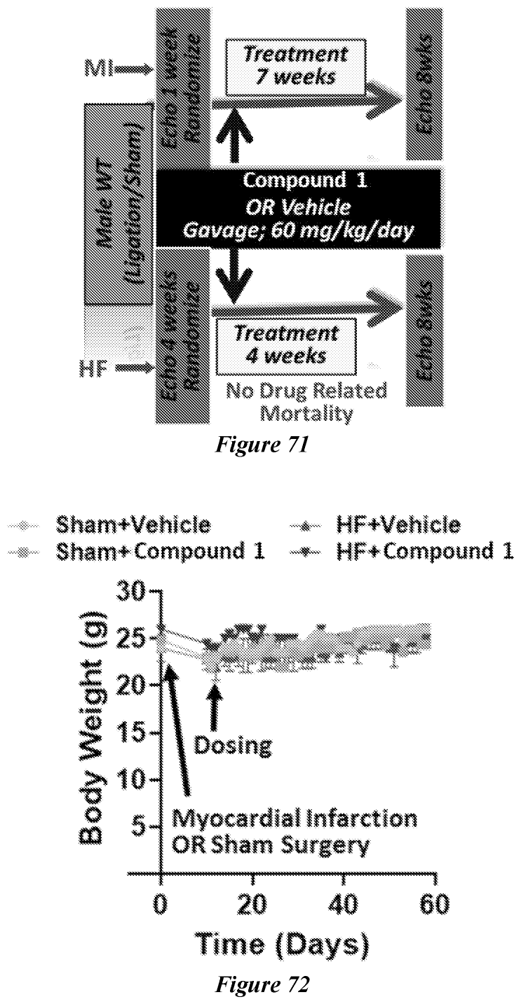

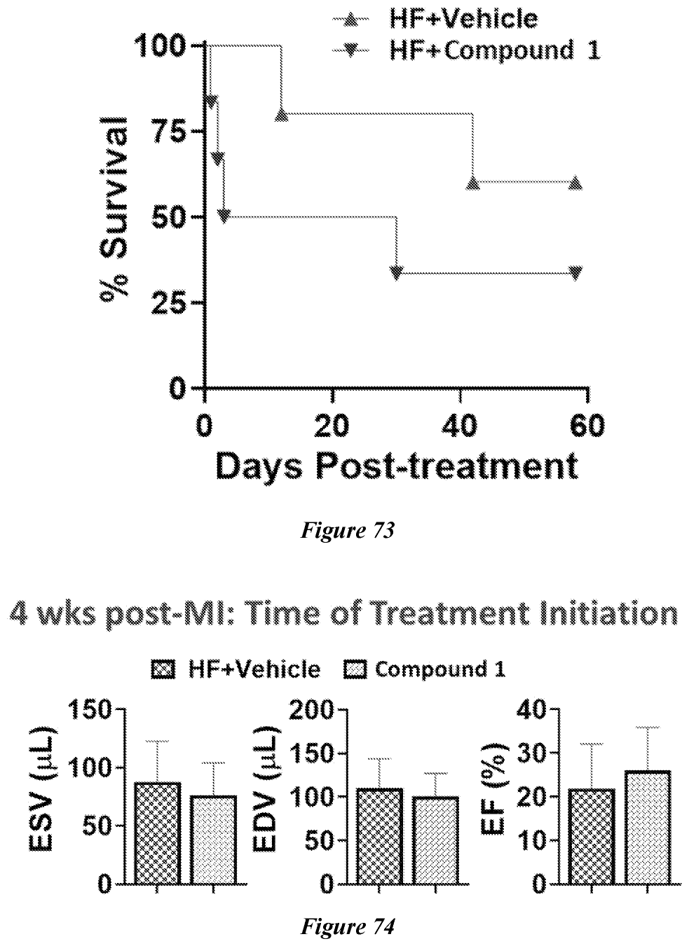

Considering that CD4+ T-cells are critical for would healing during initial 10-14 days post-MI but are pathological and dispensable during chronic HF, the efficacy of the drug at both phases was tested. For acute-MI, using echocardiography, akinetic area at 7 d post-MI was measured and mice were randomized to either receive vehicle or 60 mg/kg compound 1 (summarized in FIG. 71). Daily administration of the drug via gavage did not affect the body weight of infarcted or sham mice (FIG. 72), suggesting that the drug did not have any overt side-effects on rodent physiology. However, a significantly increased mortality was observed with drug treatment, as several mice (˜60%) died during the first week of treatment when compared with the vehicle control (15-20%) (FIG. 73). This, consistent with other studies, underscores the vital and protective role of CD4⁺ T-cells for adequate wound-healing post-MI.

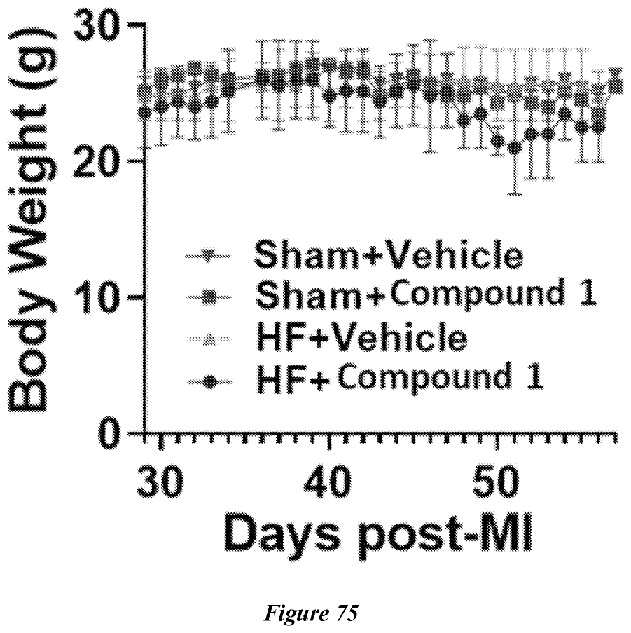

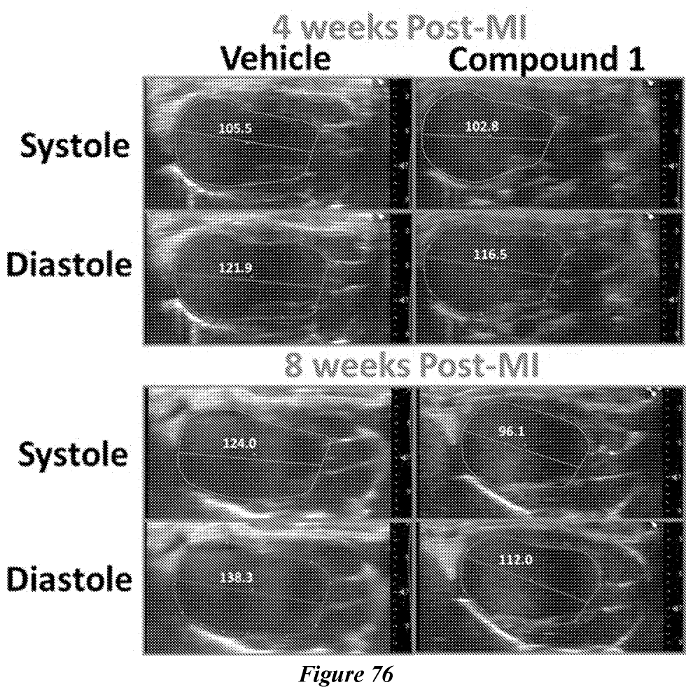

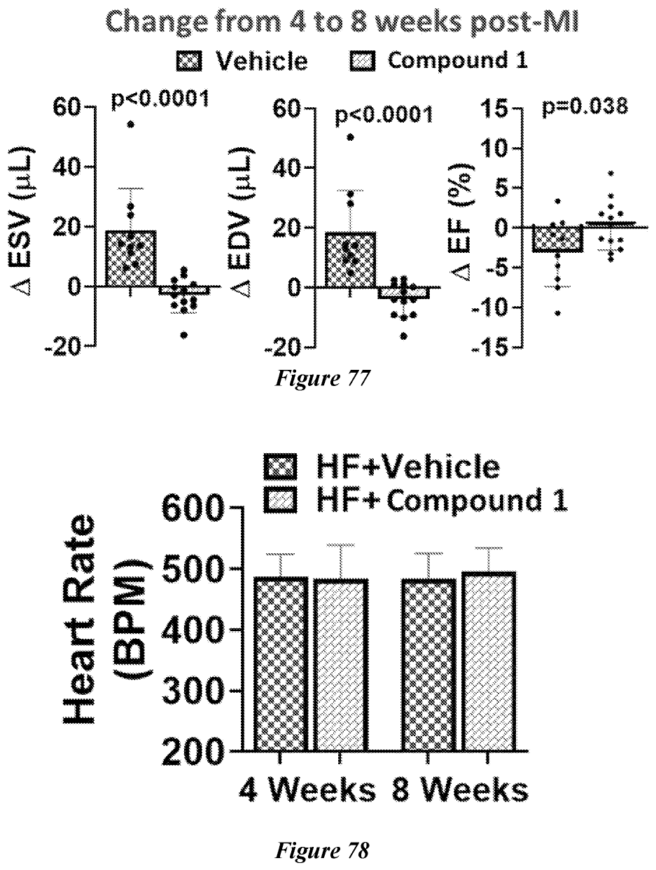

For chronic HF, cardiac function was measured at 4 w post-MI and all animals were randomized according to the degree of cardiac dysfunction (FIG. 74), reflected by end-systolic and end-diastolic volumes (ESV and EDV), and ejection fraction (EF), to either receive vehicle or 60 mg/kg drug, daily, via gavage, for 4 weeks. No drug related mortality nor any changes in the body weight were observed (FIG. 75) during this time. Importantly, echocardiography data at 8 w post-MI (4 w post-treatment) showed that the cardiac dysfunction in vehicle-treated HF mice progressed during this time with increased end-systolic and end-diastolic volumes (ESV and EDV), and reduced ejection fraction (FIG. 76 and FIG. 77). In contrast, the cardiac function of compound 1 treated mice did not worsen, and the ESV, EDV, and EF did not change over 4 weeks of drug treatment. The cardiac function in drug treated mice was significantly better than the vehicle treated mice at 8 w post-MI. These differences in cardiac function were not due to heart rate, as the mean heart rate was more than 480 BPM at both time-points and was comparable between both groups (FIG. 78).

Compound 1 Treatment Ameliorates Cardiac Hypertrophy

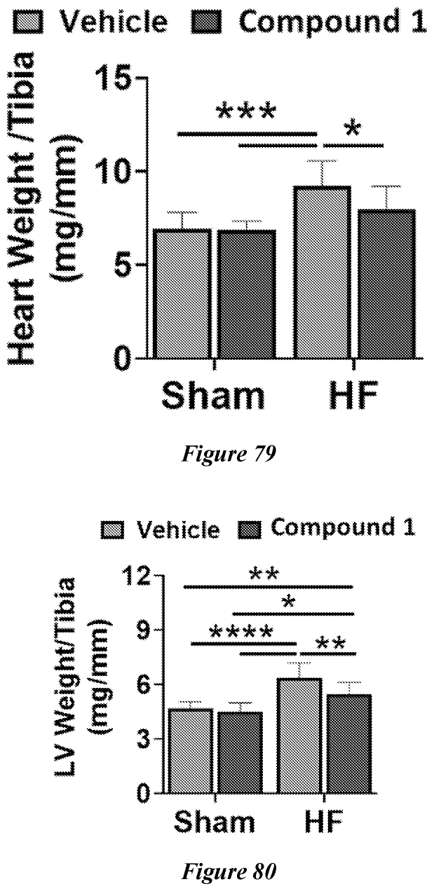

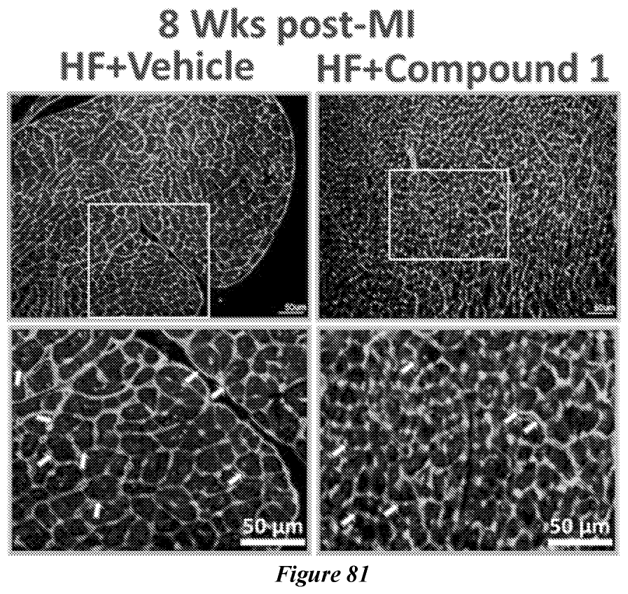

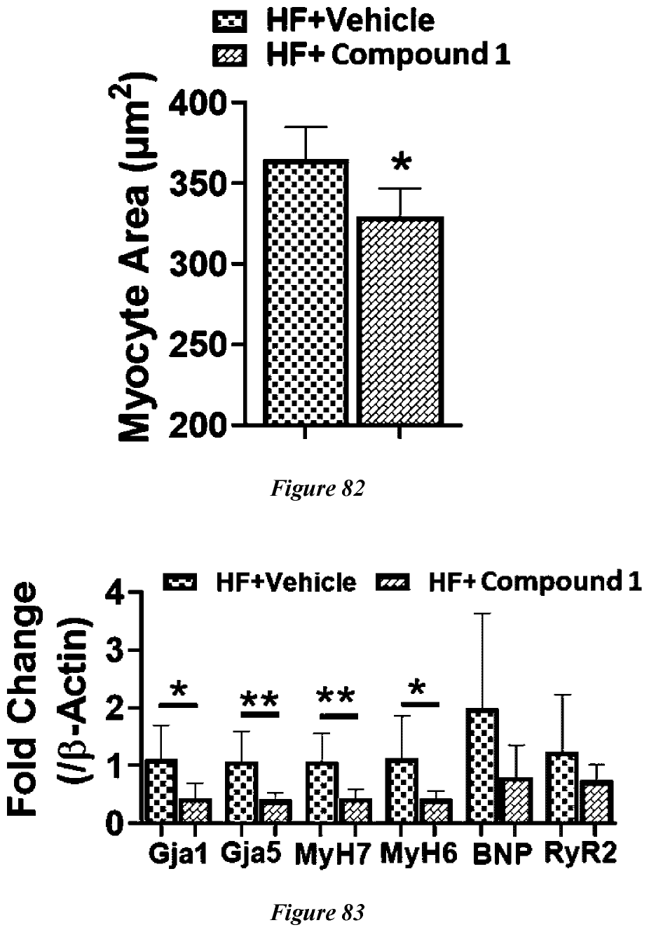

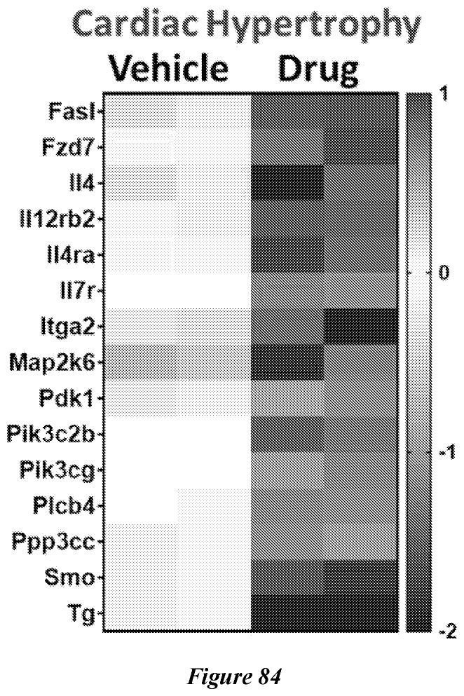

Gravimetric analysis of heart and LV weights showed that, while vehicle treated HF mice exhibited a significant increase in tibia normalized heart and LV weights, drug treatment from 4 to 8 w inhibited cardiac hypertrophy as reflected by significantly reduced heart (FIG. 79) and LV (FIG. 80) weights when compared with the vehicle treated HF mice. To further validate these results, WGA staining was conducted and cardiomyocyte area in LVs was measured. A shown in FIG. 81 and FIG. 82, drug treatment significantly decreased cardiomyocyte area and hypertrophy when compared with the vehicle treated HF mice. A significant decrease in the gene expression of several hypertrophy markers (such as Gja1, Gja5, MyH7, and MyH6) was also observed, while some others (such as BNP and RyR2) showed a decreasing trend (FIG. 83 and FIG. 84). This was also supported by RNA seq data obtained from drug treated CD4⁺ T-cells, as upon retrospective analysis it was observed that several signaling pathways in T-cells that mediate cardiac hypertrophy were downregulated with compound 1 treatment.

Compound 1 Treatment Specifically Depletes CD4+ Helper T-Cells in HF Mice

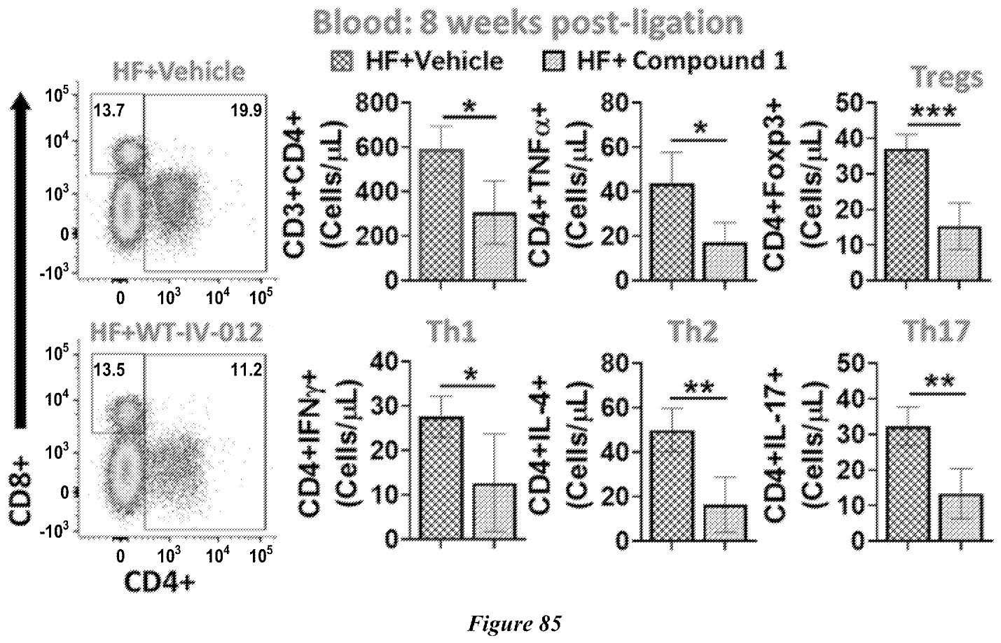

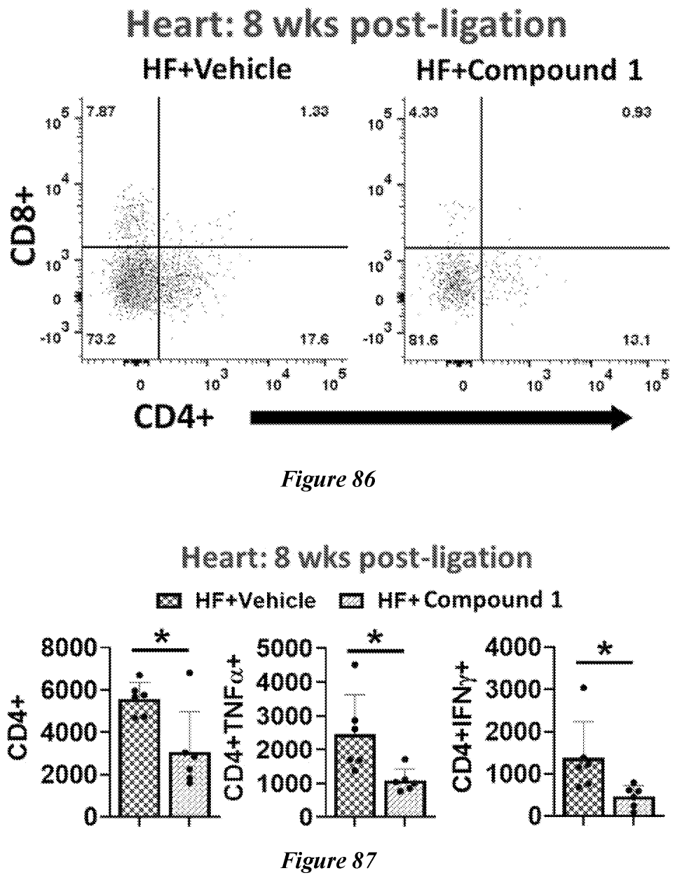

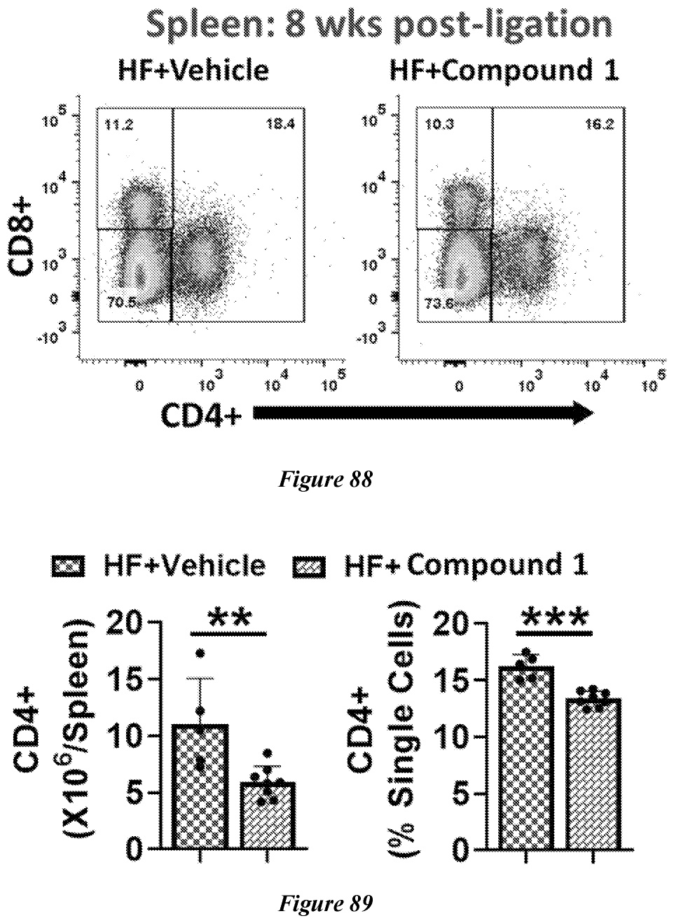

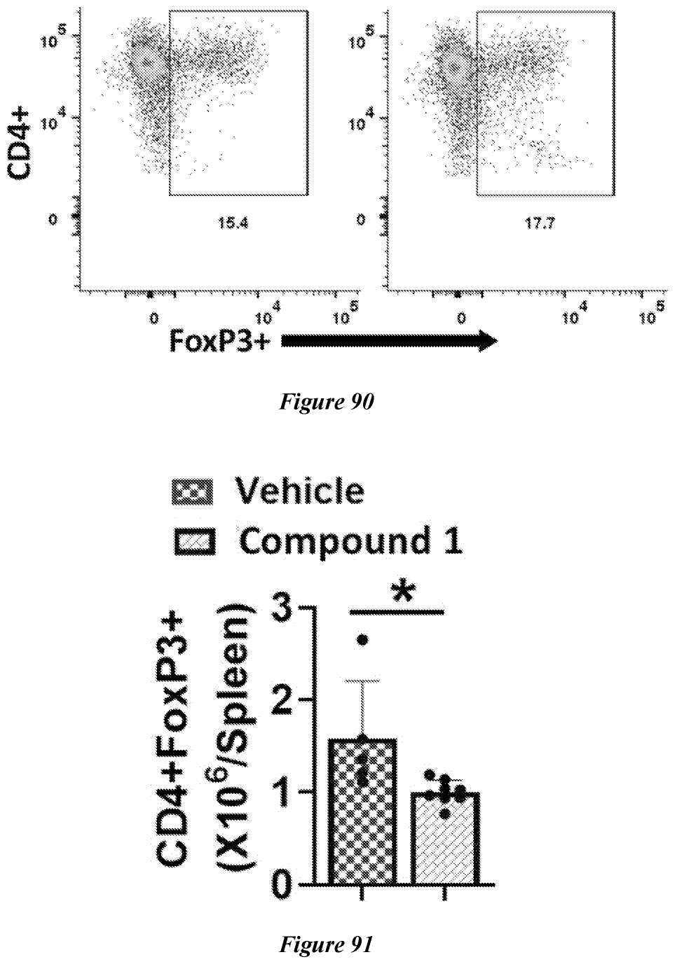

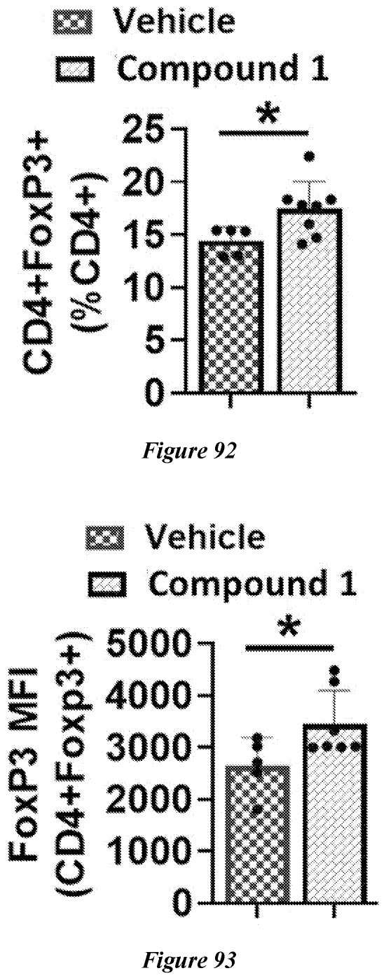

Using flow cytometry, CD4⁺ T-cells were measured in the circulation, failing hearts, and spleens of vehicle- and drug-treated mice at 8 w post-MI (4 w post-treatment). As shown in FIG. 85, daily treatments with the compound 1 significantly decreased circulating CD4⁺ T-cells at 8 w. Reduction in T-cell numbers was not limited to a particular helper T-cell subset, as numbers for both the pro- and anti-inflammatory cells, viz CD4⁺TNFα⁺, CD4⁺FoxP3⁺ Tregs, CD4⁺IFNγ⁺ Th1 T-cells, CD4⁺IL-4⁺ Th2 T-cells, and CD4⁺IL-17⁺ Th17 T-cells were decreased significantly. Similarly, a significant decrease in cardiac CD4⁺ T-cells in compound 1 treated HF mice was also observed when compared with the vehicle-treated mice (FIG. 86 and FIG. 87). This decrease was reflected in the significant decline of both TNF⁺ and IFNγ⁺ cells, suggesting inhibition of cytokine production in CD4⁺ T-cells, consistent with in-vitro T-cell inhibition assays. A similar decrease in CD4⁺ T-cell numbers (and frequency) was also observed in splenic CD4⁺ T-cells (FIG. 88 and FIG. 89), including diminished counts of FoxP3⁺ Tregs (FIG. 90 and FIG. 91). Interestingly, the overall frequency of Foxp3+ Tregs was increased in CD4+ T-cells (FIG. 92), suggesting that the other Th subsets were decreased more than the FoxP3+ Tregs. Since Foxp3 expression has been found to directly correlate with the immune-suppressive capacity of Tregs, its expression was also measured in vehicle and drug-treated mice. As shown in FIG. 93, drug treatment significantly increased FoxP3 expression (reflected as MFI) in Tregs, suggesting that the FoxP3+ Tregs remaining after the drug treatment were significantly potent and immune-suppressive. This was an interesting finding, as it was previously shown that Tregs undergo a pro-inflammatory (and pathological) phenotypic switch with loss of their immune-suppressive potential during chronic HF. Increased FoxP3 expression with compound 1 treatment, thus, indicates improved competence of Tregs in suppressing pathological immune activation during chronic HF.

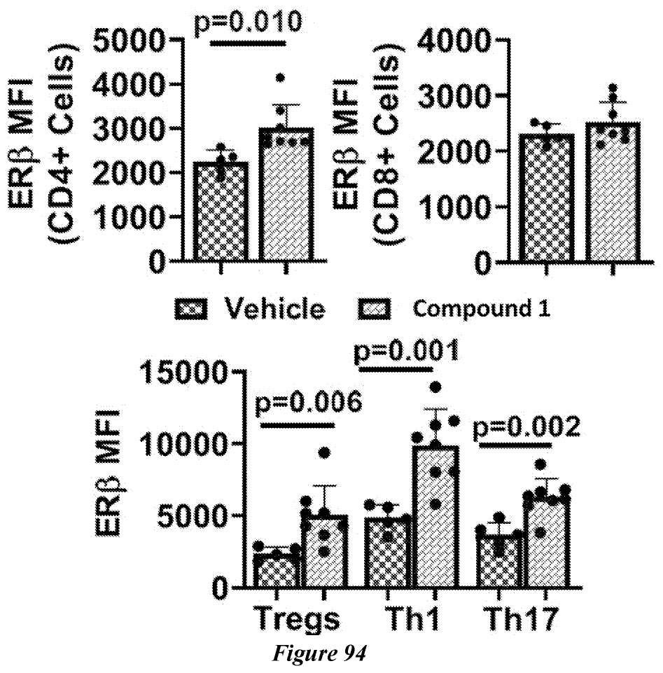

ERβ expression is stabilized/increased in splenic T-cells with compound 1 treatment (FIG. 94).

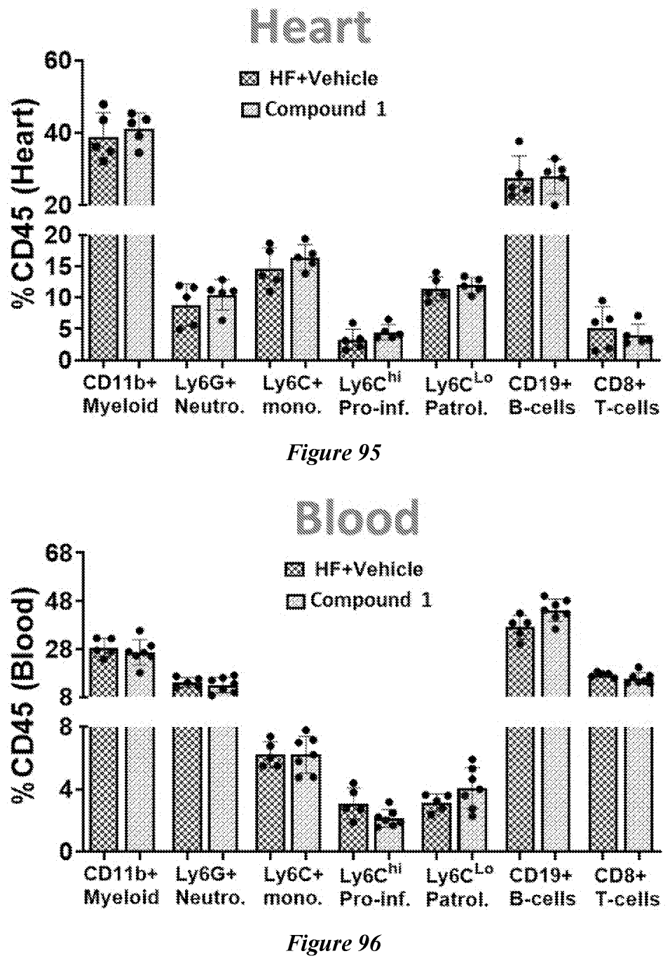

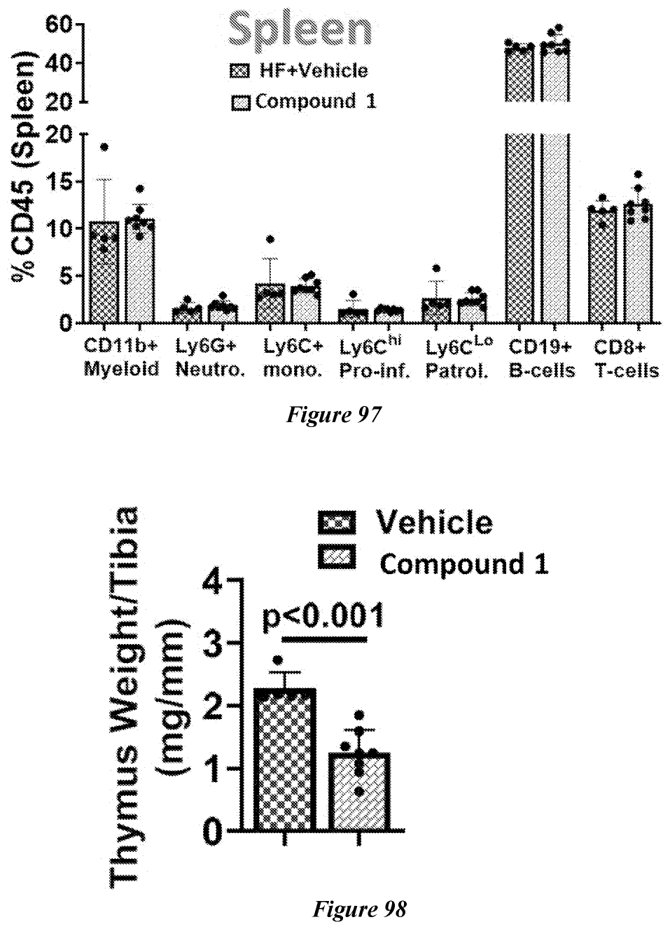

To determine the effects of compound 1 on other immune cells, also known to play a key role in LV remodeling during chronic HF, circulating, splenic, and cardiac levels of other immune cells of myeloid and lymphoid origin were measured. As shown in FIG. 95-FIG. 97, compound 1 did not alter the frequency of either the myeloid cells (CD11b⁺) such as Ly6G⁺ neutrophils or Ly6C⁺ monocytes/macrophages, or any other lymphocyte populations, such as CD19⁺ B-cells or CD8⁺ cytotoxic T-cells in the hearts (FIG. 95), blood (FIG. 96) or spleens (FIG. 97) of HF mice, suggesting highly specific effects of compound 1 on the CD4⁺ Helper T-cells.

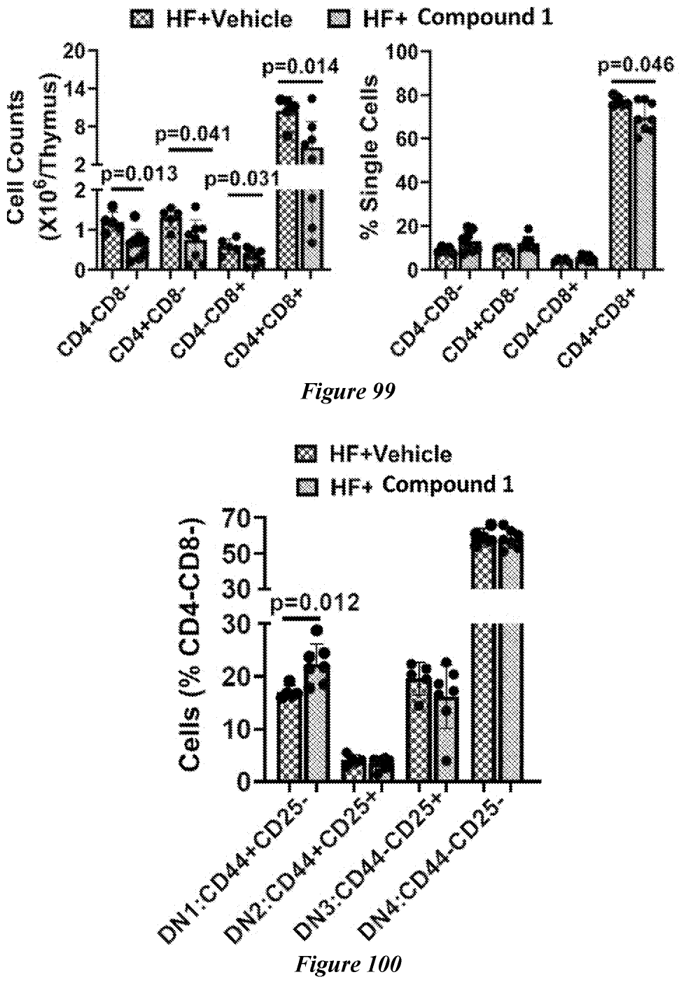

The effect of compound 1 on thymic cellularity and T-cell development was also investigated (FIG. 98-FIG. 100).

Discussion

Several findings were demonstrated in this study. First, ERα signaling is significantly upregulated in CD4⁺ T-cells during chronic HF. Second, while ERα expression is temporally regulated to significantly decrease during MI and increase during chronic HF, ERβ expression is spatially regulated and is increased specifically in T-cells infiltrated into the ischemic and failing hearts as compared to other cardiac immune cells. Third, ERβ agonists dose dependently inhibit CD4⁺ T-cell activation, proliferation, and the expression of pro-inflammatory cytokines both in males as well females to a similar extent. More importantly, this inhibition is specific to antigenic TCR-activation pathway. Fourth, ERβ agonists, if given early after MI, result in increased morbidity and mortality but improve cardiac function and reduce cellular hypertrophy if given during chronic HF. This underscores the critical and protective role of CD4⁺ T-cells in mediating wound-healing and scar formation post-MI but a pathological role during chronic HF, and points to significant immunological differences between the two, at least from the perspective of adaptive immunity. Fifth, ERβ agonists can selectively blunt CD4⁺ T-cell levels in the failing hearts, lymphoid tissues, and circulation without affecting other immune cells, suggesting that they can be developed as highly specific therapeutics for temporal modulation of pathological CD4⁺ T-cells without affecting other protective immune responses.

Canonical ER signaling is mediated either by binding of liganded ERs on EREs of target genes or indirectly by binding of (un)liganded ERs with other transcription factors such as AP-1 and NF-kB, to regulate activation, polarization, and proliferation of T-cells. ERα signaling is essential for: i) CD4+ T-cell activation against exogenous antigens, ii) polarization into IFNγ producing Th1 T-cells, and iii) trafficking and migration of activated CD4+ T-cells into tissues by regulating the expression of chemokine receptors such as CCR1, CCR2, CCR3, CCR4 and CCR5. In a limited cohort of human patients, it has been shown that systemic estradiol levels are increased during MI (first 3 days of hospitalization), are positively correlated with serum creatine phosphokinase levels, and very high estradiol levels are associated with increased mortality in the MI patients. Along the same lines, Th1/Th2 T-cell ratios have also been shown to increase in STEMI patients and are associated with increased adverse events. Together, these studies would suggest a pathological role of excessive estradiol mediated ERα signaling and Th1 T-cell polarization. However, preclinical studies have also shown that CD4+ T-cell activation during MI is protective and necessary for adequate healing and scar formation. The findings herein are consistent with these seemingly contrasting studies and underscore the importance of decreased ERα signaling in CD4⁺ T-cells at 3d post-MI to diminish non-antigenic T-cell activation and initiate regulated Th1 polarization. Nonetheless, these protective responses are considerably compromised during chronic HF. ERα levels and its downstream signaling is significantly upregulated to effect their antigen-dependent activation and pathological transitioning of CD4⁺ T-cells at 8 weeks post-MI, as has been previously shown. The data herein also shows that both during acute-MI and HF, CD4⁺ T-cells infiltrated into the ischemic and failing hearts had higher ERβ expression as compared to other immune cells of myeloid and lymphoid origin. This is highly intriguing considering the fact that HIF1α activation is known to increase ERβ signaling which, in turn, attenuates transcriptional activity of HIF1α. ERβ is also known to enhance integrin al and p1 expression in tumor cells to augment vinculin mediated adhesive potential, cellular motility and transmigration. Increased ERβ levels selectively in cardiac T-cells would, therefore, indicate its involvement either in i) regulating T-cell activation by antagonizing ERα, ii) amplifying integrin-mediated T-cell transmigration into the hearts or waning their exit, or iii) regulating HIF1α activation in T-cells to control ischemic damage.

Preclinical studies in pressure-overload and ischemic HF models have shown that T-cell activation during HF is antigen-dependent and is mediated by the TCR activation. It is, therefore, important that immuno-modulatory strategies targeting CD4+ T-cells specifically diminish TCR mediated T-cell activation to avoid general immune-suppression and infections by opportunistic pathogens. ER pathway is, thus, of significant therapeutic potential as ERα gene depletion from CD4+ T-cells result in defective TCR mediated T-cell activation by decreasing NFAT1, Zap70, and STAT5 levels. This defective TCR signaling could either be a direct consequence of lost ERα signaling or it could also be due to amplified ERβ signaling in the absence of ERα. The studies herein show latter to be the case, as the ERβ agonist dose-dependently inhibited the proliferation of TCR activated CD4⁺ T-cells in a gender-independent manner. ERα also regulates the gene expression of several T-cell specific cytokines (such as IFNγ, TNFα, IL-4, and IL-17), chemokine receptors (such as CCR1, CCR2, CCR3, CCR4 and CCR5), and other transcription factors (such as NF-kB and NFAT), all of which have been implicated in auto-immune diseases, including HF. Although, in the RNA sequencing data, no significant changes were observed in the expression of chemokines, compound 1 effectively inhibited expression of pro-inflammatory cytokines induced by TCR activation but failed to do so when PMA/Ionomycin was used as the stimulus. These findings suggest that ERs are not merely the accessory pathways to regulate the transcription and expression of these pro-inflammatory cytokines/activation markers, but are direct downstream regulators of TCR specific signaling.

ER signaling is a key pathway in promoting CD4+ T cell activation in autoimmune diseases such as MS, EAE, and Asthma. While high estradiol concentrations and ERα mediated Th1 polarization exacerbates MS; Th2 T cells promote EAE, suggesting that excessive polarization of T cells either to pro- or anti-inflammatory subsets disrupts the steady-state and can be pathological. It has also been shown that hearts form ERα^{-/-} mice are more prone to ventricular fibrillations and tachycardia, and exhibit increased cell death and reduced contractility. Alternatively, ERα agonists, but not ERβ, if given before I/R reduce infarct-size in female rabbits. This, in conjunction with the fact that whole body ERβ^{-/-} female mice exhibit increased mortality post-MI, suggest that ERα activation at the time of ischemic injury is cardio-protective and loss of ERβ is disrupts cardiac healing. Although, the cellular responders of protective ERα and ERβ signaling were not identified in these studies, others have shown that cardiac ERα or ERβ expression is not required for estradiol mediated cardio-protection. Along the same lines, it was previously shown that chronic HF is associated with a 2^{nd} wave of CD4⁺ T-cell activation, reflected as global increase in both pro- and anti-inflammatory helper T-cell subsets, in the circulation, spleen, mediastinal lymph nodes, and the failing hearts. Moreover, these T-cells exhibit a pathological phenotype and promote LV remodeling and progressive cardiac dysfunction as their depletion specifically from 4 to 8 weeks post-MI blunts progressive increases in ESV and EDV, and improves cardiac function. In contrast, studies by others have shown that CD4^{-/-} mice exhibit accentuated chamber dilatation and LV remodeling post-MI, suggesting their protective roles in mediating wound-healing, neovascularization, and fibrotic scar formation. These contradictory findings suggest that the CD4⁺ T-cells activated immediately after ischemic injury, as in MI, are immunologically and phenotypically different than those activated during chronic HF. However, the signaling mechanisms that mediate this transition from being protective during MI to pathological during HF are unknown. In these studies, it was observed that compound 1 mediated ERβ activation and CD4+ T-cell depletion at 7d post-MI resulted in significantly increased mortality but blunted LV remodeling and improved cardiac function upon administration from 4 to 8 weeks post-MI. These studies, for the first time, show that ER signaling could be one of important pathological phenotypic switch in CD4+ T-cells and selective ERβ agonists could provide a therapeutic immunomodulatory tool to inhibit ERα and blunt CD4⁺ T-cell activation and proliferation during HF. These results suggest that a balance of ERα and ERβ signaling in immune cells is critical during ischemic injury, further underscoring the key role of immune activation early after MI, and importance of identifying time-dependent changes in immune cells to determine optimal therapeutic window for temporal immune-modulation.

Expression of ERs is not restricted only to CD4+ T-cells and other immune cells, such as macrophages, neutrophils, dendritic cells, B-cells, and CD8+ T-cells, also express significant levels of these transcription factors. In monocytes and macrophages, estradiol exerts receptor dependent effects. While it promotes phagocytosis and pro-resolution via ERβ, it induces iNOS and, decreases Arg1 and IL-10 expression via ERα. In neutrophils, estradiol delays cell apoptosis and promotes netosis. In B-cells, estradiol mediated ERα signaling magnifies activation and inhibits apoptosis. Despite this widespread expression, compound 1 selectively inhibited CD4+ T-cells and did not affect other immune cells either in the circulation, spleen, or failing hearts. This specificity could be due to several factors. First, it could be due to differences in steady-state vs injury mediated activation of ERβ signaling. During steady-state, ERβ levels in CD4+ T-cells are significantly lower than the myeloid cells. However, during ischemic injury, significant amplification of ERβ expression selectively in cardiac CD4+ T-cells was observed, which was even higher than the myeloid cells, suggesting an important role of this pathway in mediating T-cells dependent protective responses. Second, differences in disease etiologies may activate disparate pathways in different immune cells. While myeloid cells activate ER signaling during trauma/hemorrhage; CD4+ T-cells probably activate this pathway specifically during autoimmune (ischemic/non-ischemic) injury. Specificity of the ERβ agonist only against the TCR mediated T-cell activation further supports this. Third, while ER signaling alters both activation/function and proliferation of CD4+ T-cells; it only regulates functional competency of other immune cells such as phagocytosis in monocyte/macrophages and netosis in neutrophils without altering their cell numbers.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating or preventing chronic heart failure in a subject following myocardial infarction, the method comprising:
administering to the subject an estrogen receptor β (ERβ) agonist during a maladaptive remodeling phase following the myocardial infarction.

2. The method of claim 1, wherein the ERβ agonist is not administered to the subject during a healing phase or a repair phase preceding the maladaptive remodeling phase.

3. The method of claim 1, wherein administration of the ERβ agonist commences at least 10 days following the myocardial infarction.

4. The method of claim 1, wherein the method further comprises assessing the subject to determine whether the subject has entered the maladaptive remodeling phase.

5. The method of claim 4, wherein assessing the subject to determine whether the subject has entered the maladaptive remodeling phase comprises:
measuring circulating CD4+ T-cell levels in the subject to determine when the subject has entered the maladaptive remodeling phase;
detecting one or more biomarkers in the subject to determine when the subject has entered the maladaptive remodeling phase: or
echocardiography, ventriculography, nuclear magnetic resonance, or any combination thereof.

6. The method of claim 4, wherein assessing the subject to determine whether the subject has entered the maladaptive remodeling phase comprises detecting one or more biomarkers in the subject to determine when the subject has entered the maladaptive remodeling phase, and wherein the one or more biomarkers are chosen from relative levels of myosin heavy chain isoforms, GLUT-1 expression level, alpha-actin expression level, natriuretic peptide expression level, galectin expression level, caveolin expression level, neuronal nitric oxide synthase expression level, angiotensin-converting enzyme expression level, GLUT-4 expression level, SERCA2a expression level, and a shift from glucose to fatty acid oxidation.

7. (canceled)

8. (canceled)

9. The method of claim 1, wherein the ERβ agonist is administered in an effective amount to:
inhibit activation and proliferation of CD4+ T-cells in the subject;
reduce circulating CD4+ T-cell levels in the subject;
or a combination thereof.

10. (canceled)

11. The method of claim 1, wherein the ERβ agonist is administered in an effective amount to reduce circulating CD4+ T-cell levels in the subject without significantly affecting circulating levels of neutrophils, monocytes, or B-cells.

12. The method of claim 1, wherein the ERβ agonist is administered in an effective amount to:
decrease left ventricular (LV) remodeling in the subject;
inhibit an increase in left ventricular end-diastolic volume in the subject;
inhibit an increase in left ventricular end-systolic volume in the subject;
or a combination thereof.

13. (canceled)

14. (canceled)

15. A method for inhibiting the activation and proliferation of CD4+ T-cells in the subject, the method comprising administering to the subject an estrogen receptor β (ERβ) agonist in an effective amount to inhibit activation and proliferation of CD4+ T-cells in the subject.

16. The method of claim 15, wherein the ERβ agonist is administered in an effective amount to reduce circulating CD4+ T-cell levels in the subject.

17. The method of claim 16, wherein the ERβ agonist is administered in an effective amount to reduce circulating CD4+ T-cell levels in the subject without significantly affecting circulating levels of neutrophils, monocytes, or B-cells.

18. A method of treating or preventing graft-versus-host disease, multiple sclerosis (MS), and/or experimental autoimmune encephalomyelitis (EAE) in a subject, the method comprising administering to the subject an estrogen receptor β (ERβ) agonist.

19. The method of claim 18, wherein the ERβ agonist is administered in an effective amount to:
inhibit activation and proliferation of CD4+ T-cells in the subject;
reduce circulating CD4+ T-cell levels in the subject;
or a combination thereof.

20. (canceled)

21. The method of claim 18, wherein the ERβ agonist is administered in an effective amount to reduce circulating CD4+ T-cell levels in the subject without significantly affecting circulating levels of neutrophils, monocytes, or B-cells.

22. The method of claim 1, wherein the ERβ agonist comprises a compound defined by any of Formula I-Formula XXXIII.

23. The method of claim 1, wherein the ERβ agonist comprises one of the following:

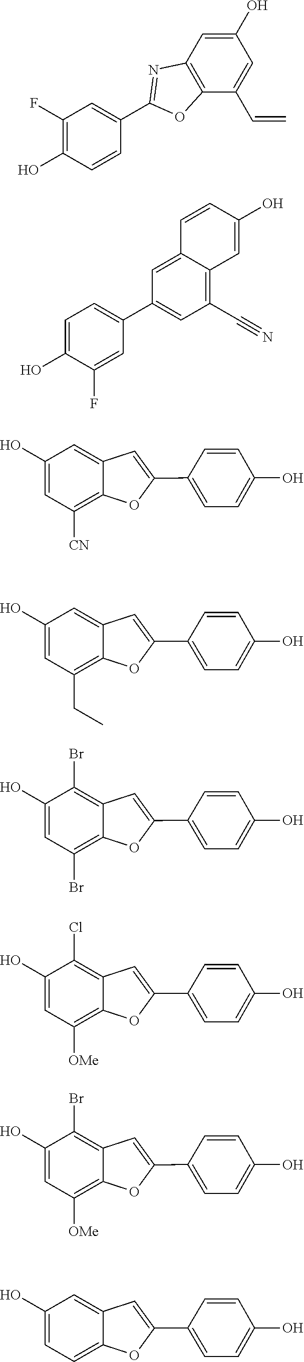

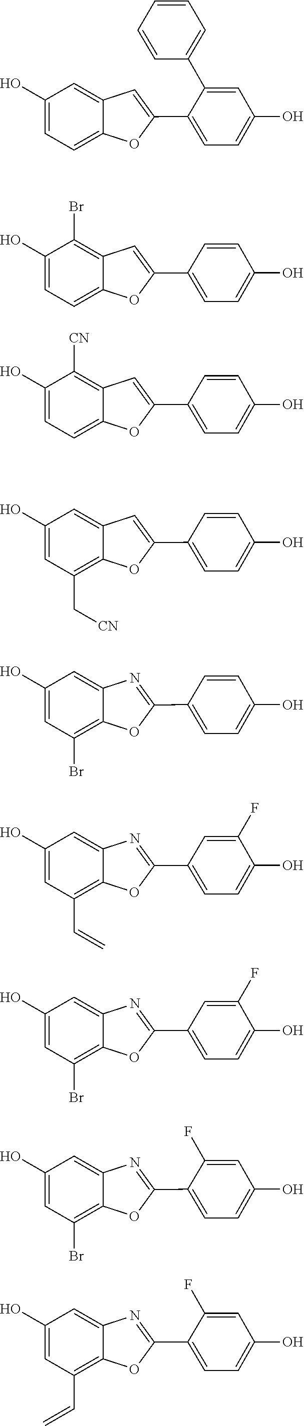

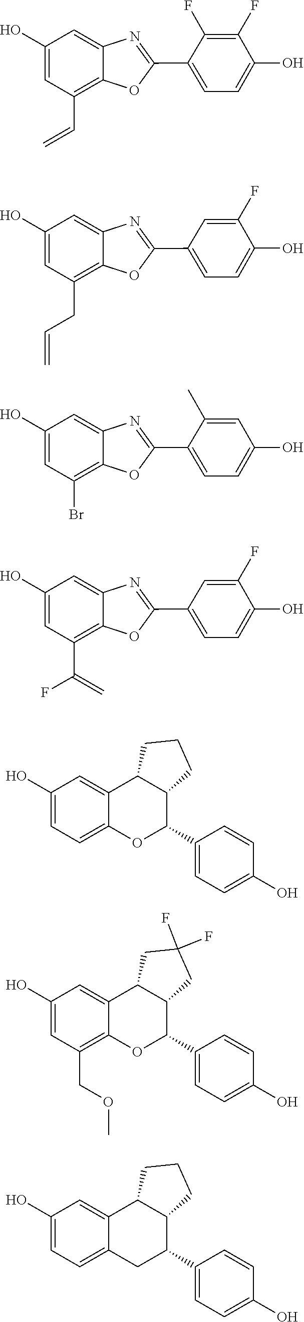

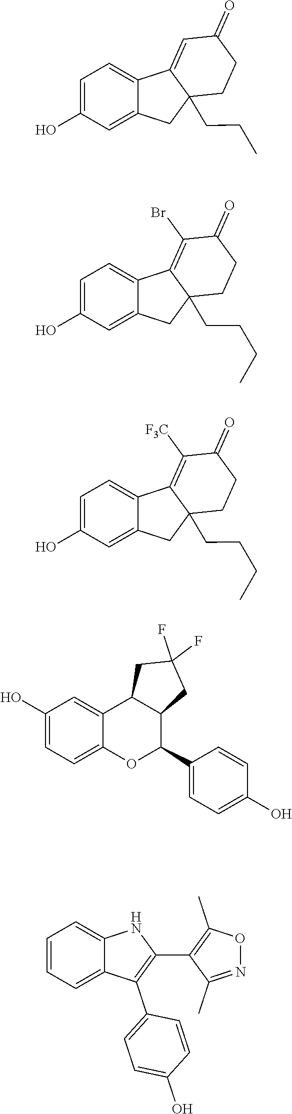

24. The method of claim 1, wherein the ERβ agonist has an EC₅₀ of 800 nM or less, wherein the ERβ agonist exhibits an ERβ-to-ERα agonist ratio of from 8 to 3000, or a combination thereof.

25. (canceled)

26. The method of claim 15, wherein the ERβ agonist comprises a compound defined by any of Formula I-Formula XXXIII and/or one of the following:

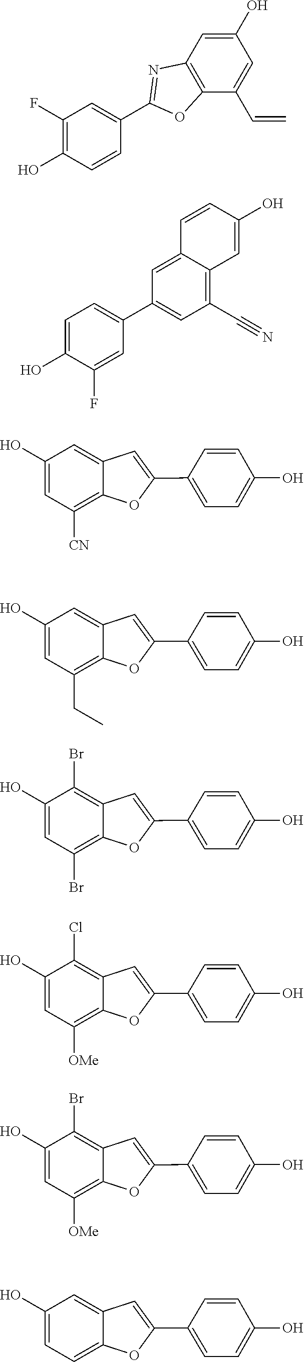

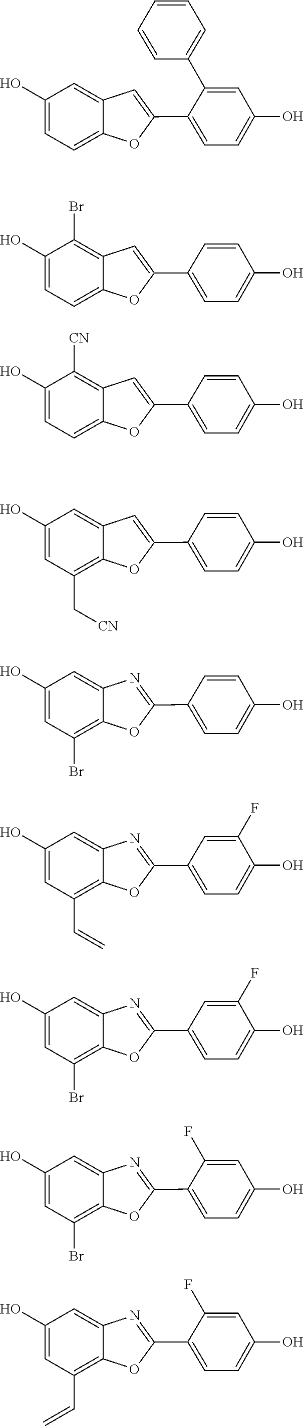

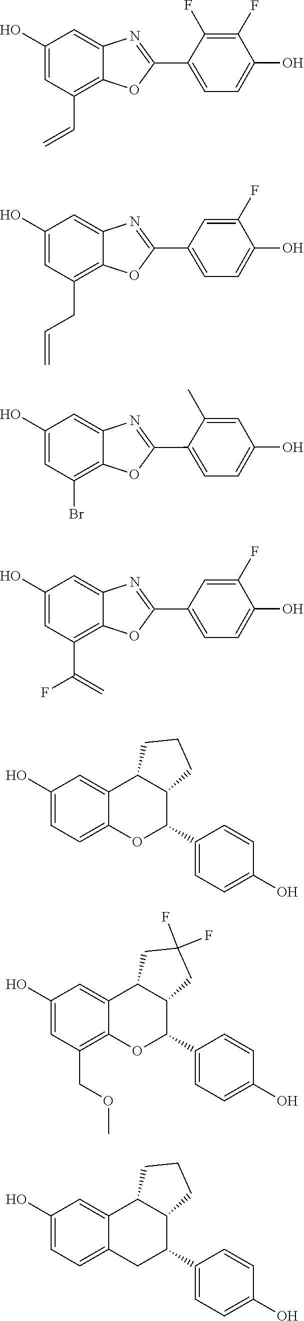

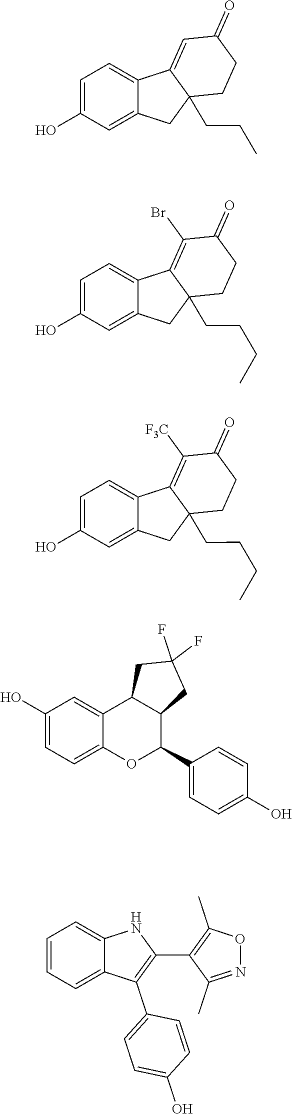

27. The method of claim 18, wherein the ERβ agonist comprises a compound defined by any of Formula I-Formula XXXIII and/or one of the following: 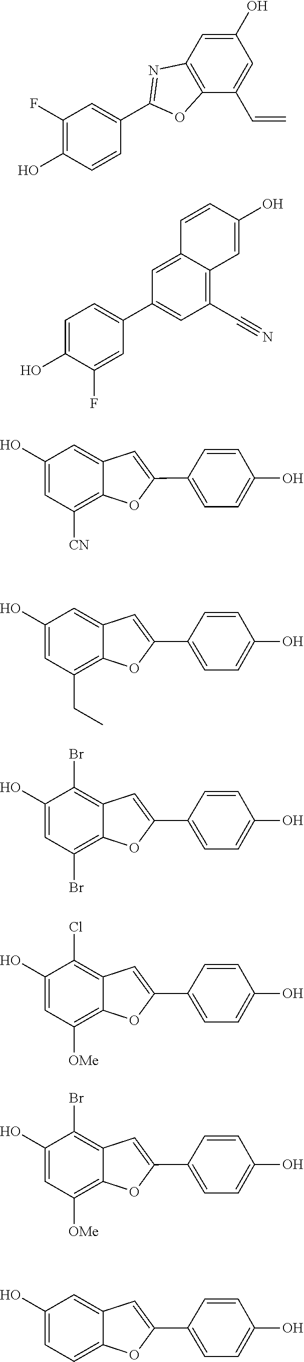 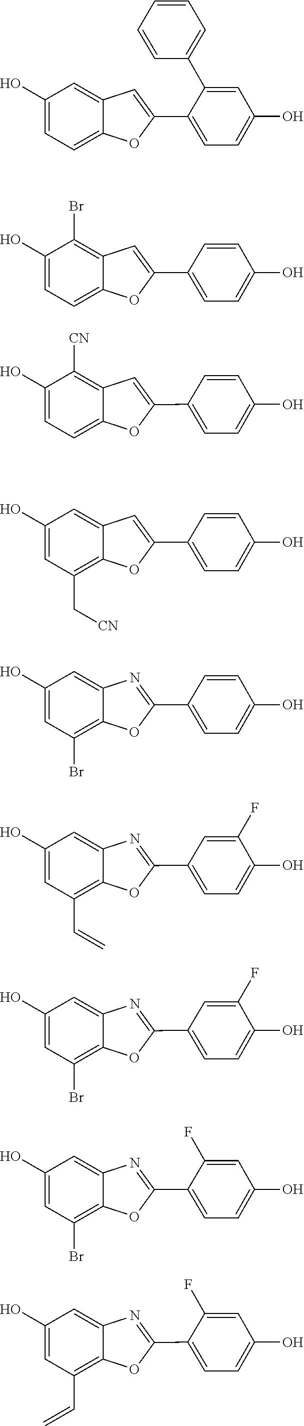 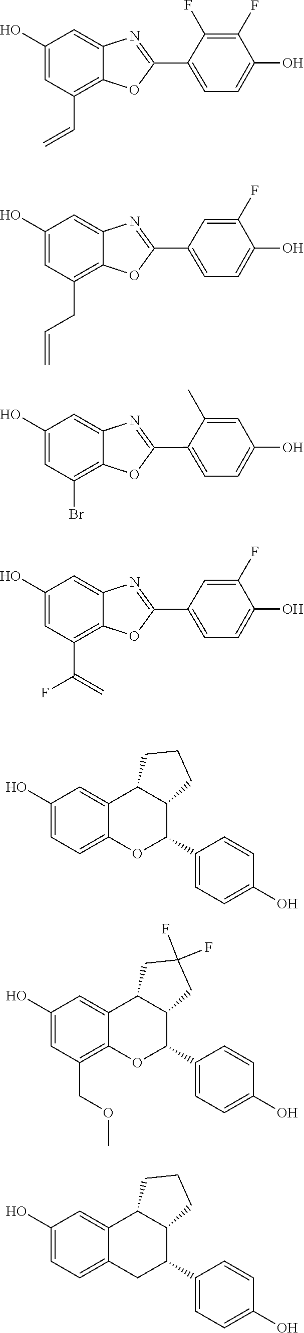 [chemical structure]
